(19) United States
(12) Patent Application Publication
(10) Pub. No.: US 2025/0090162 A1
(43) Pub. Date: Mar. 20, 2025

(54) VARIABLE TISSUE CONTRACTION

(71) Applicant: Edwards Lifesciences Innovation (Israel) Ltd., Caesarea (IL)

(72) Inventors: Mauricio Guerrero, Hawthorne, CA (US); Meena Francis, Trabuco Canyon, CA (US); Brian Patrick Murphy, Irvine, CA (US); Kaamran Ahsan Syed, Irvine, CA (US); Murrad Mirza Kazalbash, Tustin, CA (US); Khalid Jamal, Irvine, CA (US); Itschak Biran, Irvine, CA (US); David M. Taylor, Lake Forest, CA (US); Ehud Aviv, Costa Mesa, CA (US); Nicholas Scott Steenwyk, Minneapolis, MN (US); Sambhav Jain, Furlong, PA (US); Philip Jiun-An Chen, Mission Viejo, CA (US); Zhicheng Fang, Irvine, CA (US); Itay Avinathan, Tel-Aviv (IL)

(21) Appl. No.: 18/959,300

(22) Filed: Nov. 25, 2024

Related U.S. Application Data
(63) Continuation of application No. PCT/IB2023/055323, filed on May 24, 2023.
(60) Provisional application No. 63/370,609, filed on Aug. 5, 2022.
(60) Provisional application No. 63/369,571, filed on Jul. 27, 2022.
(60) Provisional application No. 63/346,387, filed on May 27, 2022.

(51) Int. Cl. A61B 17/04; A61B 17/00

(52) U.S. Cl. CPC A61B 17/0401; A61B 2017/00243; A61B 2017/0496

(57) ABSTRACT

An implant includes a tether and a series of anchors. Each anchor includes (i) a tissue-engaging element, (ii) a head that is slidably coupled to the tether and that includes a lock that is biased to lock in a manner that locks the head to the tether, and (iii) a push-button operatively coupled to the lock. A delivery tool includes a flexible tube that is transluminally advanceable to the heart, and a driver. The driver is configured to, for each of the anchors sequentially, (i) advance the anchor through the flexible tube to the heart while the lock is maintained unlocked by the flexible tube constraining the push-button, (ii) anchor the anchor to tissue of the heart, and (iii) release the push-button by deploying the head out of the flexible tube, the lock responsively locking. Other embodiments are also described.

### CROSS-REFERENCES TO RELATED APPLICATIONS

The present application is a Continuation of International Patent Application PCT/IB2023/055323 to Guerrero et al., filed May 24, 2023, which published as WO 2023/228098; which claims priority to:
Provisional U.S. Patent Application 63/346,387 to Biran et al., filed May 27, 2022;
Provisional U.S. Patent Application 63/369,571 to Biran et al., filed Jul. 27, 2022; and
Provisional U.S. Patent Application 63/370,609 to Biran et al., filed Aug. 5, 2022.

Each of the above documents is incorporated herein by reference in its entirety for all purposes.

### BACKGROUND

Annuloplasty involves remodeling tissue of an annulus. This can be done by pulling tissue about the annulus to a new shape. Tissue anchors can be used to facilitate medical procedures including annuloplasty, other remodeling of tissues, and securing implants. In some instances, tissue anchors can be used as an alternative to sutures. For example, a tissue anchor may be used for a procedure in which there is no line-of-sight to the target.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Described herein are systems, apparatus, and techniques for contracting tissue variably, e.g., such that different portions of tissue can be contracted to different degrees.

In some implementations, an implant can be implanted along a path along the tissue, and can be manipulated to contract one portion of the path more than another portion of the path. For example, an implant can have (or may be implanted to have) multiple sections (which can be elongate sections disposed colinearly and/or serially along a length of the implant) that are secured to the tissue, and whose length is adjustable independently of that of the other sections.

In some implementations, the length of each section can be lockable after its adjustment, e.g., before adjustment of another section.

In some implementations, each section of the implant is adjustable (and typically lockable) prior to implantation of a successive section of the implant.

In some implementations, a system can be provided, obtained, and/or used that includes such an implant, and a delivery tool for the implant. The delivery tool can be configured to deliver, secure, and/or adjust the implant.

In some implementations, the implant comprises a series of anchors, slidably coupled to a tether. In some implementations, each of the anchors has a head that is threaded onto the tether, and that is lockable to the tether.

In some implementations, the length of each section of the implant can be reduced by tensioning the tether, thereby drawing at least two of the anchors associated with that section (e.g., two anchors that delimit that section) closer to each other.

In some implementations, the locking of the head to the tether can lock the length of the section of the implant associated with (e.g., delimited by) that anchor.

In some implementations, the implant is a segmented implant that comprises a series of telescopic segments. In some implementations, the length of each section of the implant can be reduced by telescopically sliding one section into an adjacent section.

In some implementations, tension-modifiers are applied to an implant subsequently implantation of the implant.

In accordance with some implementations, a system and/or an apparatus (e.g., useable or for use at a heart of a subject) includes an implant (which can be the same as or similar to the implant above or other implants herein) that includes a tether and a series of anchors. In some implementations, each of the anchors can include a tissue-engaging element (e.g., one or more of a helix, screw, point, hook, barb, dart, arm, staple, sharpened portion, clip, pledget, etc.) and can optionally also include a head.

In some implementations, the anchor can be slidably coupled to the tether. In some implementations, the head can be coupled to the tissue-engaging element and/or slidably coupled to the tether. In some implementations, the head can include a lock configured, upon locking thereof, to lock the head to the tether.

In some implementations, the system/apparatus further includes a tensioning tool that includes a grasper, and that is configured to adjust tension on the tether by, for any anchor of the series, advancing to the anchor, grasping the tether at the anchor, and forming a loop from the tether by pulling the tether through the lock, the lock configured, upon locking thereof, to lock the head to the loop.

In some implementations, the grasper is configured to be advanced through the lock to grasp the tether.

In some implementations, the tensioning tool includes a flexible tube, the grasper configured to extend through the tube, the grasper is configured to pull the loop into the tube, and/or the tensioning tool is configured to release the bight once the lock has locked the head to the loop.

In some implementations, for each of the anchors: (i) the tube is configured to unlock the lock by applying an unlocking force to the lock, and/or (ii) the lock is biased to lock in an absence of the unlocking force.

In some implementations: (i) for each of the anchors, the lock includes a first locking arm and a second locking arm, each of the locking arms being hingedly coupled to the head, and/or (ii) the tube is configured to transition the lock toward the unlocked state by pushing against the locking arms in a manner that causes each of the locking arms to pivot away from each other.

In some implementations, the lock is configured to lock onto the bight upon withdrawal of the tube from the head such that the locking arms pivot towards each other.

In some implementations, the implant further includes a leading anchor, fixed to a distal end of the tether.

In some implementations, for each of the anchors, the tissue-engaging element is helical, defines a central longitudinal axis of the anchor, and is configured to be screwed into tissue of the heart by rotation of the tissue-engaging element around the central longitudinal axis.

In some implementations, for each of the anchors, the head is configured to facilitate screwing of the tissue-engaging element into the tissue while remaining slidably coupled to the tether.

In some implementations, for each of the anchors: (i) the head includes a circumferential wall that circumscribes the central longitudinal axis, and/or (ii) the head is slidably coupled to the tether by the tether extending transversely through the head via apertures defined in the circumferential wall.

In some implementations, for each of the anchors, the tissue-engaging element is rotatable independently of the circumferential wall.

In some implementations, for each of the anchors, the tissue-engaging element is rotatably locked with respect to the circumferential wall.

In some implementations, for each of the anchors, the lock is disposed medially from the circumferential wall.

In some implementations, for each of the anchors, the lock defines a press surface, and is configured to lock the head to the tether by pressing the press surface against the tether.

In some implementations, for each of the anchors, the lock includes a press plate that defines the press surface.

In some implementations, for each of the anchors, the lock includes a spring configured to lock the head to the tether by pressing the press surface against the tether.

In some implementations, for each of the anchors, the spring is configured to press the press surface against the tether by moving the press surface axially with respect to the tissue-engaging element.

In some implementations, for each of the anchors: (A) the head includes a casing and a lateral push-button, and/or (B) the lock is configured such that: (i) the spring moving the press surface axially with respect to the tissue-engaging element projects the push-button laterally from the casing, and/or (ii) pressing the push-button medially unlocks the lock by moving the press surface axially with respect to the casing.

In some implementations, for each of the anchors, the press surface is defined by a piston disposed within the casing, the piston further defines a first bearing surface, and the push-button defines a second bearing surface, oblique with respect to the first bearing surface, such that pressing the push-button medially moves the press surface axially with respect to the casing by sliding the second bearing surface over the first bearing surface.

In some implementations, for each of the anchors, the first bearing surface is oblique with respect to a central longitudinal axis of the anchor.

In some implementations, for each of the anchors, the second bearing surface is oblique with respect to a central longitudinal axis of the anchor.

In some implementations, for each of the anchors, the button is attached to a wedge that defines the second bearing surface.

In some implementations, for each of the anchors, the spring is configured to press the press surface against the tether by moving the press surface axially away from the tissue-engaging element.

In some implementations, for each of the anchors: (i) the head defines a circumferential wall that circumscribes the central longitudinal axis, (ii) the head is slidably coupled to the tether by the tether extending transversely through the head via an aperture defined in the circumferential wall, (iii) at least the press surface of the lock is disposed medially from the circumferential wall, and/or (iv) the spring is configured to press the press surface against the tether by moving the press surface across the aperture.

In some implementations, for each of the anchors, the tissue-engaging element is rotatable independently of the circumferential wall.

In some implementations, for each of the anchors, the tissue-engaging element is rotatably locked with respect to the circumferential wall.

In some implementations, for each of the anchors, (i) the head includes a casing including a proximal part and a base part, axially separated at a mid-section at which the circumferential wall is disposed, and/or (ii) the head is coupled to the tissue-engaging element via the base part, and the proximal part, the base part, and the tissue-engaging element are rotationally and axially locked with respect to each other and rotatably coupled to the circumferential wall.

In some implementations, the lock and the circumferential wall axially traverse the mid-section.

In some implementations, the system/apparatus further includes a delivery tool, configured to transluminally advance the anchors to the heart, and including: (i) a flexible tube, transluminally advanceable to the heart, and/or (ii) a driver, dimensioned to extend through the flexible tube.

In some implementations, the delivery tool is configured to, for each of the anchors, sequentially: (i) anchor the anchor to the tissue, such that a section of the tether extends to the anchor from a preceding one of the anchors, (ii) subsequently adjust tension on the section of the tether, and/or (iii) subsequently lock the lock.

In some implementations, for each of the anchors: (i) the lock is biased to lock, (ii) the head includes a casing, and a lateral push-button operatively coupled to the lock such that while the lock is unlocked the push-button projects laterally from the casing, and/or (iii) the delivery tool is configured to maintain the lock unlocked by constraining the push-button medially.

In some implementations, for each of the anchors, the delivery tool is configured to maintain the lock unlocked by the tube constraining the push-button medially, and to lock the lock by deploying the head out of the tube.

In some implementations, the casing of each anchor is keyed with an anchor key, and the tube is complementarily keyed with a drive key such that the delivery tool can apply torque to the anchor via rotation of the tube.

In some implementations, for each anchor, the push-button is disposed at the anchor key.

In some implementations, for each anchor, the anchor key includes a sunken key, the push-button is disposed at the sunken key, and the delivery tool is configured to maintain the lock unlocked by the drive key constraining the push-button medially.

In some implementations, for each of the anchors, the lock is biased to lock, the delivery tool is configured to constrain the anchor unlocked while adjusting the tension on the section of the tether.

In some implementations, for each of the anchors, the delivery tool is configured to constrain the anchor unlocked while transluminally advancing the anchor to the heart.

In some implementations, for each of the anchors, the delivery tool is configured to constrain the anchor unlocked while anchoring the anchor to the tissue.

In some implementations, for each of the anchors, the delivery tool is configured to transluminally advance the anchor to the heart subsequently to anchoring the preceding one of the anchors to the tissue.

In some implementations, for each of the anchors, the delivery tool is configured to transluminally advance the anchor to the heart subsequently to locking the lock of the preceding one of the anchors.

In accordance with some implementations, a method (e.g., useable or for use at a tissue of a heart of a subject) includes percutaneously implanting an elongate implant along the tissue such that the implant defines a path along the tissue by: (i) anchoring a first section of the implant along a first portion of the path, and/or (ii) subsequently anchoring a second section of the implant along a second portion of the path.

In some implementations, the method includes subsequently to anchoring the first section and prior to anchoring the second section: (i) contracting the first portion of the path by reducing a length of the first section, and/or (ii) while the first portion of the path remains contracted, fixing the length of the first section.

In some implementations, the method further includes subsequently to anchoring the second section, and while the length of the first section remains fixed: (i) contracting the second portion of the path by reducing a length of the second section, and/or (ii) while the second portion of the path remains contracted, fixing the length of the second section.

In some implementations: (i) the implant includes a first segment, a second segment, and a third segment, telescopically coupled to each other in series, (ii) the first section of the implant includes part of the first segment and part of the second segment, (iii) the second section of the implant includes part of the second segment and part of the third segment, (iv) contracting the first portion of the path includes contracting the first portion of the path by reducing the length of the first section by telescopically contracting the first segment into the second segment, and/or (v) contracting the second portion of the path includes contracting the second portion of the path by reducing the length of the second section by telescopically contracting the second segment into the third segment.

In some implementations: (i) fixing the length of the first section includes locking the first segment to the second segment, and/or (ii) fixing the length of the second section includes locking the second segment to the third segment.

In some implementations: (i) the first segment includes a first anchor, the second segment includes a second anchor, and a third segment includes a third anchor, (ii) anchoring the first section of the implant includes anchoring the first anchor and the second anchor to the tissue, and/or (iii) anchoring the second section of the implant includes anchoring the second anchor and the third anchor to the tissue.

In some implementations: (i) the implant includes a tether, (ii) contracting the first portion of the path includes contracting the first portion of the path by reducing a length of the first section by applying tension to the tether, and/or (iii) contracting the second portion of the path includes contracting the second portion of the path by reducing a length of the second section by applying tension to the tether.

In some implementations, percutaneously implanting the implant along the tissue includes implanting the implant along the tissue such that the tether defines the path along the tissue.

In some implementations: (i) fixing the length of the first section includes locking a first lock to the tether, and/or (ii) fixing the length of the second section includes locking a second lock to the tether.

In some implementations, the method further includes percutaneously advancing the first lock to the heart subsequently to anchoring the first section of the implant along the first portion of the path.

In some implementations, advancing the first lock to the heart includes advancing the first lock to the heart prior to anchoring the second section of the implant along the second portion of the path.

In some implementations, percutaneously advancing the first lock to the heart includes percutaneously sliding the lock over and along the tether.

In some implementations, the implant includes a first locking anchor and a second locking anchor, each including: (i) a tissue-engaging element (e.g., one or more of a helix, screw, point, hook, barb, dart, arm, staple, sharpened portion, clip, pledget, etc.), and/or (ii) a head that is attached to the tissue-engaging element, is slidably coupled to the tether, and includes a lock.

In some implementations, percutaneously implanting the implant along the tissue includes anchoring the first and second locking anchors to the tissue.

In some implementations, the first lock is the lock of the first locking anchor, and fixing the length of the first section includes locking the lock of the first locking anchor to the tether. In some implementations, the second lock is the lock of the second locking anchor, and fixing the length of the second section includes locking the lock of the second locking anchor to the tether.

In some implementations, the method further includes percutaneously sliding each of the first and second locking anchors over and along the tether to the heart.

In some implementations, percutaneously implanting the implant along the tissue includes securing a distal part of the tether to the tissue by anchoring to the tissue a leading anchor, coupled to the tether.

In some implementations, percutaneously sliding each of the first and second locking anchors over and along the tether to the heart includes, subsequently to securing the distal part of the tether to the tissue by anchoring the leading anchor to the tissue, percutaneously sliding each of the first and second locking anchors distally over and along the tether toward the leading anchor.

In some implementations, anchoring the first and second locking anchors to the tissue includes anchoring the first and second locking anchors to the tissue using an anchor driver.

In some implementations, for each of the first and second locking anchors, the lock is biased toward locking, and anchoring the locking anchor to the tissue includes anchoring the locking anchor to the tissue while the anchor driver constrains the lock unlocked.

In some implementations, for each of the first and second locking anchors, locking the lock to the tether includes releasing the lock such that the lock responsively locks.

In some implementations, for each of the first and second locking anchors, anchoring the locking anchor to the tissue while the anchor driver constrains the lock unlocked includes anchoring the locking anchor to the tissue while the anchor driver constrains the lock unlocked by inhibiting a push-button of the head from moving laterally outward from a casing of the head.

In some implementations, for each of the first and second locking anchors, locking the lock to the tether includes, using the anchor driver, applying a locking force to the lock.

In some implementations, for each of the first and second locking anchors, the locking force is a rotational locking force, and applying the locking force to the lock includes applying the rotational locking force to the lock.

The above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

In accordance with some implementations, a method (e.g., useable or for use at a tissue of a heart of a subject) includes: (i) percutaneously anchoring a first anchor of an implant to a first site of the tissue, and/or (ii) subsequently, percutaneously anchoring a second anchor of the implant to a second site of the tissue, a first portion of the tissue being disposed between the first site and the second site. In some implementations, the method includes, subsequently, contracting the first portion of the tissue by drawing together the first anchor and the second anchor. In some implementations, while the first portion of the tissue remains contracted, the method includes fixing a distance between the first anchor and the second anchor.

In some implementations, the method includes, subsequently, percutaneously changing a distance between a third anchor of the implant and the second anchor by sliding the third anchor with respect to the second anchor.

In some implementations, the method includes, subsequently, anchoring the third anchor to a third site of the tissue, a second portion of the tissue being disposed between the second site and the third site, and subsequently, while the distance between the first anchor and the second anchor remains fixed, contracting the second portion of the tissue by drawing together the second anchor and the third anchor.

In some implementations, the method also includes, while the second portion of the tissue remains contracted, fixing a distance between the second anchor and the third anchor.

In some implementations: the implant includes a tether, and percutaneously anchoring the second anchor includes percutaneously anchoring the second anchor while the tether is coupled to the first anchor and is slidably coupled to the second anchor.

In some implementations, drawing together the first anchor and the second anchor includes sliding the tether with respect to the second anchor.

In some implementations, fixing the distance between the first anchor and the second anchor includes inhibiting sliding of the tether with respect to the second anchor by locking a first lock to the tether.

In some implementations, drawing together the second anchor and the third anchor includes sliding the tether with respect to the third anchor.

In some implementations, fixing the distance between the second anchor and the third anchor includes locking a second lock to the tether.

In some implementations, sliding the tether with respect to the second anchor includes sliding the tether in a first direction with respect to the second anchor.

In some implementations, locking the first lock to the tether includes inhibiting sliding of the tether in a second direction with respect to the second anchor by locking the first lock to the tether, the second direction being opposite to the first direction.

In some implementations, the method further includes inhibiting further sliding of the tether in the first direction with respect to the second anchor by locking a third lock to the tether.

In some implementations, locking the third lock to the tether includes locking the third lock to the tether prior to drawing together the second anchor and the third anchor.

The above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

In accordance with some implementations, a method (e.g., usable at or for use at a tissue of a heart of a subject) includes percutaneously implanting a first section of an implant along a first portion of the tissue by anchoring a first anchor of the implant to a first site of the tissue, and anchoring a second anchor of the implant to a second site of the tissue, the first portion of the tissue being disposed between the first site and the second site.

In some implementations, the method also includes, subsequently, contracting the first portion of the tissue by reducing a length of the first section. In some implementations, the method includes, while the first portion of the tissue remains contracted, fixing the length of the first section.

In some implementations, the method includes, subsequently, percutaneously implanting a second section of the implant along a second portion of the tissue by anchoring a third anchor of the implant to a third site of the tissue.

In some implementations, the method includes, subsequently, while the length of the first section remains fixed, contracting the second portion of the tissue by reducing a length of the second section. In some implementations, the method includes, while the second portion of the tissue remains contracted, fixing the length of the second section.

In some implementations, the second portion of the tissue is disposed between the second site and the third site, and implanting the second section of the implant along the second portion of the tissue includes implanting the second section of the implant along the second portion of the tissue that is disposed between the second site and the third site.

In some implementations: the second portion of the tissue is disposed between the third site and a fourth site, and implanting the second section of the implant along the second portion of the tissue includes implanting the second section of the implant along the second portion of the tissue by anchoring the third anchor of the implant to the third site of the tissue, and anchoring a fourth anchor of the implant to the fourth site of the tissue.

In accordance with some implementations, a system and/or an apparatus (e.g., useable at or for use at a heart of a subject) includes: (i) a segmented implant that includes multiple segments, telescopically coupled to each other in series, each of the segments including an anchor; and/or (ii) a delivery tool.

In some implementations, the delivery tool is configured to: (i) percutaneously deliver the implant to the heart, (ii) anchor each of the anchors to tissue of the heart, and/or (iii) contract the tissue by telescopically contracting the implant while each of the anchors remain anchored to the tissue.

In some implementations, the delivery tool is configured to telescopically extend the implant within the heart.

In some implementations, each of the segments is articulatable with respect to an adjacent one of the segments.

In some implementations, each of the segments further includes a lock, configured such that, for each of the segments, locking of the lock fixes an amount of telescoping between the segment and an adjacent one of the segments.

In some implementations, each of the anchors is biased toward assuming an anchoring position, and the delivery tool is configured: (i) to percutaneously deliver the implant to the heart while each of the anchors is constrained in a delivery position, and/or (ii) within the heart, release each of the anchors to allow each of the anchors to responsively move toward the anchoring position.

In some implementations, the implant has a delivery state in which, for each of the segments, the anchor is constrained in the delivery position by an adjacent one of the segments.

In some implementations, in the delivery state, for each of the segments, the anchor is constrained in the delivery position by being disposed within an adjacent one of the segments.

In some implementations, for each of the segments, the delivery tool is configured to release the anchor by telescopically extending the segment out of the adjacent one of the segments.

In some implementations, the delivery tool is configured to secure a first section of the implant (e.g., an elongate member thereof) along a first portion of a path along the tissue by: (i) anchoring the anchor of a first segment of the multiple segments to a first site on the tissue, and/or (ii) anchoring the anchor of a second segment of the multiple segments to a second site on the tissue.

In some implementations, the delivery tool is configured to secure a second section of the implant (e.g., an elongate member thereof) along a second portion of a path along the tissue, by anchoring the anchor of a third segment of the multiple segments to a third site on the tissue, the first section including part of the first segment and part of the second segment, and the second section including part of the second segment and part of the third segment.

In some implementations, the delivery tool is configured to contract the tissue at the first portion of the path by telescopically contracting the first segment into the second segment.

In some implementations, the delivery tool is configured to contract the tissue at the second portion of the path by contracting the second segment into the third segment independently of contracting the first segment into the second segment.

In some implementations, the delivery tool is configured to lock the first segment to the second segment subsequently to telescopically contracting the first segment into the second segment, and to lock the second segment to the third segment subsequently to telescopically contracting the second segment into the third segment.

In some implementations, the delivery tool is configured to contract the tissue at the second portion of the path by contracting the second segment into the third segment subsequently to locking the first segment to the second segment.

In some implementations, the delivery tool is configured to telescopically extend the implant within the heart by: (i) telescopically extending the first segment from the second segment prior to anchoring the anchor of the second segment, and/or (ii) telescopically extending the second segment from the third segment subsequently to anchoring the anchor of the second segment and prior to anchoring the anchor of the third segment.

The above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

In accordance with some implementations, a system (e.g., useable at or for use at a tissue of a heart of a subject) includes an implant and a delivery tool. In some implementations, the implant includes an elongate member, multiple anchors, and multiple locks.

In some implementations, the delivery tool is configured to implant the implant at the tissue such that the elongate member defines a path along the tissue, by: (i) using one or more of the anchors to anchor a first section of the elongate member along a first portion of the path, and (ii) subsequently, using one or more of the anchors to anchor a second section of the elongate member along a second portion of the path.

In some implementations, the delivery tool is configured to contract the tissue by: (i) subsequently to anchoring the first section and prior to anchoring the second section, contracting the first portion of the path by reducing a length of the first section, (ii) while the first portion of the path remains contracted, using at least a first lock of the multiple locks to fix the length of the first section, (iii) subsequently to anchoring the second section, and while the length of the first section remains fixed, contracting the second portion of the path by reducing a length of the second section, and/or (iv) while the second portion of the path remains contracted, using at least a second lock of the multiple locks to fix the length of the second section.

In accordance with some implementations, a system and/or an apparatus (e.g., useable at or for use at a heart of a subject) includes an implant, multiple tension-modifiers, and a delivery tool. In some implementations, the implant includes: (i) a tether, and/or (ii) a series of anchors, each of the anchors including a tissue-engaging element (e.g., one or more of a helix, screw, point, hook, barb, dart, arm, staple, sharpened portion, clip, pledget, etc.) and, optionally, also including a head coupled to the tissue-engaging element. In some implementations, the anchor (e.g., a head thereof, etc.) is slidably coupled to the tether.

In some implementations, the delivery tool includes: (i) a tube, transluminally advanceable to the heart, (ii) an anchor driver, configured to advance the anchors through the tube and, for each of the anchors sequentially, intracardially anchor the anchor to tissue of the heart by driving the tissue-engaging element into the tissue, and/or (iii) an applicator, configured to intracardially connect the tension-modifiers to the implant.

In some implementations, the applicator is configured to intracardially connect the tension-modifiers to adjacent anchors of the series.

In some implementations, the applicator is configured to intracardially connect the tension-modifiers to the tether.

In some implementations, the applicator is configured to intracardially connect the tension-modifiers to the tether between adjacent anchors of the series.

In some implementations, each of the tension-modifiers is a connector, configured to connect a respective pair of adjacent anchors of the series.

In some implementations, each of the tension-modifiers is configured to draw the respective pair of anchors toward each other.

In some implementations, each of the tension-modifiers is configured to inhibit the respective pair of anchors from moving away from each other.

In some implementations, each of the tension-modifiers is a spacer, configured to be connected to the tether between a respective pair of anchors of the series.

In some implementations, each of the spacers is tubular.

In some implementations, for each of the spacers, the spacer applicator is configured to intracardially connect the spacer to the tether while the spacer is constrained in an open state in which the spacer has a substantially C-shaped cross section.

In some implementations, the tube defines a primary lumen, the anchor driver is configured to advance each of the anchors out of the lumen, and the spacer applicator is configured to advance each of the spacers out of a secondary lumen disposed laterally from the primary lumen.

In accordance with some implementations, a method includes: (i) implanting an implant along a tissue of a heart of a subject (e.g., a living subject, a simulation, etc.) by anchoring to the tissue a series of anchors of the implant, the anchors being slidably coupled to a tether of the implant; (ii) subsequently, applying tension to the tether; and/or (iii) subsequently, adjusting the tension by applying a tension-modifier between a pair of adjacent anchors of the series.

In some implementations, the tension-modifier includes a spacer, and applying the tension-modifier between the pair of adjacent anchors includes urging the pair of adjacent anchors away from each other by connecting the spacer to the tether between the pair of adjacent anchors.

In some implementations, the tension-modifier includes a connector, and applying the tension-modifier between the pair of adjacent anchors includes drawing the pair of adjacent anchors toward each other by connecting the connector to both anchors of the pair of adjacent anchors.

The above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

In accordance with some implementations, a method includes: (i) identifying a subject (e.g., a living subject, a simulation, etc.) in whom an implant has been implanted along a tissue of a heart of the subject, the implant including a series of anchors slidably coupled to a tether, and the implant having been implanted by anchoring the series of anchors to the tissue; and/or (ii) in response to the identifying, adjusting tension on the tether by applying a tension-modifier between a pair of adjacent anchors of the series.

In some implementations, the tension-modifier includes a spacer, and applying the tension-modifier between the pair of adjacent anchors includes urging the pair of adjacent anchors away from each other by connecting the spacer to the tether between the pair of adjacent anchors.

In some implementations, the tension-modifier includes a connector, and applying the tension-modifier between the pair of adjacent anchors includes drawing the pair of adjacent anchors toward each other by connecting the connector to both anchors of the pair of adjacent anchors.

In accordance with some implementations, a system and/or an apparatus (e.g., useable with or for use with a tether) includes a lock that includes: (i) a tubular wall circumscribing a lumen dimensioned to receive the tether therethrough; (ii) a window, cut out of the wall; and/or (iii) a tab, cut from the wall, and positioned opposite the window, and biased to deflect across the lumen and protrude into the window in a manner that locks the lock to the tether received through the lumen.

In some implementations, the window is a first window of a plurality of windows cut out of the wall.

In some implementations, the tab is a first tab of a plurality of tabs cut from the wall.

In some implementations, each tab of the plurality of tabs is biased to deflect across the lumen and protrude into a respective window that faces that tab, in a manner that locks the lock to the tether.

In some implementations, the system/apparatus further includes a cutter. In some implementations, the cutter includes a cutting element (e.g., blade, knife, scissors, pincer, razor, scalpel, edge, sharp edge, cutting edge, etc.) dimensioned to receive the tether therethrough.

In some implementations, a distal part of the cutting element defines a pair of cutting edges facing each other.

In some implementations, the cutter includes an overtube that is slidable over and along the cutting element in a manner in which the cutting edges move towards each other, thereby cutting the tether.

In some implementations, the cutting element is manufactured from a single piece of stock tubing.

In some implementations, the cutter is advanceable to the heart over and along the tether while maintaining the overtube stationary with respect to the cutting element.

In some implementations, the cutter is adapted to transluminally advance the lock over and along the tether to a heart of a subject (e.g., a living subject, a simulation, etc.).

In some implementations, the cutter is adapted to transluminally advance the lock over and along the tether to the heart by pushing a distal end of the cutter against a proximal end of the lock.

In some implementations, the cutting element includes a pair of arms, each arm having: (i) a proximal part that extends distally and away from the proximal part of the other arm of the pair, and/or (ii) a distal part that extends distally and towards the distal part of the other arm of the pair.

In some implementations, each arm defines a discrete elbow between the proximal part and the distal part.

In some implementations, each arm defines a continuous curve from the proximal part to the distal part.

In some implementations, for each arm of the pair, a cutting edge of the pair of cutting edges is defined by the distal part of the arm.

In some implementations, the proximal parts extend outwardly such that the arms collectively define a wider diameter than an inner diameter of the overtube, such that sliding the overtube distally over and along the proximal parts squeezes together the proximal parts, causing the arms to pivot inwardly towards each other.

In some implementations, for each arm of the pair, an angle at which the proximal part is disposed with respect to a central axis of the cutter is shallower than an angle at which the distal part is disposed with respect to the central axis.

In some implementations, for each arm of the pair, the proximal part is longer than the distal part.

In some implementations, the system/apparatus further includes an obstructing rod, adapted to maintain the lock in an unlocked state in which the tab is prevented from deflecting across the lumen and into the window.

In some implementations, the rod is adapted to extend through the lumen, alongside the tether, and withdrawing the rod from the lock transitions the lock towards a locked state in which the tab deflects across the lumen and into the window.

There is further provided, in accordance with some implementations, a method (e.g., useable with or for use with a heart of a subject), the method including using a cutter, transluminally pushing a lock distally over and along a tether toward the heart, the lock including: a tubular wall circumscribing a lumen through which the tether extends; a window, cut out of the wall; and a tab, cut from the wall, and positioned opposite the window.

In some implementations, the method further comprises subsequently withdrawing an obstructing rod from the lock such that the tab responsively deflects across the lumen and into the window, thereby locking the lock to the tether.

In some implementations, the method further comprises subsequently cutting the tether by advancing an overtube over and along the cutter, such that the overtube squeezes a pair of cutting edges defined by the cutter towards each other.

In some implementations, the method further comprises subsequently, withdrawing the cutter from the heart.

In some implementations, pushing the lock distally over and along the tether includes pushing the lock over and along the tether while the rod extends through the lock.

In some implementations, pushing the lock distally over and along the tether includes pushing the lock over and along the tether while the rod extends distally out of the cutter and through the lock.

In some implementations, withdrawing the rod from the lock includes withdrawing the rod proximally into the cutter.

In accordance with some implementations, a method includes implanting an implant along a tissue of a heart of a subject (e.g., a living subject, a simulation, etc.) by anchoring to the tissue a series of anchors of the implant.

In some implementations, the anchors are slidably coupled to a tether of the implant.

In some implementations, the method further comprises subsequently pulling the tether through a lock of a head of an anchor of the series to form a loop of the tether.

In some implementations, the method further comprises subsequently locking the lock to the loop.

In some implementations, pulling the loop of the tether through the lock includes grasping the tether and pulling the tether into the lock.

In some implementations, locking the lock to the loop includes withdrawing an unlocking tool from the lock, such that the lock responsively transitions towards a locked state.

In some implementations, the method further includes unlocking the lock prior to pulling the tether through the lock.

The above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

There is further provided, in accordance with some implementations, a system (e.g., useable with or for use with a tether) includes an anchor.

In some implementations, the anchor has an anchor head and a tissue-engaging element extending distally away from the head so as to define an anchor axis of the anchor.

In some implementations, the anchor head defines a channel that extends through the head transverse to the anchor axis, and that is dimensioned to be threaded onto the tether so as to be slidable over and along the tether, and includes a lock, in communication with the channel.

In some implementations, the anchor head defines a proximal opening that provides access to the channel and the lock.

In some implementations, the system comprises a tool, including a grasper configured to advance through the lock (e.g., via the opening) to the channel.

In some implementations, the system includes the tether, threaded through the channel, and the grasper is configured to grasp the tether within the channel, and to form the tether into a loop by pulling the tether proximally through the lock.

In some implementations, the lock is configured to lock onto the loop, responsively to withdrawing the tool from the lock.

In some implementations, the tool includes a tube, the grasper configured to extend through the tube. In some implementations, the grasper is configured to pull the loop into the tube.

In some implementations, the grasper is configured to release the loop once the lock has locked onto the loop.

In some implementations, the tube is configured to unlock the lock by applying an unlocking force to the lock.

In some implementations, the lock is biased to lock in the absence of the unlocking force.

In some implementations, the tube is configured to access the lock via the opening.

In some implementations, the lock includes a first locking arm and a second locking arm, each of the locking arms being hingedly coupled to the head.

In some implementations, the tube is configured to transition the lock toward the unlocked state by pushing against the locking arms in a manner that causes each of the locking arms to pivot away from each other.

In some implementations, the lock is configured to, responsively to withdrawal of the tube from the head, lock onto the loop by the locking arms pivoting towards each other.

In accordance with some implementations, a system and/or an apparatus (e.g., useable with or for use with a tether) includes a lock that includes an outer tube and an inner tube that is positioned coaxially within the outer tube.

In some implementations, the inner tube is fixed to the outer tube at an attachment point.

In some implementations, the inner tube is shaped to define a lumen along a longitudinal axis of the lock, the lumen dimensioned to receive the tether therethrough.

In some implementations, the inner tube is axially compressible towards the attachment point in a manner that causes the inner tube to clamp to the tether within the lumen.

In some implementations, the inner tube is deformable in a manner in which axially compressing the inner tube towards the attachment point causes the inner tube to bow inwardly against the tether.

In some implementations, the inner tube has a first end and a second end, and the inner tube is axially compressible towards the attachment point by moving the second end towards the first end.

In some implementations, the inner tube, at each of the first end and the second end thereof, is annular.

In some implementations, the attachment point is at the first end.

In some implementations, the inner tube is axially compressible towards the attachment point by applying torque to the second end.

In some implementations, the inner tube defines a helix having a series of turns, and application of the torque reduces a pitch of the helix.

In some implementations, the helix is a double helix.

In some implementations, the inner tube is axially compressible towards the attachment point by pushing the second end towards the first end.

In some implementations, the lumen is disposed along the longitudinal axis of the lock, and the inner tube is axially compressible towards the attachment point by pushing one end of the inner tube along the longitudinal axis towards another end of the inner tube.

In some implementations, the lock has an unlocked state in which the lock is slidable along the tether, and/or the lock is transitionable towards a locked state by axially compressing the inner tube.

In some implementations, the lock is transitionable towards the locked state by moving the second end into the outer tube.

In some implementations, in the unlocked state of the lock, the second end is disposed outside of the outer tube, and/or in the locked state of the lock, the second end is disposed inside the outer tube.

In some implementations, the lock includes a snap-fit mechanism between the inner tube and the outer tube, and the inner tube is axially compressible towards the attachment point until the inner tube snap-fits to the outer tube, the snap-fit preventing axial decompression of the inner tube.

In some implementations, the inner tube defines a window, and the outer tube defines a tongue adapted to extend into the window, and the inner tube is axially compressible towards the attachment point until the tongue extends into the window.

In some implementations, the outer tube defines a window, and the inner tube defines a tongue adapted to extend into the window, and the inner tube is axially compressible towards the attachment point until the tongue extends into the window.

In some implementations, the inner tube is shaped to define an hour-glass form having a first bulb, a second bulb, and a waist therebetween, and the inner tube is axially compressible towards the attachment point in a manner that causes the waist to clamp to the tether.

In some implementations, the inner tube is axially compressible towards the attachment point by pushing the first bulb towards the second bulb.

In some implementations, each of the first bulb and the second bulb is annular.

In accordance with some implementations, a method (e.g., useable with or for use with a tether) includes transluminally advancing a lock to a heart of a subject (e.g., a living subject, a simulation, etc.) over and along the tether, the lock including an inner tube, positioned coaxially within an outer tube and fixed to the outer tube at an attachment point, the tether extending through a lumen defined by the inner tube.

In some implementations, the method includes, at the heart, locking the lock to the tether by axially compressing the inner tube towards the attachment point such that the inner tube clamps to the tether within the lumen.

In some implementations, compressing the inner tube towards the attachment point such that the inner tube clamps to the tether includes compressing the inner tube towards the attachment point such that a diameter of the inner tube narrows.

In some implementations, axially compressing the inner tube towards the attachment point includes pushing the inner tube further into the outer tube.

In some implementations, the inner tube has a first end and a second end, and axially compressing the inner tube towards the attachment point includes axially compressing the inner tube by pushing the second end towards the first end.

In some implementations, the attachment point is disposed at the first end.

In some implementations, transluminally advancing the lock includes transluminally advancing the lock while the second end is disposed outside of the outer tube, and/or pushing the second end towards the first end includes pushing the second end into the outer tube.

In some implementations, the lumen is disposed along a longitudinal axis of the lock, and axially compressing the inner tube towards the attachment point includes axially compressing the inner tube by axially pushing one end of the inner tube along the longitudinal axis towards another end of the inner tube.

In some implementations, axially compressing the inner tube toward the attachment point includes axially compressing the inner tube toward the attachment point until the inner tube snap-fits to the outer tube, the snap-fit preventing axial decompression of the inner tube.

In some implementations, the lock defines a window and a tongue, and axially compressing the inner tube toward the attachment point until the inner tube snap-fits to the outer tube includes axially compressing the inner tube toward the attachment point until the tongue protrudes into the window.

In some implementations, axially compressing the inner tube toward the attachment point until the tongue protrudes into the window includes axially compressing the inner tube toward the attachment point until the tongue becomes aligned with the window and, responsively to becoming aligned with the window, automatically protrudes into the window.

In some implementations, axially compressing the inner tube toward the attachment point until the tongue becomes aligned with the window includes axially compressing the inner tube toward the attachment point such that the tongue moves into axial alignment with the window.

In some implementations, axially compressing the inner tube toward the attachment point until the tongue becomes aligned with the window includes axially compressing the inner tube toward the attachment point by twisting the inner tube towards the attachment point until the tongue moves into rotational alignment with the window.

In some implementations, the outer tube defines the tongue, and the inner tube defines the window, and axially compressing the inner tube toward the attachment point until the tongue extends into the window includes axially compressing the inner tube toward the attachment point until the tongue of the outer tube extends into the window of the inner tube.

In some implementations, axially compressing the inner tube towards the attachment point such that the inner tube clamps to the tether includes axially compressing the inner tube towards the attachment point such that part of the inner tube bows medially against the tether.

In some implementations, axially compressing the inner tube towards the attachment point such that the part of the inner tube bows medially against the tether includes axially compressing the inner tube towards the attachment point such that the inner tube acquires an hourglass shape having a waist that clamps to the tether.

In some implementations, the inner tube has a first end-portion and a second-end portion, and is shaped to define multiple struts extending between the first end-portion and the second-end portion, and axially compressing the inner tube towards the attachment point such that the inner tube clamps to the tether includes axially compressing the inner tube towards the attachment point such that the struts bow medially against the tether.

In some implementations, each of the struts has an hour-glass shape having a first bulb, a second bulb, and a waist therebetween, and axially compressing the inner tube towards the attachment point such that the struts bow medially against the tether includes axially compressing the inner tube towards the attachment point such that the waist of each of the struts clamps to the tether.

In some implementations, axially compressing the inner tube towards the attachment point such that the inner tube clamps to the tether includes axially compressing the inner tube towards the attachment point in a manner in which, for each of the struts, the first bulb moves towards the second bulb.

In some implementations, axially compressing the inner tube towards the attachment point includes axially compressing the inner tube towards the attachment point such that the inner tube twists inwardly against the tether.

In some implementations, the inner tube is shaped to define a helix that has a series of turns and a pitch, and axially compressing the inner tube towards the attachment point such that the inner tube twists inwardly against the tether includes reducing the pitch by twisting the inner tube towards the attachment point.

In some implementations, the helix is a double-helix, and reducing the pitch includes reducing the pitch of the double-helix.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

### BRIEF DESCRIPTION OF THE DRAWINGS

FIGS. 1A-B and 2A-B are schematic illustrations of a system, and techniques for use therewith, in accordance with some implementations;

FIGS. 3-4 are schematic illustrations of examples of implantation techniques, in accordance with some implementations;

FIGS. 5A-B and 6 are schematic illustrations showing systems, apparatuses, and techniques for contracting tissue differentially along the path of an implant by application of tension-modifiers to the implant, in accordance with some implementations;

FIGS. 7A-F are schematic illustrations of a system and techniques for use therewith, in accordance with some implementations;

FIGS. 8A-C, 9A-B, 10A-B, 11A-G, 12A-C, 13A-B, 14A-B, 15A-C, and 16A-D are schematic illustrations of systems and techniques for use therewith, in accordance with some implementations;

FIGS. 17A-E and 18 are schematic illustrations of a system and techniques for use therewith, in accordance with some implementations;

FIGS. 19A-G are schematic illustrations of a system and techniques for tensioning an implant of the system, in accordance with some implementations;

FIGS. 20A-C, 21, 22A-B, and 23A-E are schematic illustrations of system or apparatus for use with a tether, in accordance with some implementations; and

FIGS. 24A-B, 25, and 26A-B show a variety of locks that are adapted to clamp onto a tether of an implant, in accordance with some implementations.

### DETAILED DESCRIPTION

Systems, apparatuses, methods, devices, etc. for inhibiting tissue growth on an implantable sheet are described herein. In some implementations, systems, apparatuses, methods, devices, etc. include an apparatus that comprises a sheet constructed of a multi-layered structure and/or implants that incorporate the sheet. The systems, apparatuses, methods, devices, etc. can be configured to inhibit tissue growth thereon. Various examples of apparatuses and/or methods for coating an implant inhibiting tissue growth thereon are described. In some implementations, an implant that incorporates the sheet is configured to support, and/or to guide movement of a leaflet of a valve of a heart of a subject (e.g., a living subject, a simulation, etc.). An example of where such an implant can be helpful is when used at the posterior leaflet of a mitral valve experiencing flail, prolapse, rigidity, and/or another issue resulting in regurgitations.

The described systems, apparatuses, methods, devices, etc. should not be construed as limiting in any way. Instead, the present disclosure is directed toward all novel and nonobvious features and aspects of the various disclosed coating procedures and applications, alone and in various combinations and sub-combinations with one another. The disclosed systems, apparatuses, methods, devices, etc. are not limited to any specific aspect, feature, or combination thereof, nor do the disclosed systems, apparatuses, methods, devices, etc. require that any one or more specific advantages be present, or problems be solved. Further, the techniques, methods, operations, steps, etc. described or suggested herein can be performed on a subject (e.g., a living animal or on a non-living simulation, such as on a cadaver, cadaver heart, simulator, etc.).

Various implementations of systems, devices, examples of prosthetic implants, etc. can be coated using the coating methods disclosed herein, and any combination of the described features, components, and options can be made unless specifically excluded. For example, various polymers inhibiting tissue growth thereon can be used with any appropriate methods for binding them to an apparatus, even if a specific combination of heat and/or pressure is not explicitly described. Likewise, the different constructions and features of devices can be mixed and matched, such as by combining any sheet with any form of frame, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

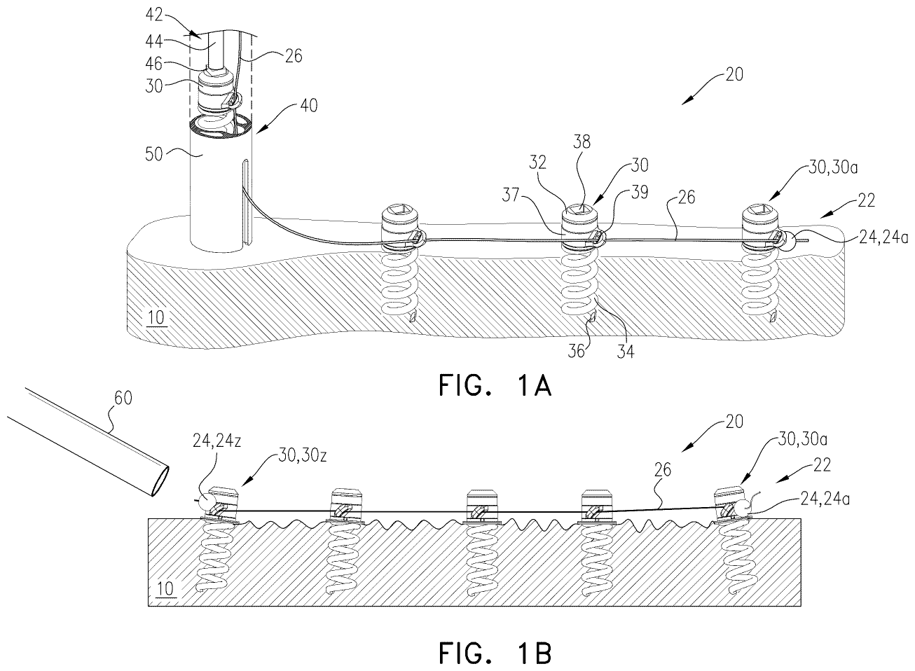

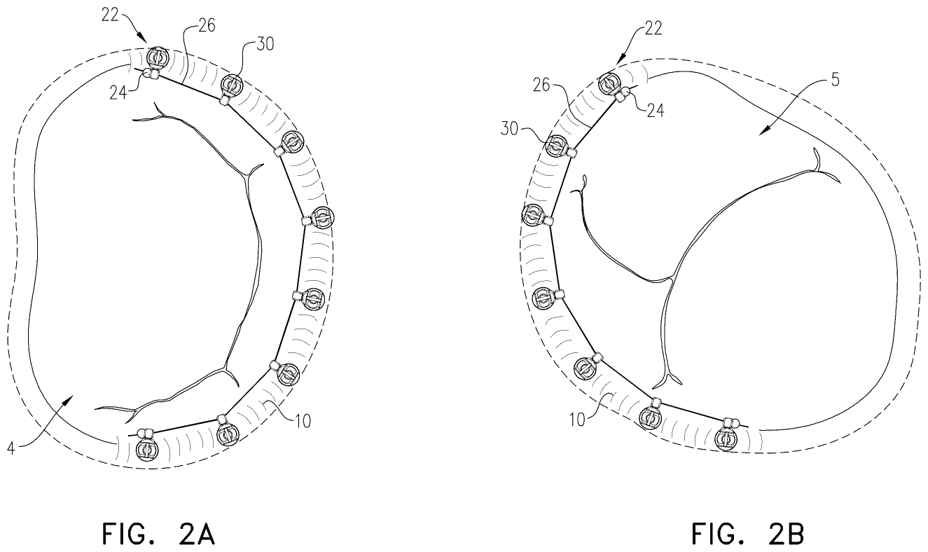

Reference is made to FIGS. 1A-B and 2A-B, which are schematic illustrations of a system 20 and techniques for implanting an implant 22 of the system, in accordance with some implementations. System 20 is a tissue-adjustment system and can be used for adjusting a dimension of a tissue 10, such as a tissue of the heart of a subject. For example, system 20 can be an annuloplasty system, and implant 22 can be an annuloplasty structure (e.g., an annuloplasty ring or band) for implanting at (e.g., along) an annulus of a valve of the heart. Implant 22 can comprise multiple anchors 30 and a tether 26 (e.g., a contracting member) to which the anchors are slidably coupled, e.g., by being threaded on the tether.

Tether 26 is an elongate member (e.g., a wire, a cable, a line, a suture, a filament, a ribbon, etc.), and is typically flexible. Tether 26 can comprise a metal, a polymer, and/or a natural fiber. In some implementations, tether 26 comprises a suture. In some implementations, tether 26 comprises a superelastic material. Tether 26 can comprise nitinol, ePTFE, PTFE, polyester, stainless steel, and/or cobalt chrome.

Each anchor 30 comprises a tissue-engaging element 34 (e.g., one or more of a helix, screw, point, hook, barb, dart, arm, staple, sharpened portion, clip, pledget, etc.). In some implementations each anchor can optionally also include a head 32, with the tissue-engaging element extending distally away from the head. In some implementations, tissue-engaging element 34 has a sharpened distal tip 36, and is configured to be driven (e.g., screwed, pushed, etc.) into tissue of the subject. Tissue-engaging element 34 can define a central longitudinal axis of anchor 30. For example, and as shown, tissue-engaging element 34 can be helical, with its screw axis being the central longitudinal axis of the anchor. Alternatively or additionally, anchor 30 can comprise another type of tissue-engaging element, such as a dart or a staple. In some implementations, the tissue-engaging element can include barbs or barbed portions to hold the tissue-engaging element in tissue.

In some implementations, a head 32 can be coupled to proximal end of tissue-engaging element 34, and can comprise a driver interface 38, and can also comprise an eyelet 39. Driver interface 38 can be rigidly coupled to tissue-engaging element 34. In some implementations, and as shown, driver interface 38 is disposed on the central longitudinal axis of anchor 30. In some implementations, and as shown, eyelet 39 is disposed laterally from the central longitudinal axis of anchor 30. In some implementations, an internal diameter of eyelet 39 is 0.25-0.75 mm.

Tether 26 can comprise (e.g., consist substantially of) a wire, a cable, a suture, a cord, a yarn, a thread, a line, and a ribbon. Tether 26 can comprise (e.g., consist substantially of) a metal, a polymer, a synthetic fiber, a semisynthetic fiber, and/or a natural fiber. Tether 26 can be circular, oval, rectangular, or another shape in cross-section. In some implementations, tether 26 is 0.1-0.6 mm (e.g., 0.18-0.3 mm) thick.

System 20 can comprise, in addition to implant 22, a delivery tool 40 for percutaneous (e.g., transluminal, such as transfemoral) implantation of the implant. Tool 40 comprises a flexible anchor driver 42 that is configured to reversibly engage driver interface 38 of each anchor 30. Via this engagement, driver 42 is configured to drive (e.g., screw) tissue-engaging element 34 into tissue—e.g., by rotating driver interface 38. For example, and as shown, driver 42 can comprise an elongate and flexible shaft 44, and a driver head 46 that is coupled to a distal end of the shaft and that is configured to reversibly engage driver interface 38. In some implementations, and as shown, tool 40 further comprises a flexible tube 50 (e.g., a transluminal catheter) via which each anchor 30 is advanceable to the tissue 10 to which the anchor is to be anchored.

In some implementations, implant 22, delivery tool 40, and/or system 20 as a whole, can share features with one or more implants, delivery tools, and/or systems described in one or more of the following references, each of which is incorporated herein by reference:
U.S. patent application Ser. No. 14/437,373 to Sheps et al., which published as US 2015/0272734
U.S. patent application Ser. No. 15/782,687 to Iflah et al., which published as US 2018/0049875
International Patent Application PCT/IB2020/060044 to Kasher et al., which published as WO 2021/084407
International Patent Application PCT/IB2021/058665 to Halabi et al., which published as WO 2022/064401
International Patent Application PCT/IB2022/051099 to Shafigh et al., which published as WO 2022/172149

Anchor 30 (e.g., eyelet 39 thereof) is configured to facilitate sliding of the anchor along tether 26. In some implementations, the anchor (e.g., the eyelet) is configured to facilitate such sliding both (i) while the anchor (e.g., its central longitudinal axis) is aligned with the tether, e.g., in order to facilitate advancement of the anchor along the tether to the heart—and (ii) while the anchor (e.g., its central longitudinal axis) is oriented orthogonally to the tether, e.g., in order to subsequently facilitate contraction of the implant and thereby of the tissue to which the implant is anchored. This can be achieved at least partly due to the shape and dimensions of eyelet 39, which can be as described in one or more of the references cited hereinabove, mutatis mutandis.

In some implementations, eyelet 39 is revolvable around the central longitudinal axis of anchor 30, e.g., by being mounted on a cuff 37 that is rotatably mounted. In some implementations, despite its revolvability around the central longitudinal axis of anchor 30, cyelet 39 is at a fixed angular disposition with respect to the central longitudinal axis, e.g., by being rigidly mounted on cuff 37, which is rotationally but non-deflectably mounted.

FIG. 1A shows three anchors 30 having been anchored to tissue 10, and driver 42 delivering a fourth anchor via tube 50. Each anchor 30 can be delivered while tether 26 extends through eyelet 39 while generally parallel to the central longitudinal axis of the anchor, e.g., as shown for the fourth anchor in FIG. 1A. For anchors that have been previously anchored (e.g., the right-most three anchors in FIG. 1A), tether 26 has become oriented laterally with respect to the anchors (e.g., orthogonally to the central longitudinal axis of the anchor).

After a desired number of anchors 30 have been anchored, tension is applied to tether 26 (e.g., by sliding the tether with respect to one or more of anchors 30), and is locked into the tether (e.g., by inhibiting further and/or reverse sliding of the tether with respect to one or more of the anchors). For example, FIG. 1B shows an adjustment tool 60 having been introduced (e.g., over and along a proximal portion of tether 26), having applied tension to tether 26, and having locked the tension by locking a lock (e.g., a stopper) 24 to the tether at the last anchor 30 to have been anchored. This last anchor 30 to have been anchored is labeled 30z, and can be considered to be a proximal-most anchor of implant 22.

The tensioning of tether 26 draws anchors 30 closer together, thereby contracting tissue 10 to which the anchors are anchored. In order to facilitate tensioning of tether 26, adjustment tool 60 can provide a reference force (e.g., pressing against the last anchor 30 to have been anchored) while the tether (e.g., a proximal end of the tether) is pulled proximally. In the example shown, adjustment tool 60 has also trimmed excess tether 26 and is being withdrawn from the heart. Adjustment tool 60 can be a component of system 20. In some implementations, adjustment tool 60, lock 24, and/or techniques for tensioning, locking, and/or trimming tether 26 can be as described, mutatis mutandis, and/or can share features with one or more of those described, mutatis mutandis, in one or more of the following references, each of which is incorporated herein by reference:
U.S. patent application Ser. No. 16/534,875 to Brauon et al., filed Aug. 7, 2019, which published as US 2020/0015971
International Patent Application PCT/IB2020/060044 to Kasher et al., which published as WO 2021/084407
International Patent Application PCT/IB2022/051099 to Shafigh et al., which published as WO 2022/172149

The coupling of tether 26 to the first anchor 30 to be anchored is such that the pulling of tether 26 in order to tension the tether does not cause the tether (e.g., a first end thereof) to become released (e.g., slid out) from the first anchor to have been anchored. This first anchor 30 to be anchored is labeled 30a, and can be considered to be a distalmost anchor of implant 22. In some implementations, and as shown, a lock (e.g., a stopper) 24 can be locked to tether 26 at anchor 32a, e.g., with anchor 30a being identical to the other anchors 30 of implant 22. This lock 24 is labeled 24a, while the lock at anchor 30z is labeled 24z. In some implementations, lock 24a is identical to lock 24z. In some implementations, locks 24a and 24z are different from each other, e.g., due to their slightly different roles. In some implementations, anchor 30a can be fixedly attached to tether 26, e.g., with anchor 30a comprising a specific feature or component (e.g., a lock) to facilitate such attachment. In some implementations, implant 22 does not comprise a lock 24a that is discrete from anchor 30a.

For simplicity, FIGS. 1A and 1B show implant 22 in a linear configuration. However, for annuloplasty, implant 22 is often implanted in a curve (or even a complete ring) around the valve annulus, such that the contraction reduces the size of the valve annulus, improving coaptation of the valve leaflets. FIG. 2A shows an example in which implant 22 is implanted in a curve around the annulus of a mitral valve 4 of a heart of a subject, e.g., with tissue 10 being tissue of the mitral annulus. FIG. 2B shows an example in which implant 22 is implanted in a curve around the annulus of a tricuspid valve 5 of a heart of a subject, e.g., with tissue 10 being tissue of the tricuspid annulus.

In the above description of implant 22, with reference made to FIGS. 1A-2B, tension along the entirety of tether 26, and thereby contraction of tissue 10 between first/distalmost anchor 30a and last/proximal-most anchor 30z (e.g., along the entirety of the implant), is substantially uniform. Hereinbelow are described, inter alia, systems, implants, and techniques for contracting tissue differentially along an implant (e.g., by differing tension along an implant and/or a tether), and/or for contracting tissue of an annulus of a heart valve differentially along the annulus.

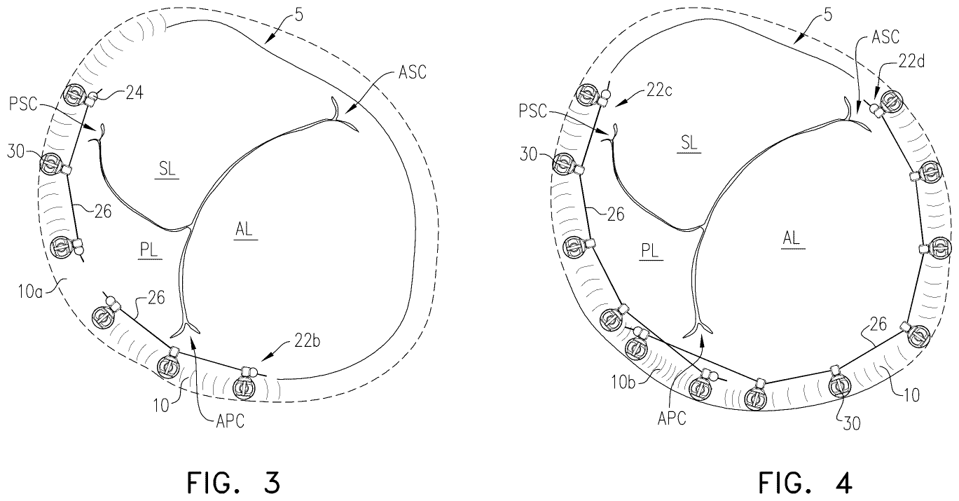

Reference is made to FIGS. 3-4, which are schematic illustrations of example implantations of implant 22, in accordance with some implementations. FIG. 3 shows a first implant 22a having been anchored along a first portion of the annulus, and a second implant 22b having been anchored along a second portion of the annulus, leaving a space between the first and second portions of the annulus. FIG. 4 shows a first implant 22c having been anchored along a first portion of the annulus, and a second implant 22d having been anchored along a second portion of the annulus, with an overlap between the first and second portions of the annulus.

Implants 22a, 22b, 22c, and 22d can be as described for implant 22, and can be considered to be variants of implant 22. In some implementations, these variants are identical to implant 22. In some implementations, these variants are identical to implant 22 except for their length (e.g., the length of their tethers 26) and/or the number of anchors 30 that they comprise.

In the example shown in FIG. 3, implants 22a and 22b are anchored along the annulus of tricuspid valve 5, with the first portion of the annulus (along which first implant 22a is anchored) extending between the roots of septal leaflet SL and posterior leaflet PL, including posteroseptal commissure PSC; and the second portion of the annulus (along which second implant 22b is anchored) extending between the roots of anterior leaflet AL and posterior leaflet PL, including anteroposterior commissure APC. After implantation and contraction of implants 22a and 22b, tissue 10a (e.g., tissue of the annulus) in the space between the first and second portions of the annulus (e.g., between implants 22a and 22b) may remain uncontracted. It is to be understood that other similar arrangements are possible in which a gap is left between two annuloplasty implants, and that similar arrangements are applicable to other valves of the heart, such as the mitral valve, mutatis mutandis.

In the example shown in FIG. 4, implants 22c and 22d are anchored along the annulus of tricuspid valve 5, with the first portion of the annulus (along which first implant 22c is anchored) extending from posteroseptal commissure PSC, along the root of posterior leaflet PL, past anteroposterior commissure APC, to the root of anterior leaflet AL; and the second portion of the annulus (along which second implant 22d is anchored) extending from anteroseptal commissure ASC, along the root of anterior leaflet AL, past anteroposterior commissure APC, to the root of posterior leaflet PL. After implantation and contraction of implants 22c and 22d, tissue 10b (e.g., tissue of the annulus) where the first and second portions of the annulus overlap (e.g., where implants 22c and 22d overlap) may experience increased contraction and/or contractive force compared to portions of the annulus at which only a single implant is present. In the example shown, tissue 10b is at anteroposterior commissure APC. It is to be understood that other similar arrangements are possible in which a gap is left between two annuloplasty implants, and that similar arrangements are applicable to other valves of the heart, such as the mitral valve, mutatis mutandis.

Reference is again made to FIGS. 3-4. Such use of multiple implants technique may provide greater control over the contraction of the annulus, e.g., by implanting an implant only along portions of the annulus along which contraction is desired, and/or by implanting more than one implant along a portion of the annulus at which additional contraction and/or contractive strength are desired. It is to be noted that, in some implementations, the techniques described with reference to FIG. 3 can be combined with those described with reference to FIG. 4, mutatis mutandis.

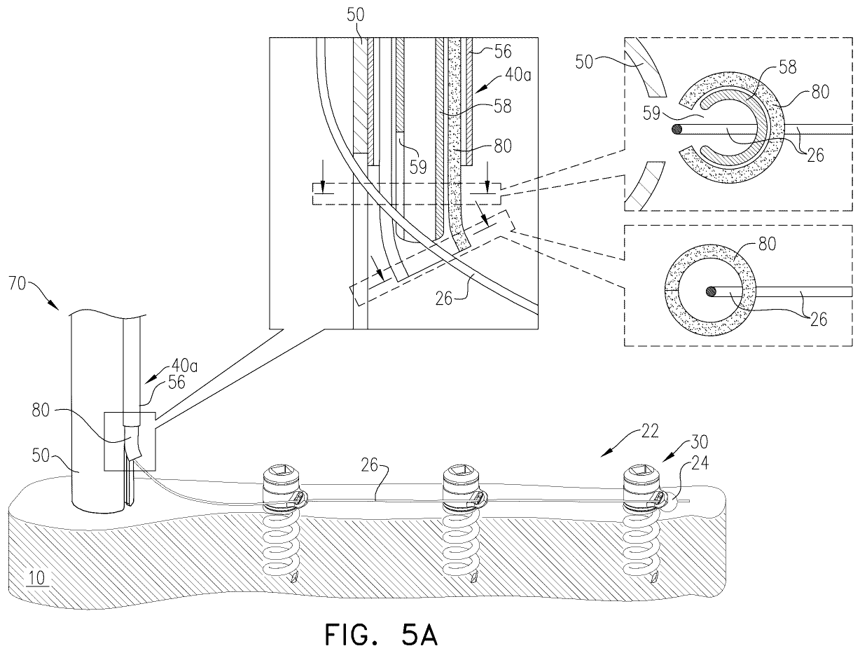

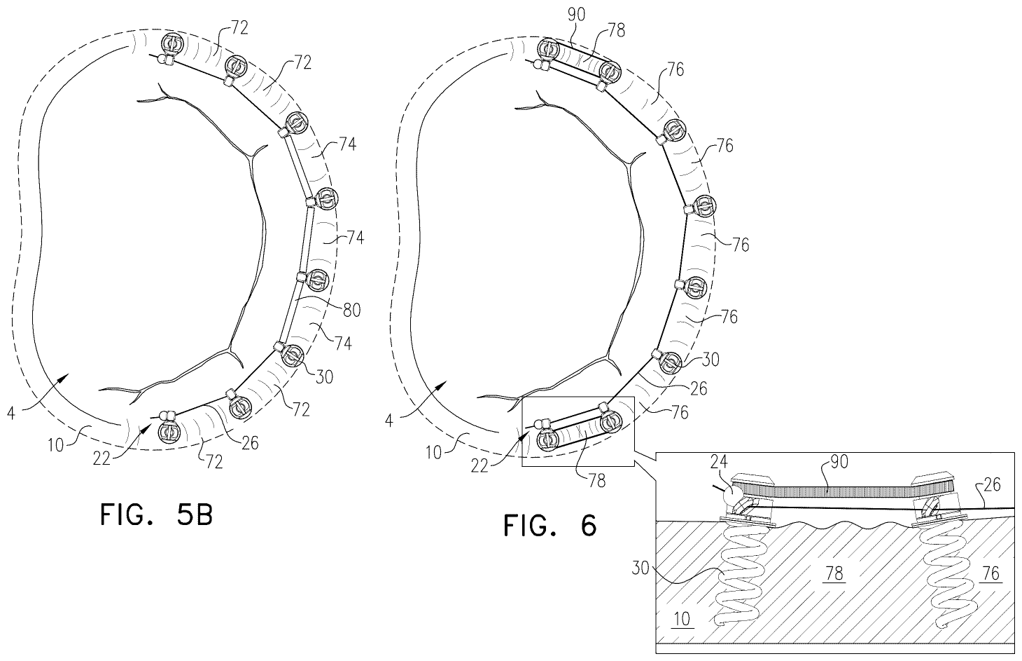

Reference is now made to FIGS. 5A-B and 6, which are schematic illustrations showing systems, apparatuses, and techniques for contracting tissue differentially along the path of implant 22 by application of tension-modifiers to the implant, in accordance with some implementations. FIGS. 5A-B show the use of tension-modifiers that are in the form of spacers 80, and FIG. 6 shows the use of tension-modifiers that are in the form of connectors 90.

FIG. 5A shows a system 70 that includes implant 22, and a delivery tool 40a, which can be as described for delivery tool 40, except that it further comprises an applicator 56. Applicator 56 is configured to intracardially connect spacers 80 to implant 22, by intracardially connecting the spacers to tether 26 (e.g., intracardially threading the spacers onto the tether). Each spacer 80, or more than one spacer, can be connected to tether 26 between a pair of adjacent anchors 30. For example, FIG. 5A shows applicator 56 connecting a spacer 80 to tether 26 after a third anchor has been anchored to tissue, and prior to a fourth anchor being anchored to the tissue. FIG. 5B shows implant 22 having been implanted, with this same spacer, as well as two more spacers, connected to tether 26, each spacer disposed between respective pairs of adjacent anchors 30.

In some implementations, each spacer 80 is elongate and, as shown, can be substantially tubular, at least at rest. Applicator 56 can be configured to intracardially connect each spacer 80 to tether 26 while the spacer is constrained in an open state in which the spacer has a substantially C-shaped cross section. In some implementations, the entirety of the spacer is constrained in this open state concurrently. In some implementations, each region of the spacer is transiently moved into the open state as it is introduced onto the tether, such that an open region of the spacer passes along the spacer like a wave as the spacer is introduced onto the tether. For example, for applications in which the spacer is advanced distally onto the tether, this wave may move from the distal end to the proximal end of the spacer.

Applicator 56 can be configured to advance spacers 80 out of a secondary lumen disposed laterally from the primary lumen of tube 50. In some implementations, and as shown, applicator 56 can comprise a tube that defines this secondary lumen.

Applicator 56 is controllable from outside of the subject, e.g., via an applicator controller on a handle of delivery tool 40a. For example, the applicator controller can be a button or trigger that, upon being pressed, applies a spacer 80 to tether 26. For example, the applicator controller can be operatively coupled to a pusher of applicator 56, such that operation of the applicator controller causes the pusher to push a spacer 80 distally out/off of the applicator and onto tether 26.

In some implementations, applicator 56 comprises a mandrel 58 that constrains each spacer 80 in its open state. As shown, at least part (e.g., a distal part) of mandrel 58 can be hollow (e.g., tubular or funnel-like). Furthermore, at least a distal part of mandrel 58 (e.g., at least part of the hollow part) can have a lateral slit 59 (e.g., parallel with the axis of the mandrel, or of the applicator as a whole), into which tether 26 may pass. In some implementations, throughout implantation of implant 22, tether 26 remains extended through the slit into the hollow of mandrel 58, and out of the distal end of the mandrel, and as the implant is progressively implanted the tether slides linearly while remaining in this arrangement. In some implementations, mandrel 58 can be advanced over tether 26 (e.g., such that the tether passes through the slit) for the application of each spacer, and subsequently withdrawn until the application of another spacer. In either case, for applications in which mandrel 58 is hollow and has lateral slit 59, while tether 26 is disposed through the slit, the tether can extend into the mandrel via the slit and out of the mandrel via an end opening of the mandrel, e.g., as shown. Thus, while tether 26 is disposed through the slit, spacer 80 can be applied to the tether by pushing the spacer off of the end of mandrel 58. As each region of spacer 80 leaves mandrel 58, it ceases to be constrained in its open state, and thus responsively closes around tether 26. In the resulting closed state of spacer 80, the spacer can be tubular, e.g., with the two open edges of its c-shape meeting edge-to-edge, or wrapping over each other.

Each spacer 80 is configured to inhibit the pair of anchors disposed on either side of the spacer from moving toward each other during contraction of implant 22 (e.g., during tensioning of tether 26), e.g., to limit how close the anchors can become to each other. For example, spacers 80 can have features and/or functionality such as that described, mutatis mutandis, in International Patent Application PCT/IB2020/060044 to Kasher et al., which published as WO 2021/084407, and/or International Patent Application PCT/IB2022/051099 to Shafigh et al., each of which is incorporated herein by reference. Thus, depending on the spacing between the relevant pair of adjacent anchors 30 at the time that spacer 80 is applied, application of the spacer between the pair of adjacent anchors may urge the pair of adjacent anchors away from each other.

FIG. 5B schematically illustrates inter-anchor regions 72 of tissue 10 between pairs of anchors 30 between which a spacer 80 is not disposed. Regions 72 are therefore shown as being contracted, similarly to as described in FIGS. 1A-2B, mutatis mutandis—or even to a greater extent. FIG. 5B also schematically illustrates inter-anchor regions 74 of tissue 10 between pairs of anchors between which a spacer 80 is disposed. Regions 74 are therefore shown as being contracted less than regions 72.

System 70 thereby facilitates control over the degree of contraction that is to be applied to different sections of implant 22, and thereby to different regions of tissue 10 along which the implant is implanted. In the particular example shown, regions 74 are shown as being on the annulus of mitral valve 4 in the vicinity of the P2 scallop of the posterior leaflet, while regions 72 are shown as being in the vicinity of the commissures and/or the P1 and P3 scallops, e.g., so as to provide greater contraction along the anteroposterior axis of the valve and less contraction along the commissure-to-commissure axis of the valve. However, because spacers 80 are added to implant 22 during implantation, the operator (e.g., the physician) may decide intraoperatively whether to apply a spacer between a given pair of anchors (e.g., between the most-recently anchored anchor and the next anchor to be anchored), e.g., based on data (e.g., imaging and/or numerical data) acquired during the procedure. For example, a spacer might be applied between a given pair of anchors in light of a particular anatomical feature and/or a particular observed effect of the implant on the tissue.

FIG. 6 shows implant 22 having been implanted, with two connectors 90 connected to respective pairs of anchors 30, in accordance with some implementations. Each connector 90 is configured to draw the pair of anchors disposed on either side of the connector toward each other, and/or to inhibit the anchors from moving away from each other. Connectors 90 can be applied intracardially. In some implementations, connectors 90 are applied during implantation, such as by using an applicator that is part of a delivery tool, e.g., similarly to as described for applicator 56, mutatis mutandis. In some implementations, connectors 90 are applied after implantation—and optionally after contraction (e.g., an initial contraction) of the implant. For example, once all of anchors 30 of the implant have been drawn toward each other by applying tension to tether 26, the addition of a connector between a pair of anchors may draw the pair of anchors further toward each other, optionally increasing a distance between at least one of the anchors of the pair and another anchor not of the pair.

In the example shown, connector 90 is illustrated as a band that is wrapped around the head of each anchor 30 of the pair (see inset). However, the scope of the present disclosure includes other implementations of connector 90 such as a secondary tether (e.g., alongside tether 26).

In some implementations, and as shown, each connector 90 can be substantially parallel with part of tether 26.

FIG. 6 schematically illustrates inter-anchor regions 76 of tissue 10 between pairs of anchors 30 that are not connected by a connector 90. Regions 76 are therefore shown as being contracted, similarly to as described in FIGS. 1A-2B, mutatis mutandis—or even to a lesser extent. FIG. 6 also schematically illustrates inter-anchor regions 78 of tissue 10 between pairs of anchors that are connected by a connector 90. Regions 78 are therefore shown as being contracted more than regions 72.

A system that utilizes connectors 90 thereby facilitates control over the degree of contraction that is to be applied to different sections of implant 22, and thereby to different regions of tissue 10 along which the implant is implanted. In the particular example shown, regions 76 are shown as being on the annulus of mitral valve 4 primarily in the vicinity of the P2 scallop of the posterior leaflet, while regions 78 are shown as being in the vicinity of the commissures and/or the P1 and P3 scallops, e.g., so as to provide greater contraction along the anteroposterior axis of the valve and less contraction along the commissure-to-commissure axis of the valve. However, in some implementations in which connectors 90 are added to implant 22 during implantation, the operator (e.g., the physician) may decide intraoperatively whether to apply a connector between a given pair of anchors (e.g., between the most-recently anchored anchor and the next anchor to be anchored), e.g., based on data (e.g., imaging and/or numerical data) acquired during the procedure. For example, a connector might be applied between a given pair of anchors in light of a particular anatomical feature and/or a particular observed effect of the implant on the tissue.

It is to be noted that the tension-modifiers described hereinabove may be introduced subsequently to the original implantation of the implant, e.g., as a result of post-implantation observations (e.g., using imaging techniques). In accordance with some implementations, a method that comprises (1) identifying a subject in whom an implant has been implanted along a tissue of a heart of the subject, the implant including a series of anchors slidably coupled to a tether, and the implant having been implanted by anchoring the series of anchors to the tissue; and (2) in response to the identifying, adjusting tension on the tether by applying a tension-modifier between a pair of adjacent anchors of the series.

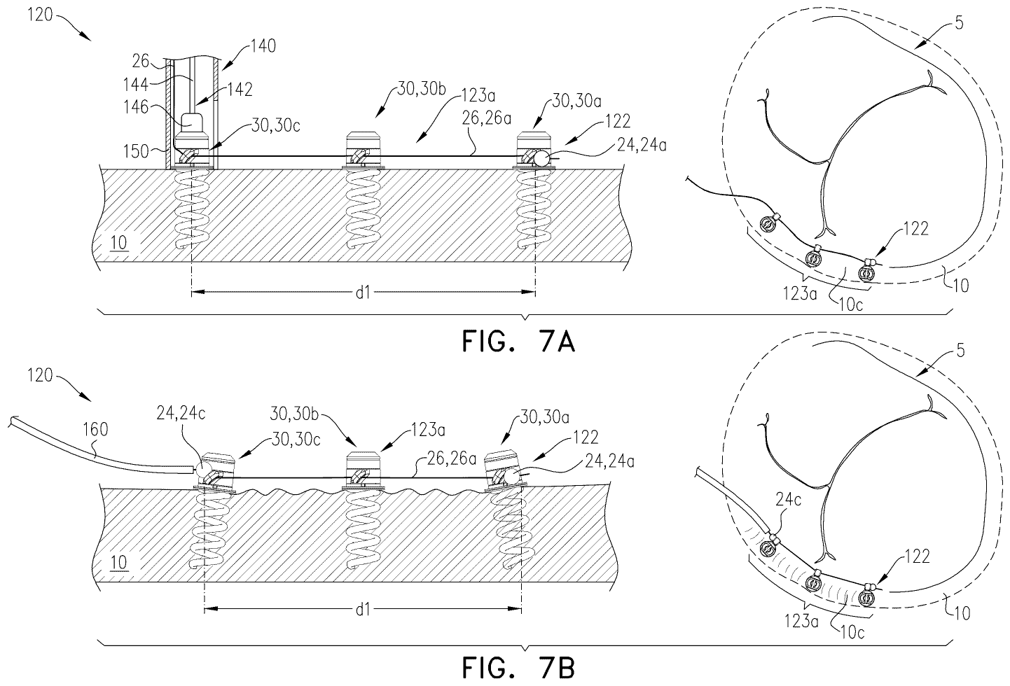

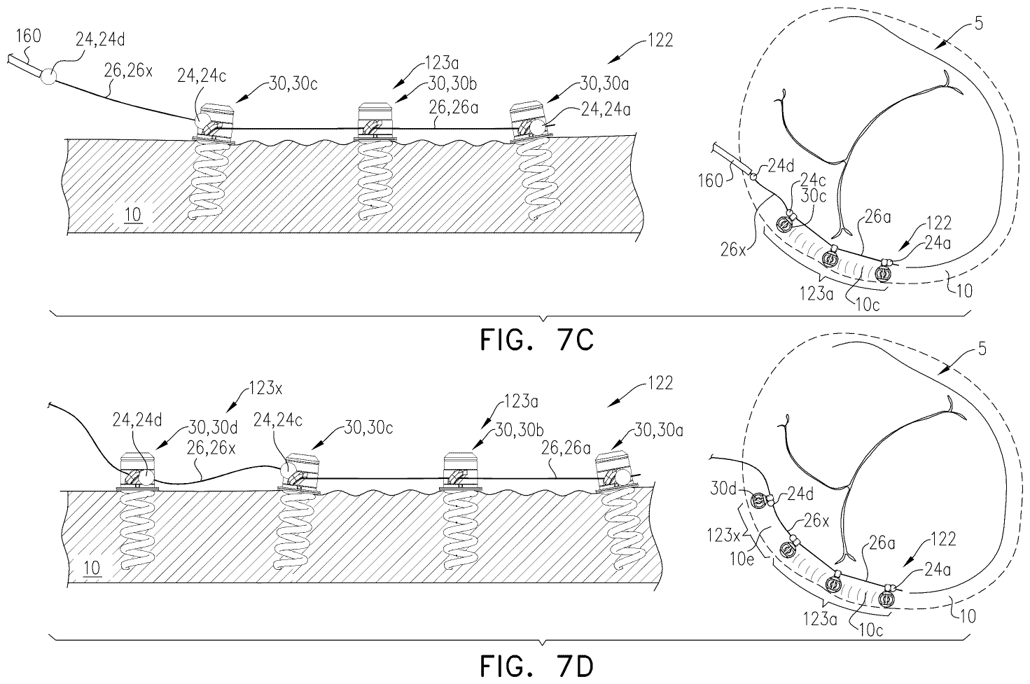

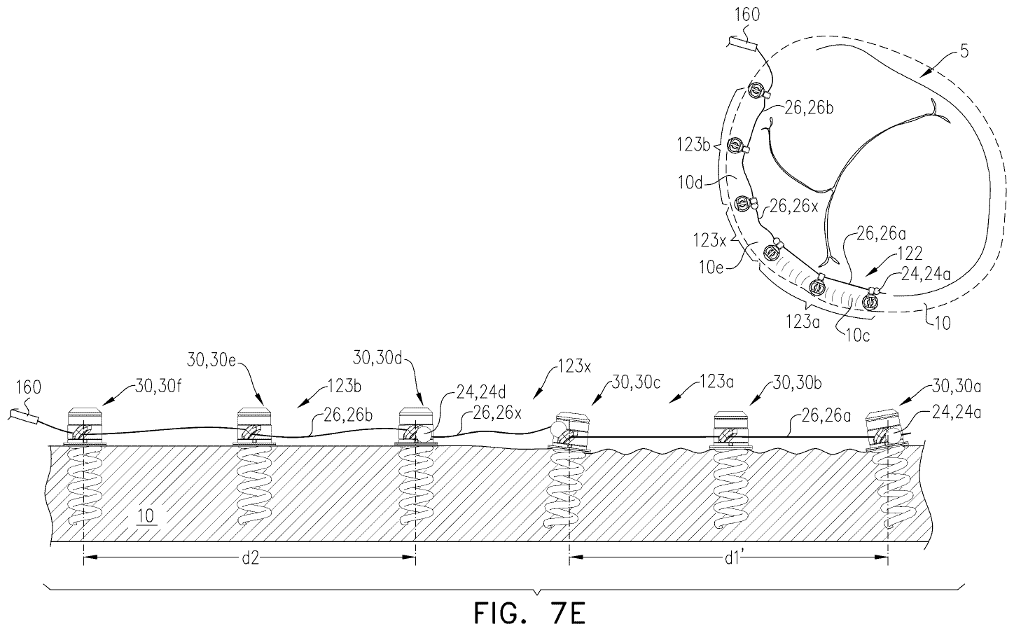

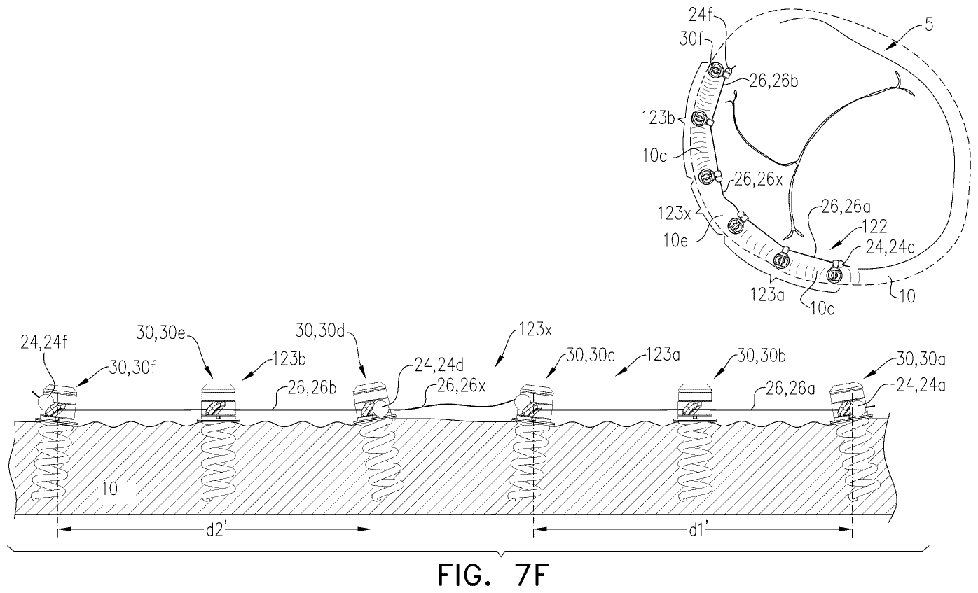

Reference is made to FIGS. 7A-F, which are schematic illustrations of a system 120 and techniques for implanting an implant 122 of the system, in accordance with some implementations. System 120 is a tissue-adjustment system and can be used for adjusting a dimension of a tissue 10, such as a tissue of the heart of a subject. For example, system 120 can be an annuloplasty system, and implant 122 can be an annuloplasty structure (e.g., an annuloplasty ring or band) for implanting at (e.g., along) an annulus of a valve of the heart. Implant 122 can comprise multiple anchors 30 and tether 26 (e.g., a contracting member) to which the anchors are slidably coupled, e.g., by being threaded on the tether. Thus, in some implementations, implant 122 and implantation thereof are as described for implant 22, mutatis mutandis, except where noted. Furthermore, in some implementations implant 122 can be considered to be a variant of implant 22.

System 120 can comprise, in addition to implant 122, a delivery tool 140 for percutaneous (e.g., transluminal, such as transfemoral) implantation of the implant. Tool 40 can comprise a flexible anchor driver 142 that is configured to reversibly engage driver interface 38 of each anchor 30. Via this engagement, driver 142 is configured to drive (e.g., screw) tissue-engaging element 34 into tissue, e.g., by rotating driver interface 38. For example, and as shown, driver 142 can comprise an elongate and flexible shaft 144, and a driver head 146 that is coupled to a distal end of the shaft and that is configured to reversibly engage driver interface 38. In some implementations, and as shown, tool 140 further comprises a flexible tube 150 (e.g., a transluminal catheter) via which each anchor 30 is advanceable to the tissue 10 to which the anchor is to be anchored. Thus, in some implementations, tool 140 is as described for tool 40, mutatis mutandis, except where noted. Furthermore, in some implementations tool 140 can be considered to be a variant of tool 40.

FIGS. 7A-F show implant 122 being implanted along tissue 10 (e.g., along the annulus of valve 5) such that the implant defines an elongate path along the tissue. FIG. 7A shows a first section 123a of implant 122 having been anchored along a first portion 10c of the path, by some anchors 30 of the implant having been anchored to tissue 10 by tool 140, e.g., as described hereinabove for implant 22 and tool 40, mutatis mutandis. In the particular example shown, first section 123a comprises three anchors 30, e.g., first anchor 30a (described hereinabove), a second anchor 30b, and a third anchor 30c. That is, in the particular example shown, anchor 30a is the distalmost anchor of first section 123a (and of implant 122 as a whole), and anchor 30c is the proximal-most anchor of first section 123a. However, more or fewer anchors can be used. More generally, first section 123a can be defined as being between the distalmost anchor of the first section (anchor 30a in the example shown) and the proximal-most anchor of the first section (anchor 30c in the example shown).

FIGS. 7D-E show a second section 123b of implant 122 being subsequently anchored along a second portion 10d of the path, by some more anchors 30 of the implant having been anchored to the tissue by tool 140. In the particular example shown, second section 123b comprises three more anchors 30, e.g., a fourth anchor 30d, a fifth anchor 30e, and a sixth anchor 30f. That is, in the particular example shown, anchor 30d is the distalmost anchor of second section 123b, and anchor 30f is the proximal-most anchor of second section 123b. Furthermore, in the particular example shown, sixth anchor 30f is the proximal-most anchor of implant 122 as a whole, and can thereby correspond generally to anchor 30z of implant 22. However, more or fewer anchors can be used. More generally, second section 123b can be defined as being between the distalmost anchor of the second section (anchor 30d in the example shown) and the proximal-most anchor of the second section (anchor 30d in the example shown).

Subsequently to anchoring first section 123a and prior to anchoring second section 123b, first portion 10c of the tissue (e.g., of the path along the tissue) is contracted by reducing a length of the first section, e.g., from a first length d1 (FIG. 7A) to a second length d1′ that is shorter than length d1 (FIG. 7B). Lengths d1 and d1′ can be measured along tether 26 between the distalmost anchor of first section 123a (anchor 30a in the example shown) and the proximal-most anchor of the first section (anchor 30c in the example shown). That is, they may represent the length of a first part 26a of the tether extending between the distalmost anchor of first section 123a (anchor 30a in the example shown) and the proximal-most anchor of the first section (anchor 30c in the example shown).

The contraction of first portion 10c of the tissue can be achieved by tensioning of tether 26, facilitated by an adjustment tool 160 that can provide a reference force (e.g., pressing against anchor 30c) while the tether is pulled proximally, e.g., as described for implant 22, mutatis mutandis. In some implementations, adjustment tool 160 is identical to adjustment tool 60, described hereinabove. In some implementations, delivery tool 140 is withdrawn from the subject prior to advancement of adjustment tool 160 to implant 122 (e.g., to anchor 30c). In some implementations, only driver 142 is withdrawn, and adjustment tool 160 is advanced through tube 150 to implant 122 (e.g., to anchor 30c).

While first portion 10c of the path remains contracted, the length of first section 123a is fixed, e.g., at length d1′ (FIG. 7B). This fixing can be achieved by locking a lock 24c to tether 26 at the proximal-most anchor of first section 123a-anchor 30c. For example, and as shown, lock 24c can be advanced (e.g., by adjustment tool 160) over and along tether 26, and/or can be locked to the tether just proximally from anchor 30c. Lock 24c can be as described for any other lock described herein.

Subsequently to anchoring second section 123b, and while the length of first section 123a remains fixed, second portion 10d of the tissue (e.g., of the path along the tissue) is contracted by reducing a length of the second section, e.g., from a first length d2 (FIG. 7E) to a second length d2′ that is shorter than length d2 (FIG. 7F). Lengths d2 and d2′ can be measured along tether 26 between the distalmost anchor of the second section (anchor 30d in the example shown) and the proximal-most anchor of the second section (anchor 30f in the example shown). That is, they may represent the length of a second part 26b of the tether extending between the distalmost anchor of second section 123b (anchor 30d in the example shown) and the proximal-most anchor of the second section (anchor 30f in the example shown).

While second portion 10d of the path remains contracted, length of second section 123b is fixed, e.g., at length d2′ (FIG. 7F). This fixing can be achieved using a lock 24f, e.g., as described hereinabove for lock 24z, mutatis mutandis. Similarly, excess tether 26 can be trimmed, e.g., by adjustment tool 60, as described hereinabove, mutatis mutandis.

In some implementations, lock 24c may merely inhibit re-increasing of the length of first section 123a, e.g., by obstructing sliding of tether 26 distally through the eyelet of anchor 30c. This may be sufficient for implementations in which tension in tether 26 is greater in section 123a than in section 123b. However, in some implementations, including some implementations in which tension may be greater in section 123b than in section 123a, an additional lock 24d is applied at the distalmost anchor of section 123b (anchor 30d in the example shown). For example, and as shown, lock 24d can be advanced (e.g., by adjustment tool 160) over and along tether 26, and/or can be locked to the tether just distally from where anchor 30d is to be positioned on the tether (FIG. 7C). This can be performed prior to the anchoring of anchor 30d (FIG. 7D). Lock 24d inhibits additional reduction of the length of first section 123a, by obstructing sliding of tether 26 proximally through the eyelet of anchor 30d. Thus, in some implementations, locks 24c and 24d can be used to fix the length of first section 123a prior to contraction of second section 123b.

In some implementations in which both lock 24c and lock 24d are used, tension in a part 26x of the tether, disposed between parts 26a and 26b (e.g., between locks 24c and 24d), may be different compared to that of both part 26a and part 26b. For example, part 26x may have little or no tension applied to it, e.g., it may actually be slack. Thus, a portion 10e of tissue 10 (e.g., of the path along the tissue), at which part 26x of tether 26 is disposed, may not be contracted despite contraction of portions 10c and 10d, which are disposed at either end of portion 10e, and which may delimit portion 10e. Thus, a non-contracting section 123x of implant 122 can be defined.

It is to be noted that, in accordance with some implementations, implant 122 and the techniques described for use therewith, advantageously facilitate contraction of first section 123a (and thereby the first portion of the path along the tissue) independently of contraction of second section 123b (and thereby the second portion of the path along the tissue)—as well as independently of a portion 10c of the path between the first and second sections. Thus, the operator may advantageously apply different degrees of contraction to different portions of the valve annulus, and/or may apply different magnitudes of tension to tether 26 in different sections of the implant, according to the requirements of the individual subject being treated.

It is to be noted that, although implant 122 is shown as having two contracting sections (section 123a and 123b) and a single non-contracting section 123x therebetween, it is to be understood that the scope of the present disclosure includes the implant having more contracting sections and/or more non-contracting sections. Furthermore, in some implementations, one or more non-contracting sections can be disposed at an end of the implant, e.g., not delimited by contracting sections. It is to be noted that, although implant 122 is showing as having three anchors per contracting section, the scope of the present disclosure includes the implant having a greater or smaller number of anchors per contracting section. It is to be noted that, although implant 122 is showing as having no anchors within non-contracting section (e.g., between locks 24c and 24d), the scope of the present disclosure includes the implant having one or more anchors within one or more of its non-contracting sections. More generally, it is to be understood that the scope of the present disclosure facilitates the operator deciding (during pre-procedure planning and/or during the implantation procedure itself) how to arrange the various contracting and non-contracting sections of the implant.

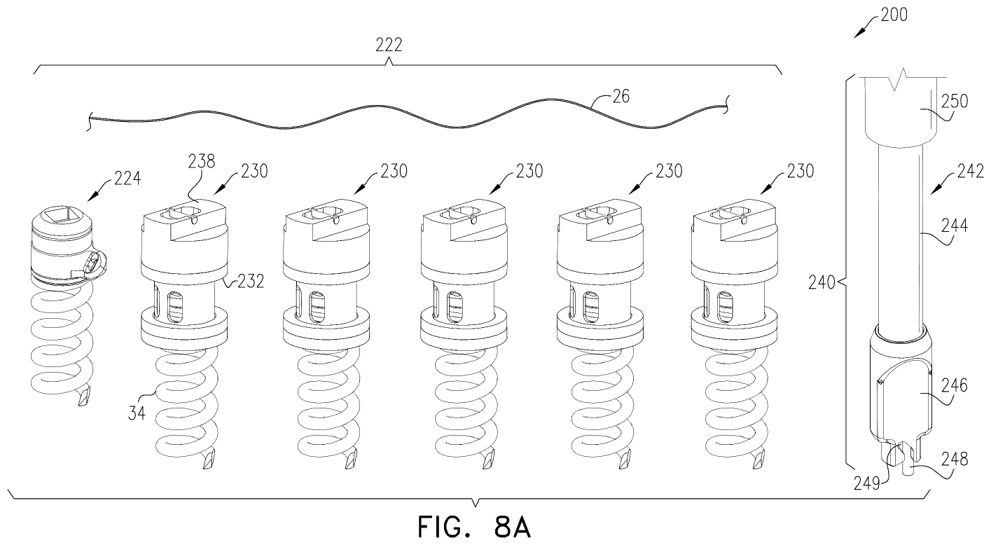

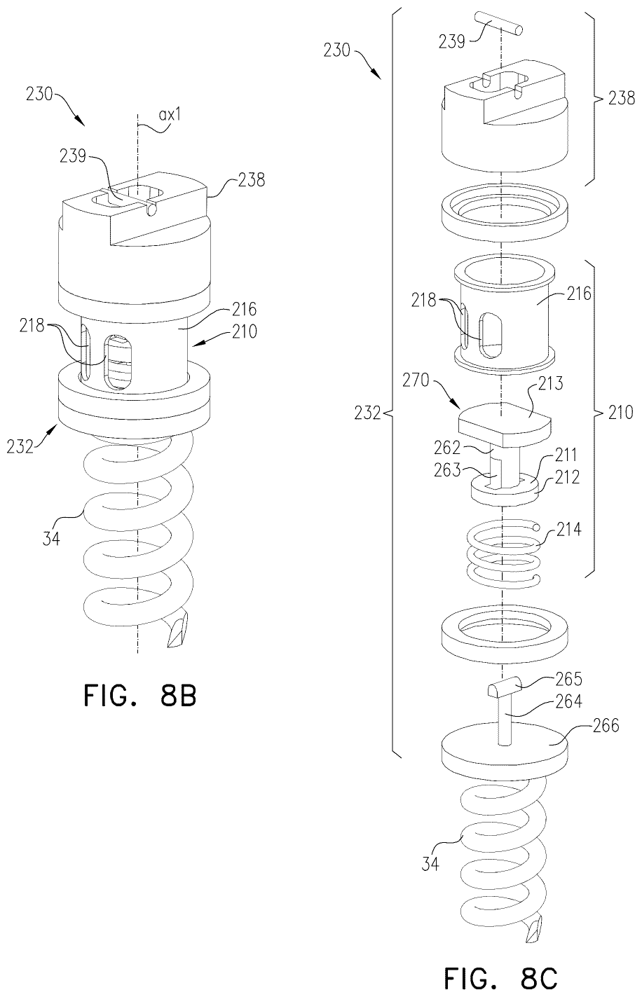

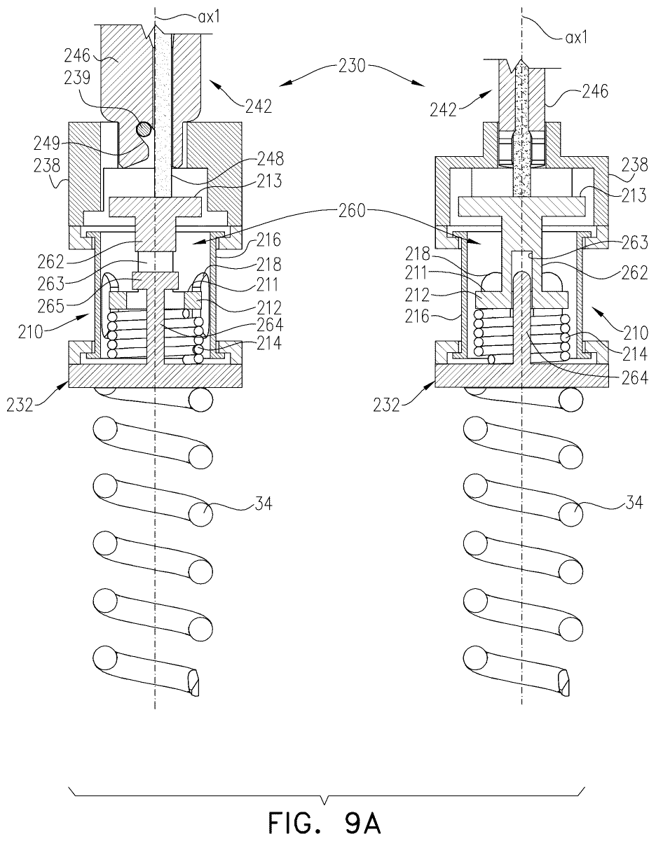

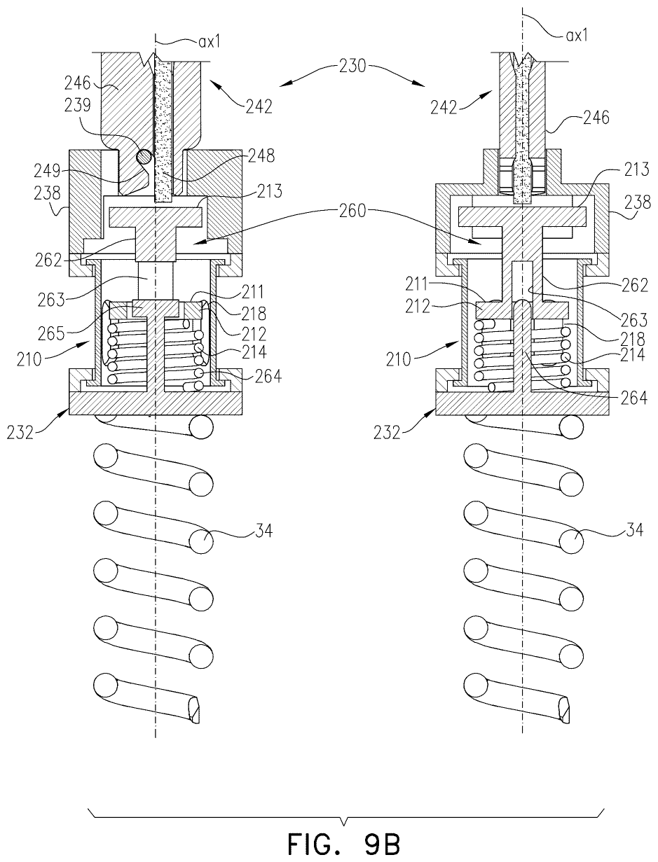

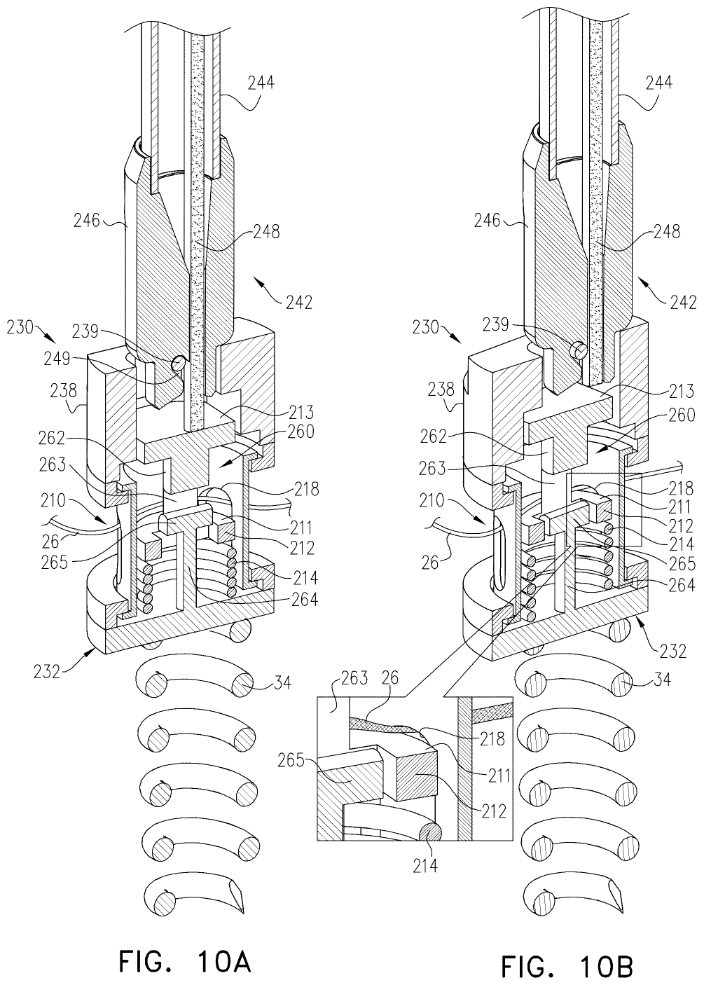

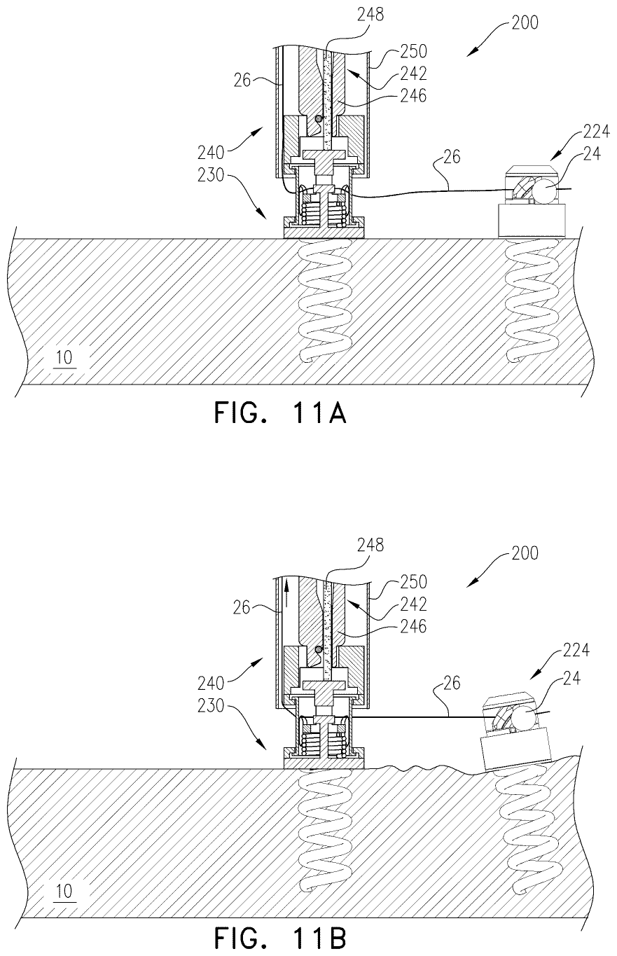

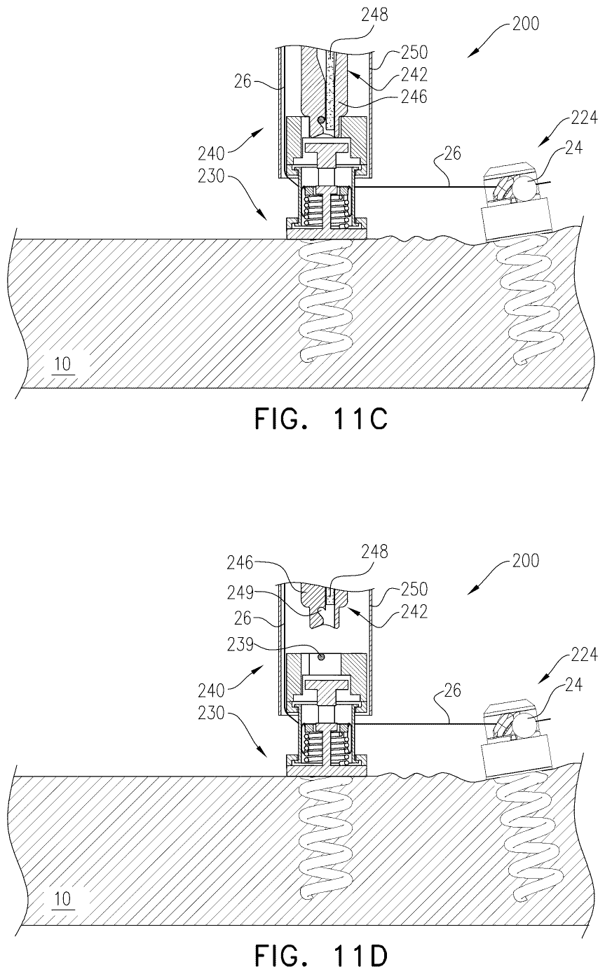

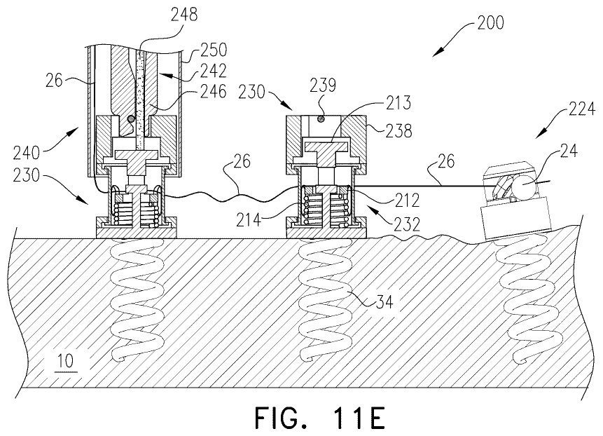

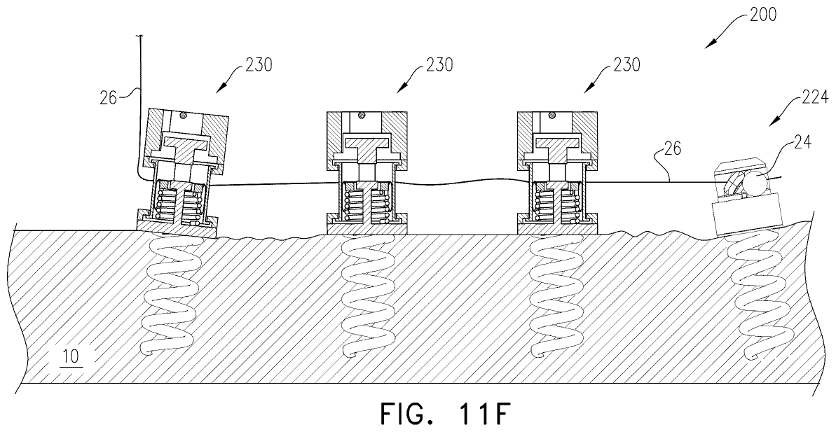

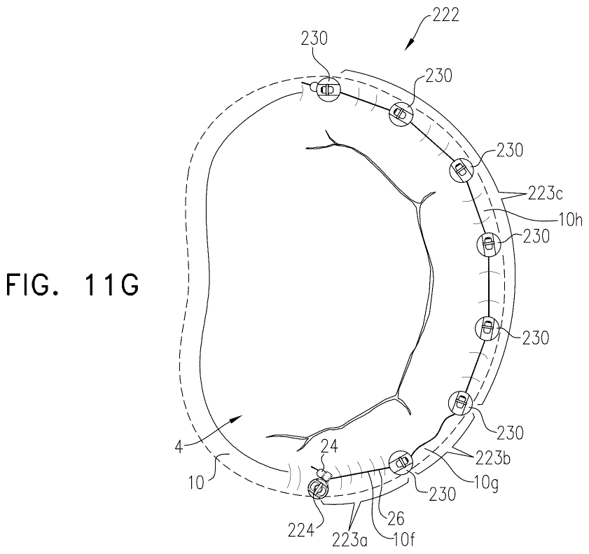

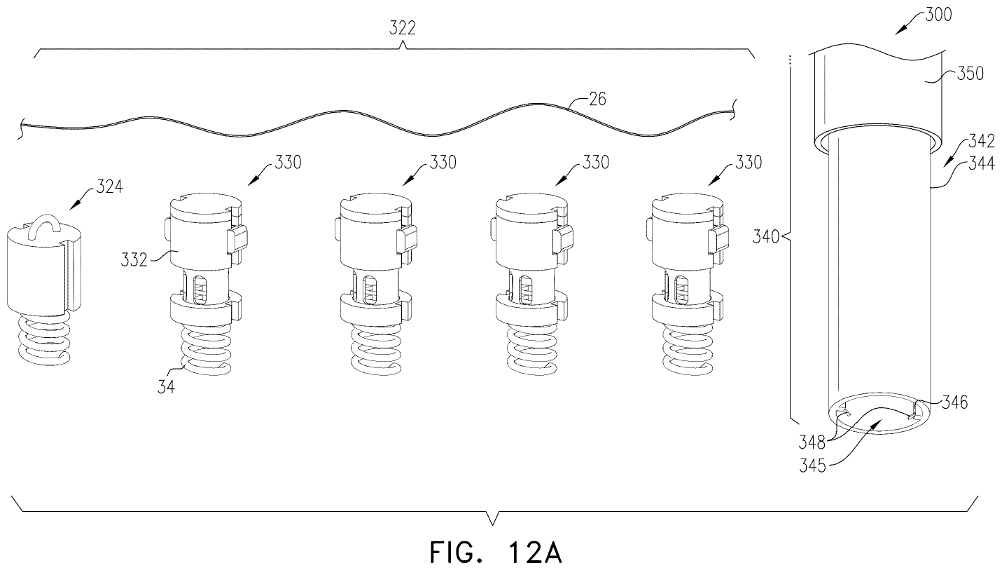

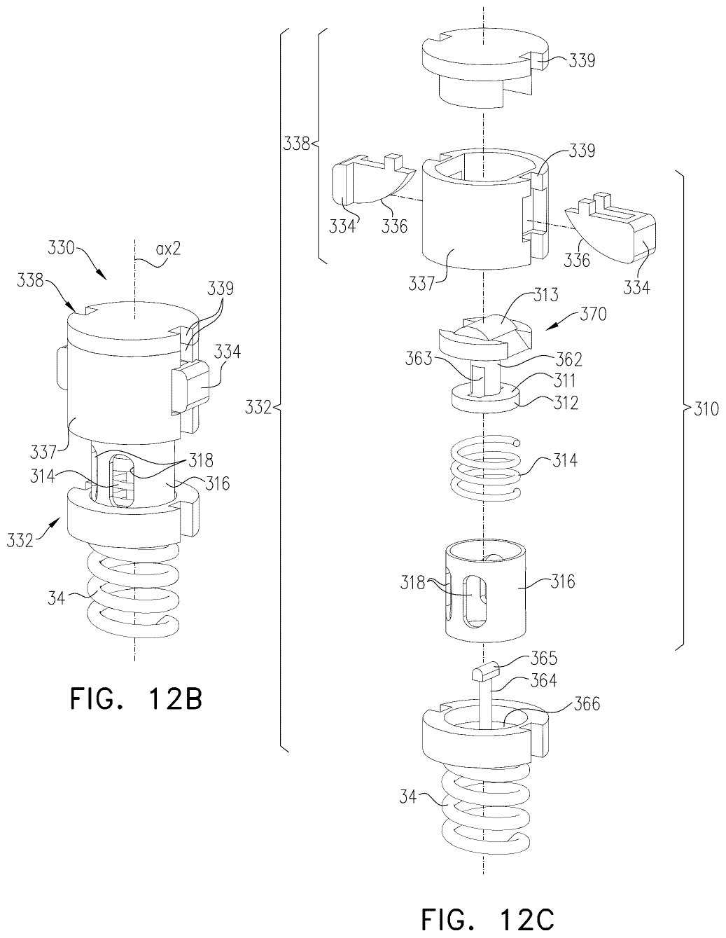

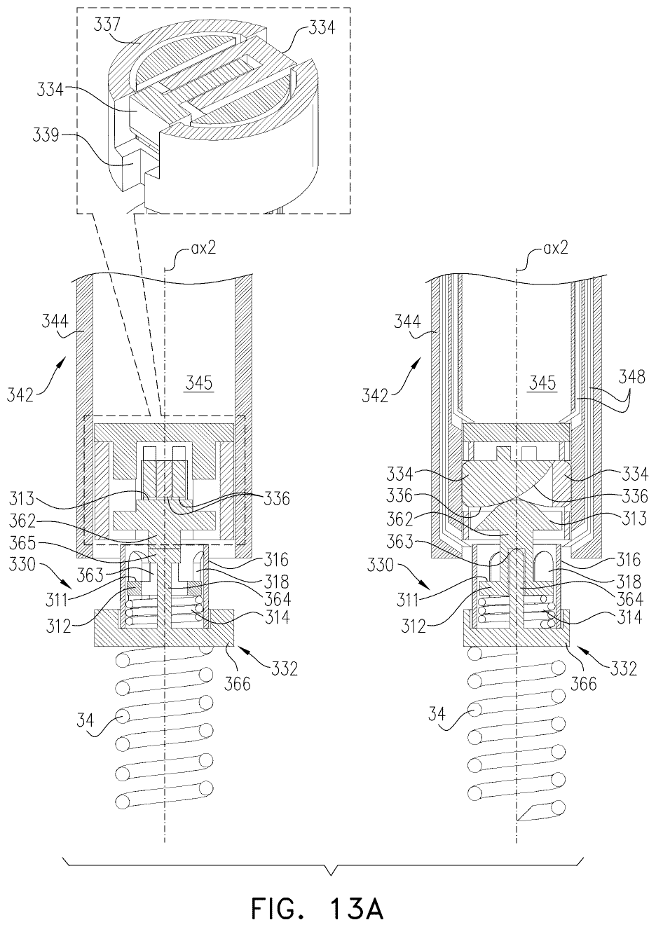

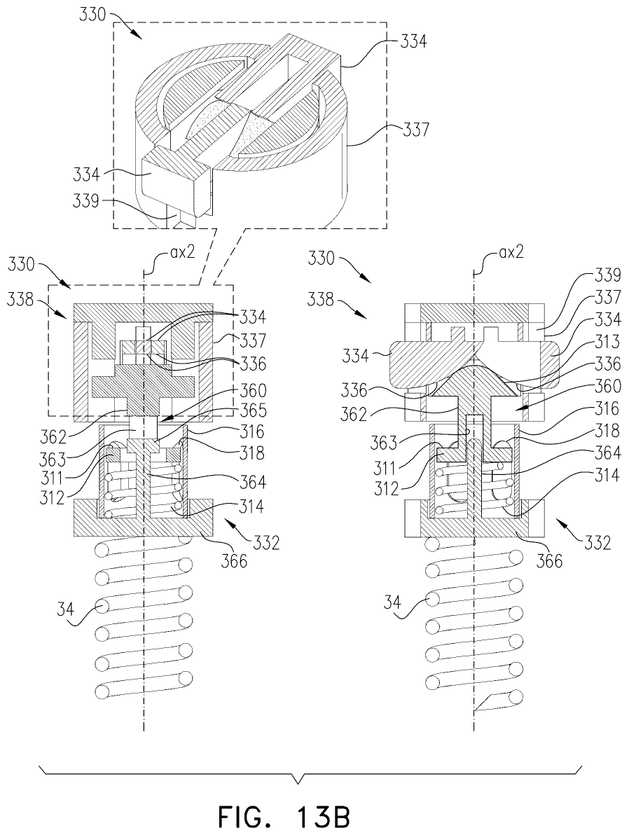

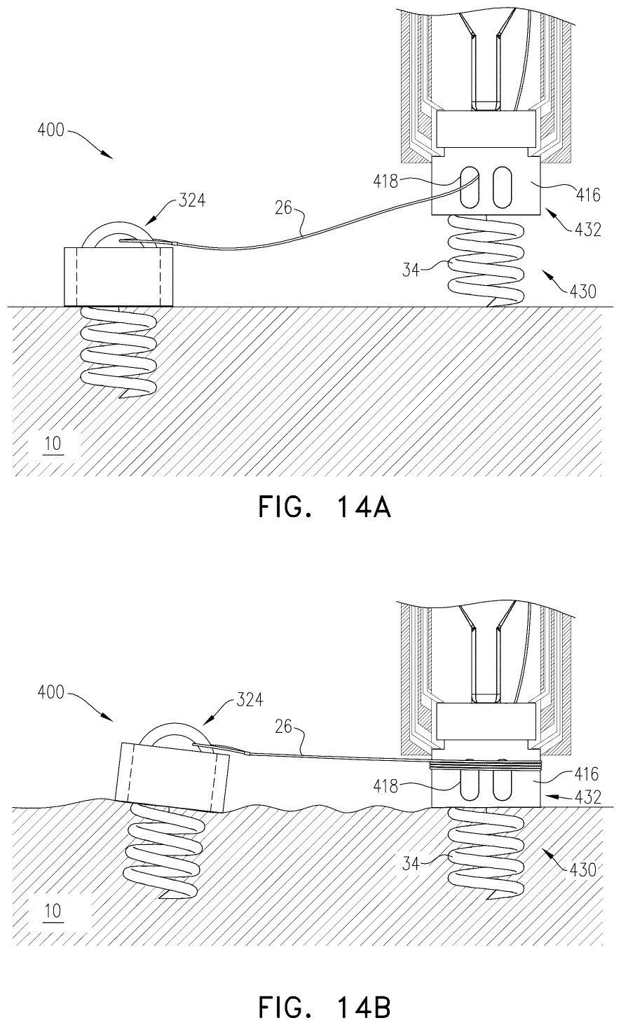

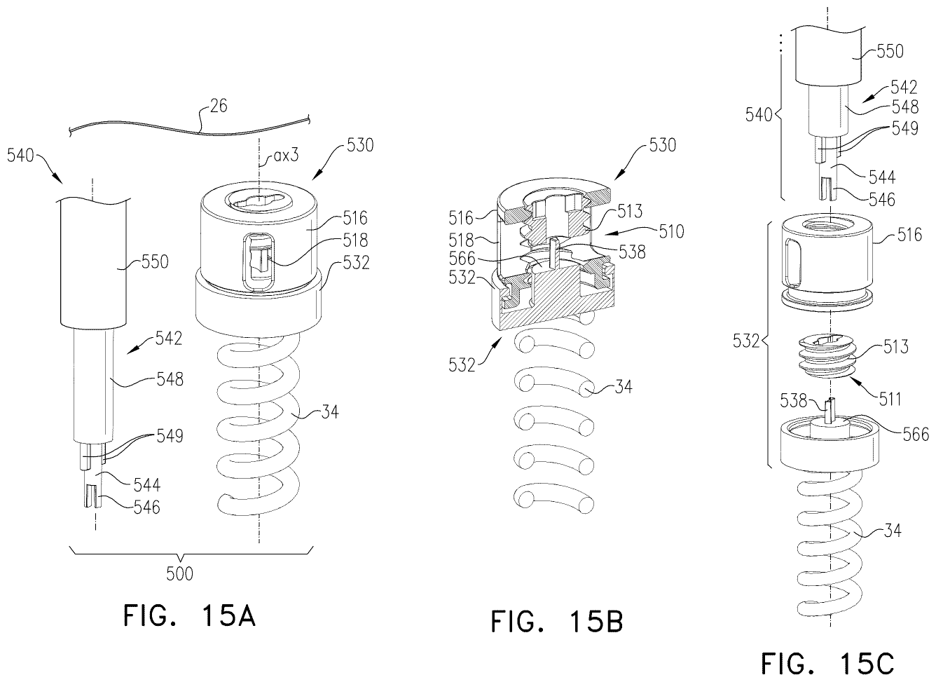

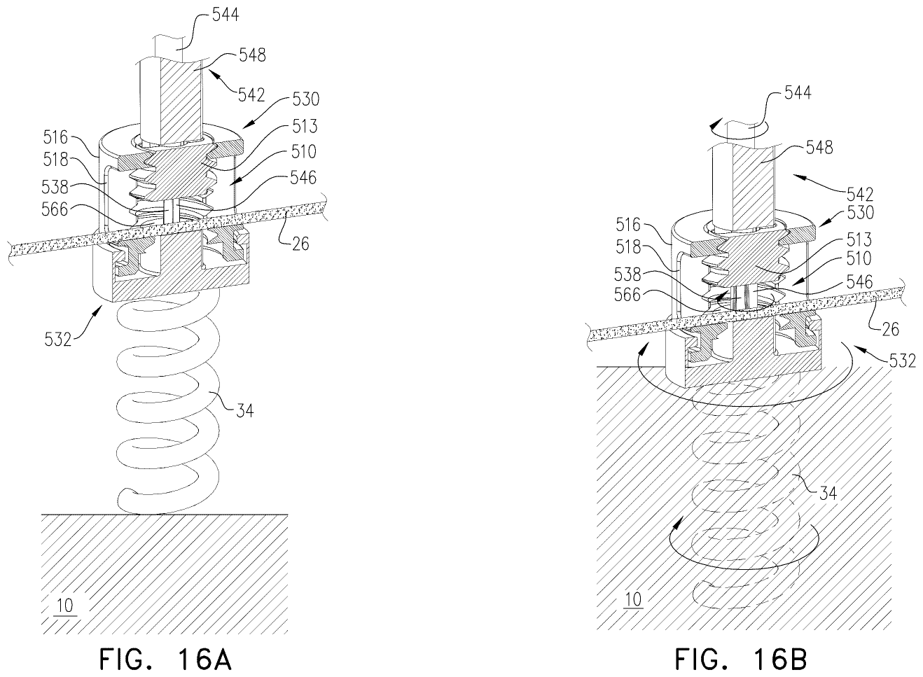

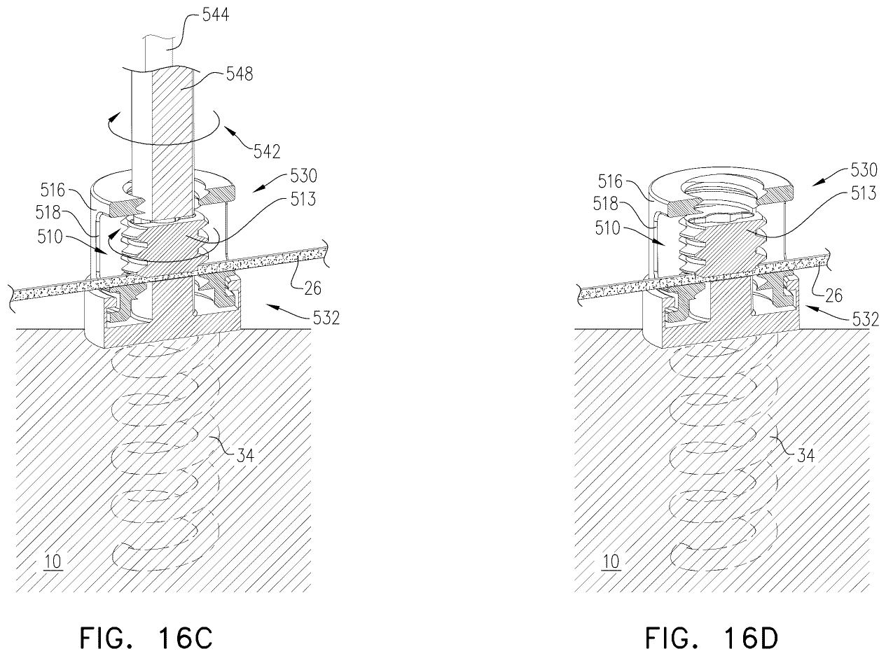

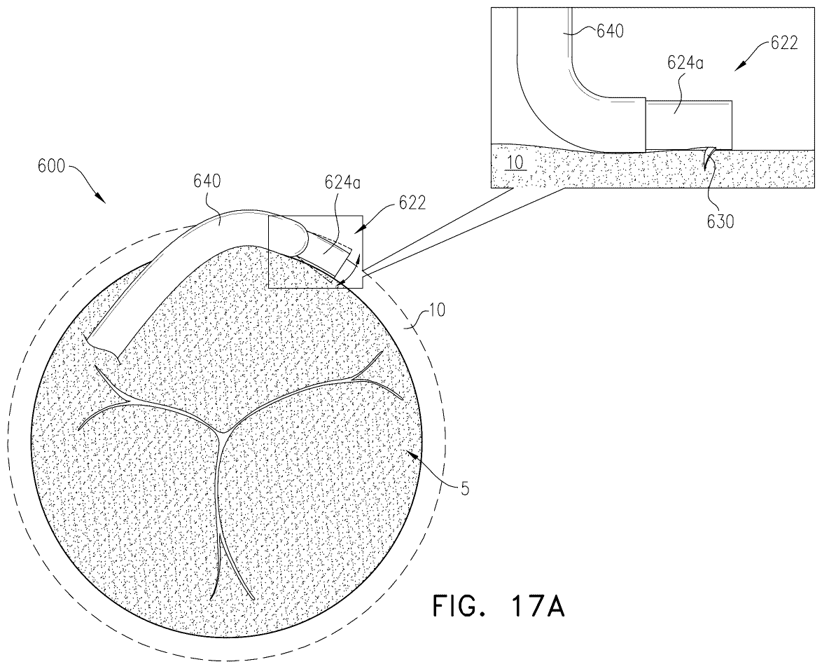

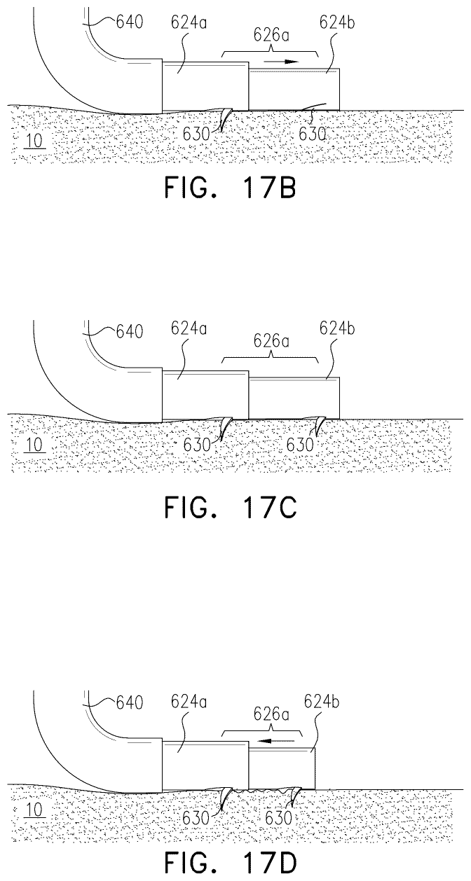

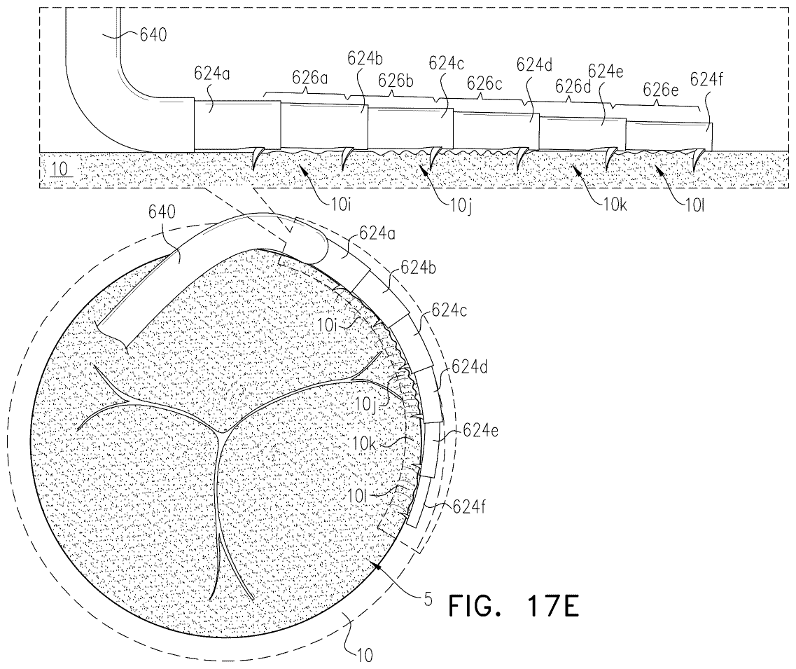

Reference is now made to FIGS. 8A-C, 9A-B, 10A-B, 11A-G, 12A-C, 13A-B, 14A-B, 15A-C, and 16A-D, which are schematic illustrations of systems and techniques for use with implants, in accordance with some implementations. In each case, the system comprises an implant and a delivery tool for implanting the implant, e.g., such that the implant defines a path along a tissue of the subject-such as along an annulus of a heart valve of the subject.

Therefore, with reference to FIGS. 8A-16D, there are provided, in accordance with some implementations, various systems for use at a tissue of a heart of a subject. Each such system comprises an implant, and can further comprise a delivery tool. The implant comprises an elongate member (e.g., tether 26, etc.), multiple anchors, and multiple locks. The delivery tool is configured to implant the implant at the tissue, such that the elongate member defines a path along the tissue, by (i) using one or more of the anchors to anchor a first section of the elongate member along a first portion of the path, and (ii) subsequently, using one or more of the anchors to anchor a second section of the elongate member along a second portion of the path. The delivery tool is further configured to contract the tissue by (i) subsequently to anchoring the first section and prior to anchoring the second section, contracting the first portion of the path by reducing a length of the first section, (ii) while the first portion of the path remains contracted, using at least a first lock of the multiple locks to fix the length of the first section, (iii) subsequently to anchoring the second section, and while the length of the first section remains fixed, contracting the second portion of the path by reducing a length of the second section, and (iv) while the second portion of the path remains contracted, using at least a second lock of the multiple locks to fix the length of the second section.

Similarly, in accordance with some implementations, a method comprising percutaneously implanting an elongate implant along the tissue such that the implant defines a path along the tissue by (i) anchoring a first section of the implant along a first portion of the path, and (ii) subsequently anchoring a second section of the implant along a second portion of the path. Subsequently to anchoring the first section and prior to anchoring the second section, (i) the first portion of the path is contracted by reducing a length of the first section, and (ii) while the first portion of the path remains contracted, the length of the first section is fixed. Subsequently to anchoring the second section, and while the length of the first section remains fixed, (i) the second portion of the path is contracted by reducing a length of the second section, and (ii) while the second portion of the path remains contracted, the length of the second section is fixed.

Similarly, in accordance with some implementations, a method comprising: (i) percutaneously implanting a first section of an implant along a first portion of the tissue by anchoring a first anchor of the implant to a first site of the tissue, and anchoring a second anchor of the implant to a second site of the tissue, the first portion of the tissue being disposed between the first site and the second site; (ii) subsequently, contracting the first portion of the tissue by reducing a length of the first section; (iii) while the first portion of the tissue remains contracted, fixing the length of the first section; (iv) subsequently, percutaneously implanting a second section of the implant along a second portion of the tissue by anchoring a third anchor of the implant to a third site of the tissue; (v) subsequently, while the length of the first section remains fixed, contracting the second portion of the tissue by reducing a length of the second section; and (vi) while the second portion of the tissue remains contracted, fixing the length of the second section.

Similarly, in accordance with some implementations, a method comprising (i) percutaneously anchoring a first anchor of an implant to a first site of the tissue; (ii) subsequently, percutaneously anchoring a second anchor of the implant to a second site of the tissue, a first portion of the tissue being disposed between the first site and the second site; (iii) subsequently, contracting the first portion of the tissue by drawing together the first anchor and the second anchor; (iv) while the first portion of the tissue remains contracted, fixing a distance between the first anchor and the second anchor; (v) subsequently, percutaneously changing a distance between a third anchor of the implant and the second anchor by sliding the third anchor with respect to the second anchor; (vi) subsequently, anchoring the third anchor to a third site of the tissue, a second portion of the tissue being disposed between the second site and the third site; (vii) subsequently, while the distance between the first anchor and the second anchor remains fixed, contracting the second portion of the tissue by drawing together the second anchor and the third anchor; and (viii) while the second portion of the tissue remains contracted, fixing a distance between the second anchor and the third anchor.

In some implementations, the above is provided or facilitated by the system (e.g., the implant of the system) including a series of anchors slidably coupled to a tether. In each of systems 200 (FIGS. 8A-11G), 300 (FIGS. 12A-13B), 400 (FIGS. 14A-B), and 500 (FIGS. 15A-16D), the implant comprises a tether and a series of anchors, each anchor comprising a tissue-engaging element (e.g., one or more of a helix, screw, point, hook, barb, dart, arm, staple, sharpened portion, clip, pledget, etc.) and each anchor can also optionally also include a head. In some implementations, the anchor (e.g., a head thereof, etc.) is slidably coupled (or couplable) to the tether, and comprises a lock configured, upon locking thereof, to lock the head to the tether. These systems are configured to facilitate techniques by which sections of the implant are adjustable independently of other sections, e.g., such that portions of the path along which the implant is implanted are contractable independently of other portions of the path. In some implementations, these techniques can be considered to be broadly similar to those described with reference to FIGS. 7A-F, mutatis mutandis.

FIGS. 8A-11G are schematic illustrations of a system 200 and techniques for use therewith, in accordance with some implementations. FIG. 8A shows an overview of system 200. System 200 comprises an implant 222 that comprises a tether and a series of anchors 230. In the example shown, the tether of implant 222 is tether 26 (described hereinabove), but implant 222 can alternatively or additionally comprise another tether, mutatis mutandis. System 200 is shown as having five anchors 230, but it is to be understood that this is illustrative, and the system can have more or fewer anchors 230. System 200 can further comprise a delivery tool 240 for percutaneous (e.g., transluminal, such as transfemoral) implantation of the implant, e.g., via anchoring of anchors 230.

Although FIG. 8A shows the tether of implant 222 separate from the anchors of the implant, the implant can be provided with the anchors already connected to the tether, e.g., as described in International Patent Application PCT/IB2021/058665 to Halabi et al., which published as WO 2022/064401, and/or International Patent Application PCT/IB2022/051099 to Shafigh et al., mutatis mutandis. In some implementations, implant 222 can be provided with one or more of the anchors separate from the tether, and configured to be connected to the tether.

FIG. 8B shows one of anchors 230, and FIG. 8C shows an exploded view of the anchor. Each anchor 230 comprises a tissue-engaging element 34 (e.g., one or more of a helix, screw, point, hook, barb, dart, arm, staple, sharpened portion, clip, pledget, etc.), and can also comprise a head 232 coupled to the tissue-engaging element (e.g., to a proximal end of the tissue-engaging element). In some implementations, the anchor (e.g., head 232, etc.) is slidably coupled, or is configured to be slidably coupled, to tether 26, e.g., as described in more detail hereinbelow. Although in the example shown the tissue-engaging element of anchor 230 is tissue-engaging element 34 (described hereinabove), anchor 230 can alternatively or additionally comprise another tissue-engaging element, mutatis mutandis. In some implementations, head 232 comprises a lock 210 that is configured, upon locking thereof, to lock the head to tether 26.

In some implementations, implant 222 can comprise a leading anchor 224 whose tissue-engaging element can be similar to that of anchors 230, but whose head may not have the locking functionality of head 232 (e.g., may not comprise lock 210). Rather, leading anchor 224 can be fixedly (rather than slidably) coupled to tether 26 directly, or can be inhibited from sliding along and/or off of the tether by one or more separate locks or stoppers, such as lock 24, described hereinabove. In some implementations, leading anchor 224 can be as described hereinabove for anchor 30, mutatis mutandis. In some implementations, leading anchor 224 can be as described hereinbelow for leading anchor 324, mutatis mutandis.

Delivery tool 240 is configured to transluminally advance anchors 230 (and thereby typically implant 222 in general) to the heart of the subject. Delivery tool 240 can comprise a driver 242. Delivery tool 240 can also comprise a flexible tube (e.g., a catheter) 250 that is transluminally advanceable to the heart, and through which driver 242 is extendable. Delivery tool 240 can be configured to, for each anchor 230 sequentially, (i) anchor the anchor to the tissue, such that a section of tether 26 extends to the anchor from a preceding one of the anchors (e.g., the anchor to have been most recently anchored), subsequently adjust tension on the section of the tether, and subsequently lock 210 of the anchor. FIGS. 11A-G illustrate this by showing some of the steps in the implantation of implant 222, in accordance with some implementations.

In some implementations, delivery tool 240 is also configured to anchor leading anchor 224. For example, a driver interface of the head of leading anchor 224 can be similar to that of anchors 230. In some implementations, a separate delivery tool is provided and used to anchor leading anchor 224.

FIGS. 9A-B show anchor 230 in longitudinal cross-section whereby, for each of these figures, the cross-section of the left frame is orthogonal to the cross-section of the right frame. That is, for each of FIGS. 9A-B, a central longitudinal axis ax1 of anchor 230 (e.g., defined by tissue-engaging element 34) lies on the respective plane of both cross-sections, with the plane of the cross-section of one frame rotated 90 degrees about axis ax1 with respect to the plane of the cross-section of the other frame.

FIGS. 10A-B also show anchor 230 in longitudinal cross-section, although in perspective view. Except for this different viewing angle, the cross-section of FIG. 10A is the same as that of the left frame of FIG. 9A, and the cross-section of FIG. 10B is the same as that of the left frame of FIG. 9B. However, FIGS. 10A-B also show tether 26 threaded laterally through head 232.

FIGS. 9A and 10A show anchor 230 in its unlocked state, and FIGS. 9B and 10B show the anchor in its locked state.

In some implementations, and as shown, lock 210 defines a press surface 211, and is configured to lock head 232 to tether 26 by pressing the press surface against the tether. As shown, lock 210 can comprise a press plate 212 that defines the press surface.

Lock 210 can comprise a spring 214 configured to lock head 232 to tether 26 by pressing press surface 211 against the tether, e.g., biasing lock 210 to lock. In some implementations, spring 214 is configured to press press surface 211 against tether 26 by moving the press surface axially with respect to tissue-engaging element 34. In the example shown, press surface 211 is a proximal surface of press plate 212 (e.g., a surface of the press plate that faces away from tissue-engaging element 34), and spring 214 is configured to push press surface 211 proximally (e.g., away from the tissue-engaging element). For example, spring 214 can be a compression spring disposed between press plate 212 and tissue-engaging element 34. In the particular example shown, spring 214 is disposed between press plate 212 and a base plate 266 of head 232. Base plate 266 can be fixedly attached to tissue-engaging element 34.

In some implementations, and as shown, head 232 (e.g., lock 210 thereof) can comprise or define a circumferential wall 216 that circumscribes (e.g., is coaxial with) central longitudinal axis ax1, and that has one or more apertures 218 defined therein. In some implementations, head 232 is slidably coupled to tether 26 by the tether extending transversely through the head via apertures 218. In some implementations, press surface 211 can be disposed medially from circumferential wall 216 (e.g., the circumferential wall circumscribes press plate 212).

In some implementations in which head 232 comprises wall 216 with apertures 218, spring 214 can be configured to move press surface 211 across at least one of apertures 218. This can lock head 232 to tether 26 by pinching the tether between the press surface 211 and a rim of aperture 218, e.g., as indicated by the arrows in the inset of FIG. 10B.

Lock 210 can be unlocked, or can be inhibited from locking, by a force applied by driver 242. For example, a control rod 248 of driver 242 can apply the force (e.g., push distally) against a surface (e.g., a proximal-facing surface) 213 defined by or linked to press plate 212, thereby straining spring 214 (e.g., compressing the spring against base plate 266) or inhibiting the spring from relaxing (FIGS. 9A and 10A). Thus, surface 213 can serve as a push-button. Retraction of control rod 248 allows spring 214 to relax (e.g., to decompress), transitioning lock 210 toward its locked state (FIGS. 9B and 10B).

In some implementations, such as in some implementations in which tissue-engaging element 34 is a helical/screw-in tissue-engaging element, the tissue-engaging element can be rotatable independently of circumferential wall 216. This enables tissue-engaging element 34 to be screwed into the tissue without wrapping tether 26 around the anchor, e.g., with the tether holding wall 216 stationary while the tissue-engaging element rotates. The driving (e.g., screwing) of tissue-engaging element 34 into the tissue can be performed by driver 242. Driver 242 can comprise a drive head 246, and a shaft 244 that extends from an extracorporeal portion (e.g., a control handle) to the drive head. Drive head 246 is reversibly engageable with a driver interface 238 of head 232 of anchor 230. FIG. 11A shows an anchor 230 having been screwed into tissue 10 while tether 26 extends from a previously-anchored anchor (in this case, leading anchor 224), laterally through head 232 of the anchor 230 that is being anchored, and proximally away, without the tether becoming wrapped around head 232.

In some implementations, and as shown, anchor 230 (e.g., head 232 thereof) comprises a torque assembly 260 via which torque is transferred from interface 238 to tissue-engaging element 34. Torque assembly 260 can be disposed medially from circumferential wall 216, and can comprise one or more torque shafts that extend between interface 238 and tissue-engaging element 34. These torque shaft(s) can lie on central longitudinal axis ax1. In the example shown, torque assembly 260 comprises at least a first torque shaft 262 and a second torque shaft 264, which are operatively coupled to each other to transfer torque, e.g., due to their respective shapes rotationally locking them to each other. For example, and as shown, a noncircular and/or protruding part 265 of torque shaft 264 can cooperate with a noncircular and/or recessed part 263 of torque shaft 262 to define a keyed joint.

Thus, in some implementations, head 232 comprises a casing that has a proximal part (e.g., including interface 238), and a base part (e.g., including base plate 266) via which the head is coupled to tissue-engaging element 34. The proximal and distal parts of the head are axially separated at a mid-section at which circumferential wall 216 is disposed. Although the proximal part, the base part, and the tissue-engaging element are rotationally (and typically axially) locked with respect to each other, they are (e.g., collectively) rotatably coupled to circumferential wall 216. Lock 210 and/or circumferential wall 216 can axially traverse the mid-section.

In some implementations, and as shown, some components of head 232 can be common to both torque assembly 260 and lock 210. For example, and as shown, a unitary component 270 can be shaped to define surface 213, shaft 262, and press plate 212 (see FIG. 8C). Component 270 can be shaped and/or keyed to be rotationally locked to interface 238. For example, and as shown, surface 213 can be noncircular. In some implementations, shaft 262 thereby also serves to transfer the unlocking force that is applied to surface 213 by control rod 248, e.g., the shaft pushing press plate 212 against spring 214, thereby compressing the spring between the press plate and base plate 266. Thus, in some implementations, component 270 can be considered to be a piston. In order to provide this functionality, the operative coupling between shafts 262 and 264 can allow shaft 262 to slide axially with respect to shaft 264. For example, and as shown, part 263 can be a groove or slit that runs substantially parallel with axis ax1, dimensioned to allow part 265 to slide axially therealong.

In some implementations, and as shown (e.g., in FIGS. 10A-B), tether 26 extends laterally through head 232 by passing (i) into an aperture 218, (ii) across press plate 212 and/or past (e.g., at least partway around) torque assembly 260 (e.g., torque shaft 262 and/or torque shaft 264), and (ii) out of another aperture 218.

As described hereinabove, drive head 246 of driver 242 is reversibly engageable with interface 238. This engagement can be controlled via control rod 248. For example, and as shown, while control rod 248 is in a retracted position a chamber (or recess) 249 defined by drive head 246 can receive and release a crossbar 239 (and/or another component) of interface 238; and advancement of the control rod distally from the retracted position while the crossbar is disposed in the chamber can lock the crossbar to the drive head. Therefore, in some implementations, control rod 248 controls both (i) engagement between driver 242 and anchor 230, and (ii) lock 210. For example, in some implementations, while driver 242 is engaged with anchor 30, (i) control rod 248 being in an advanced position maintains the engagement and maintains lock 210 unlocked (see, for example, FIGS. 9A, 10A, and 11A-B); (ii) retraction of the control rod into an intermediate position maintains the engagement but allows the lock to lock (see, for example, FIGS. 9B, 10B, and 11C); and (iii) further retraction of the control rod into a retracted position causes or allows disengagement of the driver from the anchor while the lock remains locked (see, for example, FIG. 11D).

FIG. 11A shows leading anchor 224 and one anchor 230 having been anchored to tissue 10, with tether 26 extending from the leading anchor, through the anchor 230 (as described hereinabove), and proximally away from the tissue (e.g., out of the heart, and possibly out of the subject entirely), e.g., via delivery tool 240 (e.g., tube 250 thereof). Subsequently, tether 26 is tensioned to a desired degree of tension (FIG. 11B), e.g., guided by one or more imaging techniques (such as fluoroscopy and/or ultrasonography) to determine the effect of the tensioning on the structure and/or function of the tissue and/or surrounding structures, such as the effect on regurgitation through a heart valve. At this point, repeated increasing and decreasing of tension is possible in order to determine a desired degree of tension. Once it has been determined that the desired degree of tension has been achieved, lock 210 of anchor 230 is locked to tether 26 (FIG. 11C), e.g., by retracting rod 248, as described hereinabove-thereby locking the applied tension into the section of the tether (e.g., of implant 222) between leading anchor 224 and the first anchor 230. Subsequently, tool 240 (e.g., driver 242 thereof) is disengaged from anchor 230, e.g., by further retraction of rod 248 (FIG. 11D). Driver 242 (and optionally tool 240 in its entirety) can then be retracted from the subject, and then used to advance another anchor 230 (e.g., by sliding it along tether 26) and anchor it to the tissue (FIG. 11E). Because lock 210 of the first anchor 230 has been locked to tether 26, tension in sections of the tether proximal from the first anchor 230 can be different (e.g., smaller or greater) than that in the section of the tether between leading anchor 224 and the first anchor 230. FIG. 11E shows less (e.g., substantially no) tension in the tether proximal from the first anchor 230. FIG. 11F shows this lower degree of tension having been fixed (i.e., “locked in”) in the section of tether 26 between the first anchor 230 and the second anchor 230, by locking the second anchor 230 to the tether. In this manner, implant 222 can be progressively anchored and tensioned until implantation and adjustment is complete. FIG. 11G shows an example in which implantation and adjustment of implant 222 has been completed, with the implant defining a path around the annulus of a mitral valve 4, e.g., around the posterior annulus, roughly from commissure to commissure.

Thus, in the particular example illustrated, once implant 222 has been implanted such that it defines a path along tissue 10, and has been differentially contracted as described hereinabove, (i) a portion 10f of the path, along which a first section 223a of the implant has been secured (e.g., anchored), has been contracted by a first amount of contraction; (ii) a portion 10g of the path, along which a second section 223b of the implant has been secured, has substantially not been contracted; and (iii) a portion 10h of the path, along which a third section 223c of the implant has been secured, has been contracted by a second amount of contraction that is less than the first amount of contraction. In the example shown, section 223a extends between leading anchor 224 and the first of anchors 230 to have been anchored, section 223b extends between the first and second of anchors 230 to have been anchored, and section 223c extends between the second of anchors 230 to have been anchored and the final anchor of the implant.

It is to be noted that, although the final anchor of implant 222 to be anchored is shown as an anchor 230, which itself comprises lock 210, in some implementations an additional/discrete lock (e.g., another lock 24) can nonetheless be added and locked to tether 26 adjacent the final anchor, for the sake of additional locking strength, e.g., as shown in FIG. 11G. Furthermore, in some implementations, the final anchor of implant 222 to be anchored can be an anchor other than an anchor 230, e.g., can be an anchor that does not itself comprise a lock, in which case an additional/discrete lock can be necessary.

FIGS. 12A-13B are schematic illustrations of a system 300, in accordance with some implementations. FIG. 12A shows an overview of system 300. System 300 comprises an implant 322 that comprises a tether and a series of anchors 330. In the example shown, the tether of implant 322 is tether 26 (described hereinabove), but implant 322 can alternatively or additionally comprise another tether, mutatis mutandis. System 300 is shown as having four anchors 330, but it is to be understood that this is illustrative, and the system can have more or fewer anchors 330. System 300 can further comprise a delivery tool 340 for percutaneous (e.g., transluminal, such as transfemoral) implantation of the implant, e.g., via anchoring of anchors 330.

Although FIG. 12A shows the tether of implant 322 separate from the anchors of the implant, the implant can be provided with the anchors already connected to the tether, e.g., as described in International Patent Application PCT/IB2021/058665 to Halabi et al., which published as WO 2022/064401, and/or International Patent Application PCT/IB2022/051099 to Shafigh et al. In some implementations, implant 322 can be provided with one or more of the anchors separate from the tether and configured to be connected to the tether.

FIG. 12B shows one of anchors 330, and FIG. 12C shows an exploded view of the anchor. Each anchor 330 comprises a tissue-engaging element 34 and can optionally also include a head 332 coupled to the tissue-engaging element (e.g., to a proximal end of the tissue-engaging element). In some implementations, the anchor (e.g., head 332, etc.) is slidably coupled, or is configured to be slidably coupled, to tether 26, e.g., in a similar way to head 232 of anchor 230, mutatis mutandis. Although in the example shown the tissue-engaging element of anchor 330 is tissue-engaging element 34 (described hereinabove), anchor 330 can alternatively or additionally comprise another tissue-engaging element, mutatis mutandis. In some implementations, head 332 comprises a lock 310 that is configured, upon locking thereof, to lock the head to tether 26.

In some implementations, system 300 can be considered to be a variation of system 200, and can be used similarly, mutatis mutandis, the primary differences being the mechanism of lock 310 compared to that of lock 210, and the manner in which anchors 330 are engaged by their anchor driver.

In some implementations, implant 322 can comprise a leading anchor 324 whose tissue-engaging element can be similar to that of anchors 330, but whose head may not have the locking functionality of head 332 (e.g., may not comprise lock 310). Rather, leading anchor 324 can be fixedly (rather than slidably) coupled to tether 26 directly (e.g., as shown), or can be inhibited from sliding along and/or off of the tether by one or more separate locks or stoppers, such as lock 24 (e.g., as shown for leading anchor 224 of implant 222). In some implementations, leading anchor 324 can be as described hereinabove for anchor 30, mutatis mutandis.

Delivery tool 340 is configured to transluminally advance anchors 330 (and thereby typically implant 322 in general) to the heart of the subject. Delivery tool 340 can comprise a driver 342. Delivery tool 340 can also comprise a flexible tube (e.g., a catheter) 350 that is transluminally advanceable to the heart, and through which driver 342 is extendable. Tube 350 can be similar or identical to tube 250 described hereinabove. Delivery tool 340 can be configured to, for each anchor 330 sequentially, (i) anchor the anchor to the tissue, such that a section of tether 26 extends to the anchor from a preceding one of the anchors (i.e., the anchor to have been most recently anchored), subsequently adjust tension on the section of the tether, and subsequently lock 310 of the anchor. The implantation of implant 322 can be broadly similar to that of implant 222, and/or similar to as shown in FIGS. 11A-G, mutatis mutandis.

In some implementations, delivery tool 340 is also configured to anchor leading anchor 324. For example, a driver interface of the head of leading anchor 324 can be similar to that of anchors 330. In some implementations, a separate delivery tool is provided and used to anchor leading anchor 324.

FIGS. 13A-B show anchor 330 in longitudinal cross-section whereby, for each of these figures, the cross-section of the left frame is orthogonal to the cross-section of the right frame. That is, for each of FIGS. 13A-B, a central longitudinal axis ax2 of anchor 330 (e.g., defined by tissue-engaging element 34) lies on the respective plane of both cross-sections, with the plane of the cross-section of one frame rotated 90 degrees about axis ax2 with respect to the plane of the cross-section of the other frame. FIGS. 13A-B are therefore similar to FIGS. 9A-B, but showing anchor 330 instead of anchor 230.

FIG. 9A shows anchor 330 in its unlocked state, and FIG. 9B shows the anchor in its locked state.

In some implementations, and as shown, lock 310 defines a press surface 311, and is configured to lock head 332 to tether 26 by pressing the press surface against the tether. As shown, lock 310 can comprise a press plate 312 that defines the press surface.

Lock 310 can comprise a spring 314 configured to lock head 332 to tether 26 by pressing press surface 311 against the tether, e.g., biasing lock 310 to lock. In some implementations, spring 314 is configured to press press surface 311 against tether 26 by moving the press surface axially with respect to tissue-engaging element 34. In the example shown, press surface 311 is a proximal surface of press plate 312 (e.g., a surface of the press plate that faces away from tissue-engaging element 34), and spring 314 is configured to push press surface 311 proximally (e.g., away from the tissue-engaging element). For example, spring 314 can be a compression spring disposed between press plate 312 and tissue-engaging element 34. In the particular example shown, spring 314 is disposed between press plate 312 and a base plate 366 of head 332. Base plate 366 can be fixedly attached to tissue-engaging element 34.

In some implementations, and as shown, head 332 (e.g., lock 310 thereof) can comprise or define a circumferential wall 316 that circumscribes (e.g., is coaxial with) central longitudinal axis ax2, and that has one or more apertures 318 defined therein. In some implementations, head 332 is slidably coupled to tether 26 by the tether extending transversely through the head via apertures 318. In some implementations, press surface 311 can be disposed medially from circumferential wall 316 (e.g., the circumferential wall circumscribes press plate 312).

In some implementations in which head 332 comprises wall 316 with apertures 318, spring 314 can be configured to move press surface 311 across at least one of apertures 318. This can lock head 332 to tether 26 by pinching the tether between the press surface 311 and a rim of aperture 318.

Lock 310 can be unlocked, or can be inhibited from locking, by a force applied by driver 342. For example, driver 242 can indirectly push (e.g., distally) a surface (e.g., a proximal-facing surface) 313 defined by or linked to press plate 312, thereby straining spring 314 (e.g., compressing the spring against base plate 366) or inhibiting the spring from relaxing (FIG. 9A). This indirect pushing can be achieved by driver 242 pushing one or more lateral push-buttons 334 that are components of head 332. In the example shown, push-buttons 334 are lateral push-buttons, e.g., mounted laterally on head 332, and optionally protruding laterally from a casing 337 of the head. Thus, lock 310 can be configured such that when spring 314 moves press surface 311 axially with respect to (e.g., away from) tissue-engaging element 34, that movement projects push-buttons 334 laterally from casing 337. Pressing push-buttons 334 medially unlocks lock 310 by moving press surface 311 axially, e.g., with respect to the casing and/or with respect to tissue-engaging element 34.

In the example shown, this functionality is provided by (i) each push-button 334 defining (or being linked to) a respective bearing surface 336, and (ii) surface 313 (which can also be considered to be a bearing surface) being oblique with respect to surface(s) 336 such that, for each push-button 334, pressing the push-button medially moves press surface 311 axially by sliding surface 336 over surface 313. Surface 336 and/or surface 313 can be oblique with respect to axis ax2. As shown, each push-button 334 can define (or can be attached to) a wedge that defines surface 336, e.g., the push-button can be wedge-shaped.

In some implementations, and as shown, anchor 330 can comprise two push-buttons 334, e.g., opposite each other. In some implementations, and as shown, parts of the two push-buttons slide past each other when the push-buttons are pushed medially. As shown, the two push-buttons can be shaped to slide into each other when pushed medially.

In some implementations, and as shown, surface 313 is curved (e.g., convex). For example, and as shown, surface 313 can be shaped as arch or a dome. In some implementations, and as shown, surface 336 is curved (e.g., convex).

In some implementations, and as shown, driver 242 can comprise a shaft 344 that defines (or is coupled to) a chamber 345 at a distal part of the shaft, the chamber being dimensioned to receive at least part of head 332. This, and/or one or more control rods 348 that are reversibly extendable from shaft 344 (e.g., medially and/or into chamber 345), can configure driver 242 to reversibly engage head 332, e.g., a driver interface 338 thereof. In some implementations, driver 242 (e.g., shaft 344) is dimensioned such that disposition of head 332 within chamber 345 maintains push-buttons 334 pushed-in medially, and thereby maintains lock 310 unlocked (FIG. 13A). In some implementations, exiting of head 332 from chamber 345 (e.g., upon withdrawal of driver 242 after anchor 330 has been anchored) allows spring 214 to relax (e.g., to decompress), transitioning lock 310 toward its locked state (FIG. 13B).

In some implementations, shaft 344 is a tube, with chamber 345 being a distal part of the lumen of the tube.

In some implementations, such as in some implementations in which tissue-engaging element 34 is a helical/screw-in tissue-engaging element, the tissue-engaging element can be rotatable independently of circumferential wall 316. That is, circumferential wall 316 can be rotatable with respect to tissue-engaging element 34 and/or interface 338. This enables tissue-engaging element 34 to be screwed into the tissue without wrapping tether 26 around the anchor, e.g., with the tether holding wall 316 stationary while the tissue-engaging element rotates. The driving (e.g., screwing) of tissue-engaging element 34 into the tissue can be performed by driver 342. Head 332 (e.g., interface 338 thereof) can be keyed with an anchor key 339, and driver 342 (e.g., shaft 244 and/or chamber 345) can be keyed with a drive key 346 that is complementary to the anchor key. Collectively, keys 339 and 346 thereby define a keyed joint via which driver 242 can apply torque to interface 338 of anchor 330, e.g., via rotation of shaft 344.

In some implementations, and as shown, anchor 330 (e.g., head 332 thereof) comprises a torque assembly 360 via which torque is transferred from interface 338 to tissue-engaging element 34. Torque assembly 360 can be disposed medially from circumferential wall 316, and can comprise one or more torque shafts that extend between interface 338 and tissue-engaging element 34. These torque shaft(s) can lie on central longitudinal axis ax2. In the example shown, torque assembly 360 comprises at least a first torque shaft 362 and a second torque shaft 364, which are operatively coupled to each other to transfer torque, e.g., due to their respective shapes rotationally locking them to each other. For example, and as shown, a noncircular and/or protruding part 365 of torque shaft 364 can cooperate with a noncircular and/or recessed part 363 of torque shaft 362 to define a keyed joint.

Thus, in some implementations, head 332 comprises a casing that has a proximal part (e.g., including interface 238), and a base part (e.g., including base plate 266) via which the head is coupled to tissue-engaging element 34. The proximal and distal parts of the head are axially separated at a mid-section at which circumferential wall 316 is disposed. Although the proximal part, the base part, and the tissue-engaging element are rotationally (and typically axially) locked with respect to each other, they are (e.g., collectively) rotatably coupled to circumferential wall 316. Lock 310 and/or circumferential wall 316 can axially traverse the mid-section.

In some implementations, and as shown, some components of head 323 can be common to both torque assembly 360 and lock 310. For example, and as shown, a unitary component 370 can be shaped to define surface 313, shaft 362, and press plate 312 (see FIG. 12C). Component 370 can be shaped and/or keyed to be rotationally locked to interface 338. For example, and as shown, surface 313 can be noncircular. In some implementations, shaft 362 thereby also serves to transfer the unlocking force that is applied to surface 313 by push-button(s) 334, e.g., the shaft pushing press plate 312 against spring 314, thereby compressing the spring between the press plate and base plate 366. Thus, in some implementations, component 370 can be considered to be a piston. In order to provide this functionality, the operative coupling between shafts 362 and 364 can allow shaft 362 to slide axially with respect to shaft 364. For example, and as shown, part 363 can be a groove or slit that runs substantially parallel with axis ax2, dimensioned to allow part 365 to slide axially therealong.

In some implementations, tether 26 extends laterally through head 332 by passing (i) into an aperture 318, (ii) across press plate 312 and/or past (e.g., at least partway around) torque assembly 360 (e.g., torque shaft 362 and/or torque shaft 364), and (ii) out of another aperture 318, e.g., similarly to as shown in FIG. 10A for anchor 230, mutatis mutandis.

As described hereinabove, driver 342 is reversibly engageable with interface 338. This engagement can be controlled via one or more control rods 348. For example, and as shown, while in an advanced position, control rods can protrude medially into chamber 345 in order to obstruct axial movement of anchor 330. In some implementations, and as shown, one or more of control rods 348 protrude distally from head 332 so as to obstruct movement of anchor 330 distally out of chamber 345. In some implementations, and as shown, one or more of control rods 348 protrude proximally from head 332 so as to obstruct movement of anchor 330 proximally within chamber 345. In some implementations, anchor 330 can be anchored to the tissue while control rods 348 protrude in this manner. In order to disengage driver 342 from anchor 330, one or more of control rods 348 (e.g., those that protrude distally from head 332) can be pulled proximally, such that they retract, e.g., into the wall of shaft 344.

In some implementations, control rod(s) 348 are circumferentially coincident with drive key(s) 346. In the example shown, two control rods 348 are disposed opposite each other, on the same plane on which two drive keys 346 lie. Moreover, in the example shown, control rods 348 extend medially out of drive keys 346. However, other alignments and arrangements of control rods 348 are possible, such as control rods 348 being disposed opposite each other on a plane that is orthogonal to the plane on which drive keys 346 lie.

In some implementations, and as shown, each push-button 334 is disposed at a respective anchor key 339, e.g., such that the drive key 346 that is complementary to the anchor key pushes/constrains the respective push-button medially.

FIGS. 14A-B are schematic illustrations of a system 400, in accordance with some implementations. System 400 is as described for system 300, mutatis mutandis, except where noted. A way in which system 400 differs from system 300 is its anchors 430, which can be as described for anchors 330, mutatis mutandis, except where noted.

Anchor 430 comprises a tissue-engaging element 34, and can also comprise a head 432 coupled to the tissue-engaging element (e.g., to a proximal end of the tissue-engaging element). In some implementations, the anchor (e.g., head 432) is slidably coupled, or is configured to be slidably coupled, to tether 26, e.g., in a similar way to head 332 of anchor 330, mutatis mutandis. Although in the example shown the tissue-engaging element of anchor 430 is tissue-engaging element 34 (described hereinabove), anchor 330 can alternatively or additionally comprise another tissue-engaging element, mutatis mutandis. In some implementations, head 432 comprises a lock that is configured, upon locking thereof, to lock the head to tether 26. In some implementations, this lock can share features with lock 210 or lock 310, mutatis mutandis.

Similarly to anchor 330, anchor 430 has a circumferential wall 416 that circumscribes (e.g., is coaxial with) a central longitudinal axis of the anchor, and that has one or more apertures 418 defined therein. In some implementations, head 432 is slidably coupled to tether 26 by the tether extending transversely through the head via apertures 418. However, whereas circumferential wall 316 is typically rotatable with respect to the tissue-engaging element and/or the interface of anchor 330, circumferential wall 416 is typically rotationally locked with respect to the tissue-engaging element and/or the interface of anchor 430. Thus, for anchor 430, screwing of tissue-engaging element 34 into the tissue also wraps tether 26 around circumferential wall 416, thereby tensioning the tether and contracting the tissue (FIG. 14B).

In some implementations, rather than being as described for anchor 330 except for circumferential wall 416 being rotationally fixed, anchor 430 is as described for anchor 230 except for circumferential wall 416 being rotationally fixed. For example, anchor 430 can be as described hereinabove, but with the interface and engagement by an anchor driver described for anchor 230.

FIGS. 15A-C and 16A-D are schematic illustrations of a system 500, in accordance with some implementations. System 500 comprises an implant 522, and a delivery tool 540 for percutaneous (e.g., transluminal, such as transfemoral) implantation of the implant. System 500 can be as described for system 200, mutatis mutandis, except where noted. For example, anchors 530 and driver 542 of system 500 are different to those of system 200. However, the functionality of system 500, and the techniques for use therewith, are generally similar to those of system 200. For example, system 500 allows tether 26 to be tensioned after the anchoring of each anchor, and for that tension to be fixed by locking the head of the anchor to the tether, e.g., by locking a lock of the head. Although only a single anchor 530 is shown, system 500 can comprise multiple anchors 530, e.g., as described for systems 200, 300, and 400, mutatis mutandis. In the example shown, the tether of implant 222 is tether 26 (described hereinabove), but implant 522 can alternatively or additionally comprise another tether, mutatis mutandis.

Although FIG. 15A shows tether 26 separate from anchor 530 of implant 522, the implant can be provided with multiple anchors 530 already connected to the tether, e.g., as described in International Patent Application PCT/IB2021/058665 to Halabi et al., which published as WO 2022/064401, and/or International Patent Application PCT/IB2022/051099 to Shafigh et al., mutatis mutandis. In some implementations, implant 522 can be provided with one or more of anchors 530 separate from tether 26, and configured to be connected to the tether.

FIG. 15B shows anchor 530 in cross-section, and FIG. 15C shows an exploded view of the anchor. Anchor 530 comprises a tissue-engaging element 34, and can comprise a head 532 coupled to the tissue-engaging element (e.g., to a proximal end of the tissue-engaging element). Although in the example shown the tissue-engaging element of anchor 530 is tissue-engaging element 34 (described hereinabove), anchor 530 can alternatively or additionally comprise another tissue-engaging element, mutatis mutandis.

Head 532 is slidably coupled, or is configured to be slidably coupled, to tether 26. For example, and as shown, head 532 (e.g., a casing of the head) can comprise or define a circumferential wall 516 that circumscribes (e.g., is coaxial with) a central longitudinal axis ax3 of anchor 530, and that has one or more apertures 518 defined therein. Head 532 can be slidably coupled to tether 26 by the tether extending transversely through the head via apertures 518 (FIGS. 16A-D).

Delivery tool 540 is configured to transluminally advance anchors 530 (and thereby typically implant 522 in general) to the heart of the subject. Delivery tool 540 can comprise a driver 542. Delivery tool 540 can also comprise a flexible tube (e.g., a catheter) 550 that is transluminally advanceable to the heart, and through which driver 542 is extendable. Delivery tool 540 can be configured to, for each anchor 530 sequentially, (i) anchor the anchor to the tissue, such that a section of tether 26 extends to the anchor from a preceding one of the anchors (i.e., the anchor to have been most recently anchored), subsequently adjust tension on the section of the tether, and subsequently fix the tension by locking a lock 510 of the anchor.

In some implementations, implant 522 can comprise a leading anchor, e.g., similar to that of other systems described herein, mutatis mutandis. In some implementations, driver 542 (e.g., delivery tool 540) is also configured to anchor the leading anchor. For example, a driver interface of the head of the leading anchor can be similar driver interface 538 of anchors 530, described hereinbelow. In some implementations, a separate driver (e.g., a separate delivery tool) can be provided for the leading anchor.

The lockability of anchor 530 is provided by a set screw 513 that is mounted (e.g., within the casing of head 532, which can have a complementary inner thread) such that rotation of the set screw moves a press surface 511 (e.g., defined by the set screw) toward an opposing surface 566. In the example shown, opposing surface 566 is an internal of head 532 and, as shown, can be defined by base plate of the head. While tether 26 extends through head 532, the tether extends between press surface 511 and opposing surface 566, such that the movement of the press surface toward the opposing surface clamps the tether therebetween, thereby locking anchor 530 to the tether. Thus, set screw 513, press surface 511, and opposing surface 566 can be considered to be components of a lock 510 of head 532.

Lock 510 can be locked by a force applied by driver 542. For example, driver 542 can apply torque to set screw 513 (e.g., via engagement with a recess/slot of the set screw, e.g., an inner surface thereof). Similarly, driver 542 can apply reverse torque to unlock lock 510. In the example shown, the torque is applied by a control rod 548 of driver 542. For example, while driver 542 is engaged with anchor 530, keying (e.g., a protrusion) 549 of control rod 548 can extend into a recess of set screw 513, such that rotation of the control rod applies torque to the set screw.

In some implementations, such as in some implementations in which tissue-engaging element 34 is a helical/screw-in tissue-engaging element, the tissue-engaging element can be rotatable independently of circumferential wall 516. This enables tissue-engaging element 34 to be screwed into the tissue without wrapping tether 26 around the anchor, e.g., with the tether and/or driver 542 holding wall 516 stationary while the tissue-engaging element rotates. The driving (e.g., screwing) of tissue-engaging element 34 into the tissue can be performed by driver 542. Driver 542 can comprise a drive head 546, and a shaft (e.g., a driveshaft) 544 that extends from an extracorporeal portion (e.g., a control handle) to the drive head. Drive head 546 is reversibly engageable with a driver interface 538 of head 532 of anchor 530, and together can be considered to define a torque assembly. FIG. 16A shows anchor 530 being brought close to tissue 10 by driver 542.

In some implementations, and as shown (e.g., in FIGS. 16A-B), tether 26 extends laterally through head 532 by passing (i) into an aperture 518, (ii) across press surface 511 and/or opposing surface 566, and/or past (e.g., at least partway around) interface 538 and/or drive head 546, and (ii) out of another aperture 218.

FIG. 16B shows anchor 530 (e.g., tissue-engaging element 34 thereof) having been screwed into tissue 10 while tether 26 extends from a previously-anchored anchor, laterally through head 532, without the tether becoming wrapped around head 532, e.g., by circumferential wall 516 (and thereby apertures 518) remaining stationary during rotation of tissue-engaging element 34. As described hereinabove, the torque for this screwing can be provided by rotation of shaft 544, while drive head 546 is engaged with driver interface 538. This rotation of shaft 544 can be relative to control rod 548, which can hold set screw 513 from rotating with respect to circumferential wall 516, which itself can be held from rotating with respect to the tissue by tether 26.

FIG. 16C shows lock 510 having been locked by control rod 548 rotating set screw 513 (e.g., with respect to the casing of head 532, e.g., circumferential wall 516 thereof), such that tether 26 becomes clamped between press surface 511 and opposing surface 566. This locking can be performed after a desired degree of tension has been applied to tether 26, e.g., as described hereinabove for other systems, mutatis mutandis. During this locking, shaft 544 can hold interface 538 (and thereby tissue-engaging element 34) from rotating with respect to the tissue. Subsequently, driver 542 can be disengaged from anchor 530 and withdrawn (FIG. 16D).

It is to be noted that interface 538 is disposed distally from (i.e., closer to tissue-engaging element 34 than) lock 510. It is also to be noted that shaft 544 extends distally beyond lock 510 (e.g., set screw 513) to where drive head 546 engages interface 538. As shown, control rod 548 can extend through set screw 513 (e.g., an axial channel in the set screw) in a manner that does not engage the set screw, such that it can apply torque to tissue-engaging element 34 without rotating the set screw.

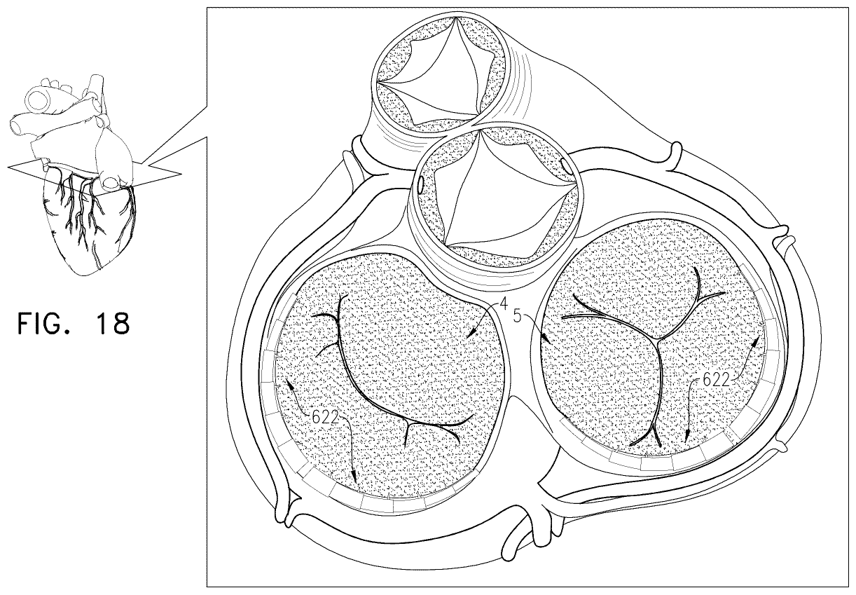

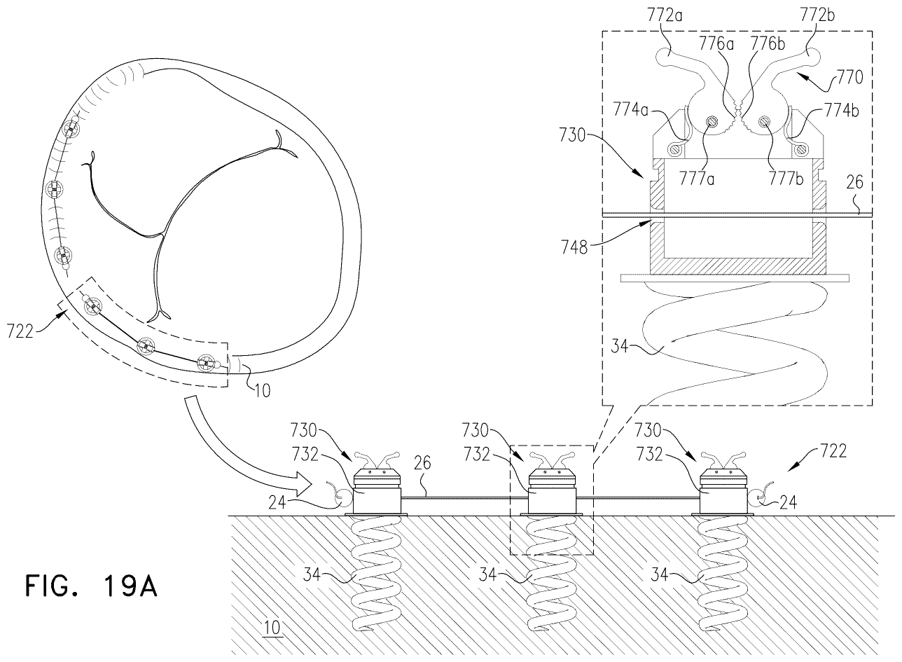

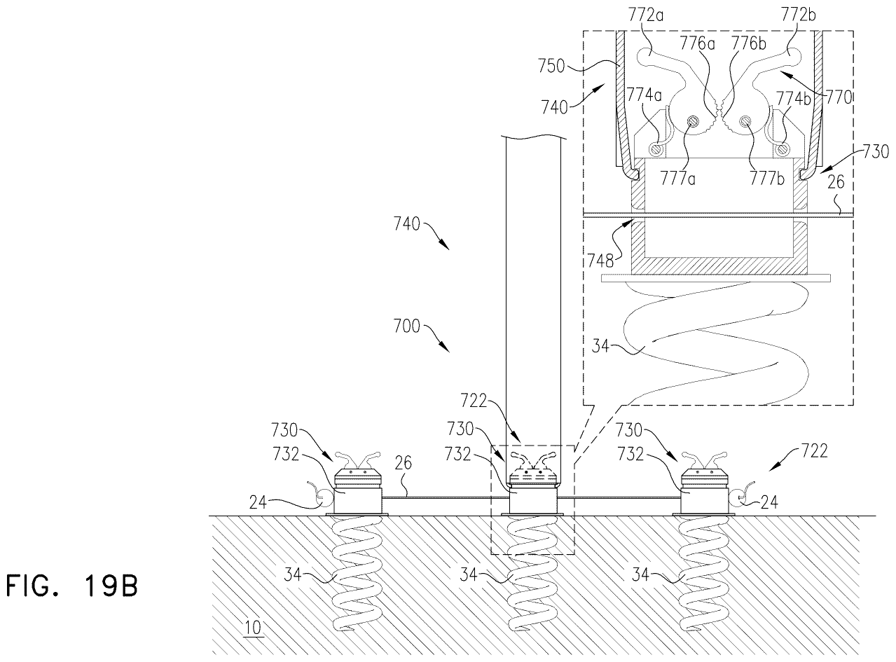

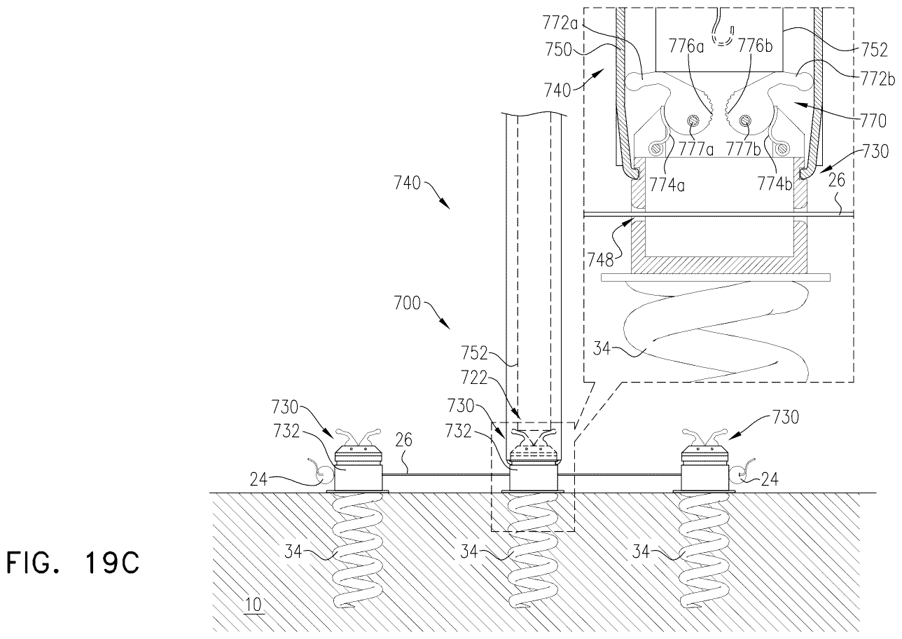

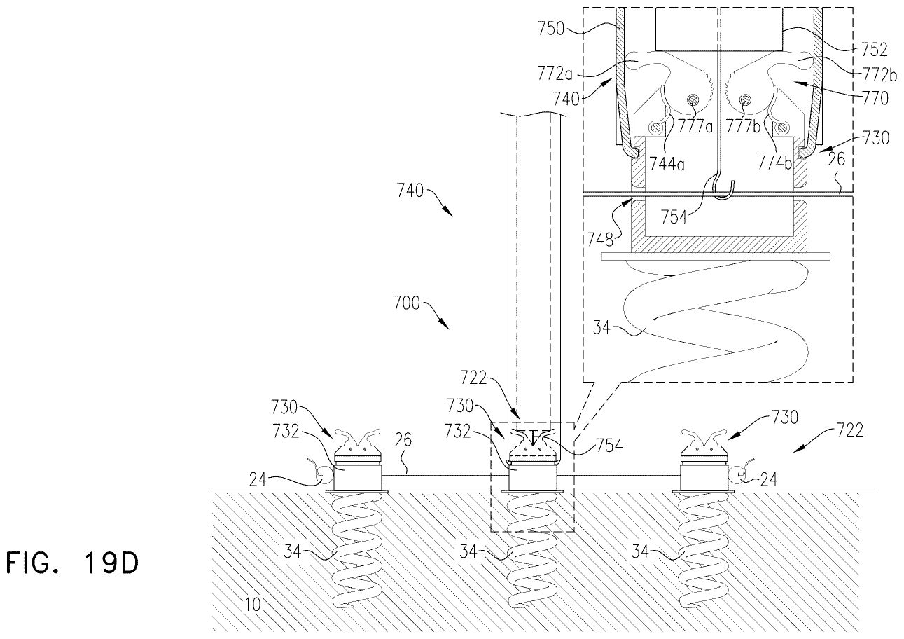

Reference is now made to FIGS. 17A-E and 18, which are schematic illustrations of a system 600 and techniques for use therewith, in accordance with some implementations. System 600, and the techniques for its use, share features with other systems described hereinabove (e.g., systems 200, 300, 400, and/or 500). An example of such a shared feature can be the iterative process of securing a section of an implant (e.g., a section that extends between two anchors) to the tissue, contracting that section, and then doing the same for a subsequent section. However, rather than providing an implant that comprises anchors that are threaded on a tether, system 600 provides a segmented implant that is telescopically extendible and contractible.

System 600 comprises an implant 622, and can also comprise a delivery tool 640 for percutaneous (e.g., transluminal, such as transfemoral) implantation of the implant. Implant 622 is a segmented implant, comprising multiple segments 624, telescopically coupled to each other in series, each of the segments comprising an anchor 630. Delivery tool 640 is configured to percutaneously deliver implant 622 to the heart, to anchor each of anchors 630 to tissue 10 of the heart (e.g., an annulus of a valve, such as tricuspid valve 5, as shown), and to contract the tissue by telescopically contracting the implant while each of the anchors remain anchored to the tissue. In some implementations, this is performed in a successive manner, whereby a section 626 of the implant (e.g., extending between two anchors 630, and/or overlapping two segments 624) is secured to the tissue (e.g., by anchoring both of the two anchors between which the section extends), subsequently contracted telescopically, and can be locked, prior to the same being done with a successive section of the implant.

A first segment 624a of implant 622 is secured to tissue 10 by anchoring, to the tissue, an anchor 630 of the segment (FIG. 17A). In some implementations, anchor 630 becomes deployed automatically upon exposure from delivery tool 640. In some implementations, a discrete driver (not shown) can be disposed within implant 622 (e.g., extending into segments 624, such as into lumens defined by the segments), and can control deployment of anchors 630 independently of their exposure from the delivery tool. For example, the driver can initially constrain anchor 630, and can be operated to release the anchor at the desired time. That is, the anchor 630 can be biased toward assuming an anchoring position, but the delivery tool constrains the anchor in a delivery position during delivery of the implant. In some implementations, each anchor 630 can be constrained in the delivery position by an adjacent segment 624 (e.g., the previously-anchored segment, or the segment that is the next to be anchored). For example, the anchor can be constrained in the delivery position by being disposed within the adjacent segment, and the delivery tool can release the anchor by telescopically extending the segment out of the segment that is constraining the anchor.

Subsequently, a second segment 624b is telescopically extended from segment 624a (FIG. 17B), and its anchor 630 is anchored to the tissue (FIG. 17C). Thus, FIGS. 17A-C show a first section 626a of implant being secured along a portion of a path of tissue 10. First section 626a includes part of segment 624a and part of segment 624b. Subsequently, a desired degree of contraction is applied to the portion of the path along which first section 626a is secured, by reducing the length of section 626a (FIG. 17D). As shown, this can be achieved by telescopically contracting segment 624b into segment 624a. This reduced length (and thereby this desired degree of contraction) is fixed by locking segment 624a to segment 624b (i.e., fixing an amount of telescoping between the segments). This locking may be performed as a discrete action following the reduction of the length (e.g., by locking a lock), or may be intrinsic to the manner in which the length is reduced, e.g., one or both of segments 624a and 624b include a ratchet and/or a thread that prevents passive re-increasing of the length.

In some implementations, the contraction of section 626a of implant 622 is performed by one component of delivery tool 640 pulling on segment 624b while another component of the delivery tool pushes on segment 624a. In some implementations, implant 622 comprises a tether that extends along (e.g., through) the implant, and which is tensioned each time a section 626 is to be contracted, e.g., similarly to as described hereinabove for other implants, mutatis mutandis.

These steps are repeated iteratively for each section 626 of the implant, using further segments 624. FIG. 17E shows implant 622 having six segments (segment 624a, segment 624b, segment 624c, segment 624d, segment 624c, and segment 624f), defining five sections (section 626a, section 626b, section 626c, section 626d, and section 626c). In the example shown, section 626a is secured along a portion 10i of tissue 10 (e.g., of the path along the tissue); sections 626b and 626c are secured along a portion 10j of the tissue; section 626d is secured along a portion 10k of the tissue, and section 626e is secured along a portion 101 of the tissue. Portions 10i and 101 are shown as having been contracted to similar degrees of contraction, portion 10k is shown as having been contracted to a lesser degree than portions 10i and 101 (e.g., as not having been contracted at all), and portion 10j is shown as having been contracted to a greater degree than portions 10i and 10l. It is to be understood that the particular implantation arrangement of implant 622 and the degrees of contraction of the various portions of the tissue are intended to be illustrative examples, and not limiting.

In some implementations, each segment 624 of implant 622 is articulatably coupled with respect to its adjacent segments, at least during implantation, thereby allowing the implant to be implanted along a curved path, such as around an annulus of a valve, as shown.

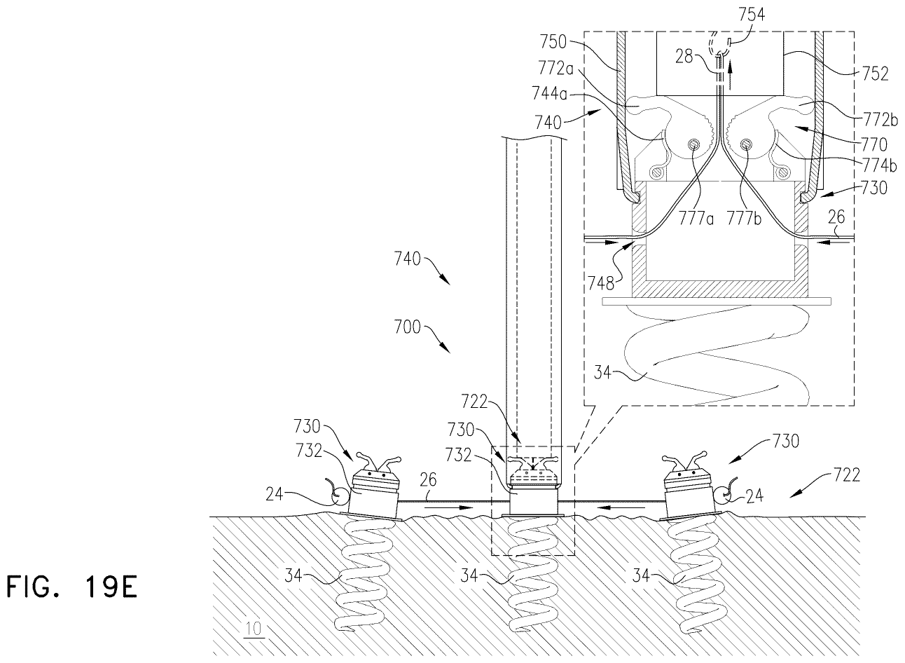

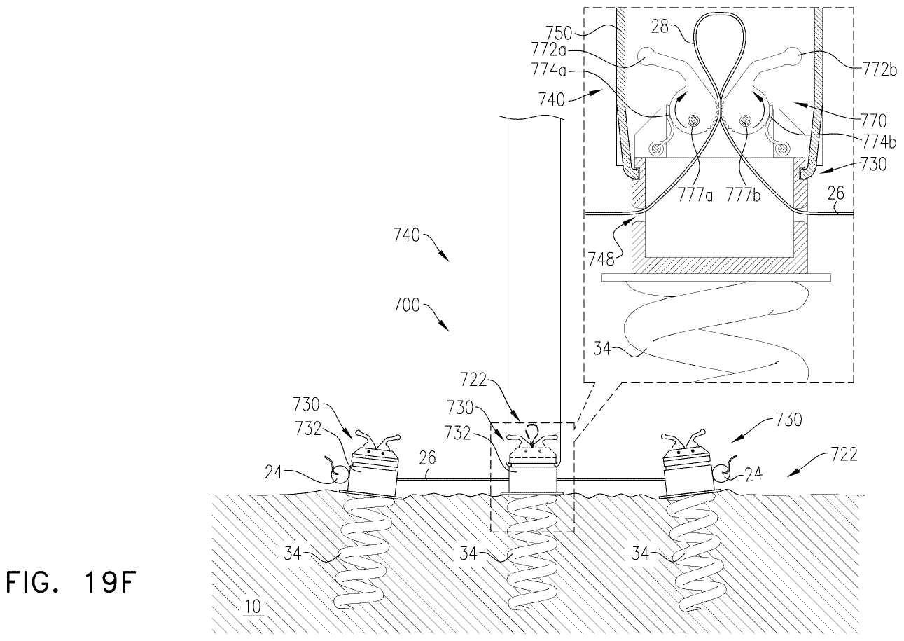

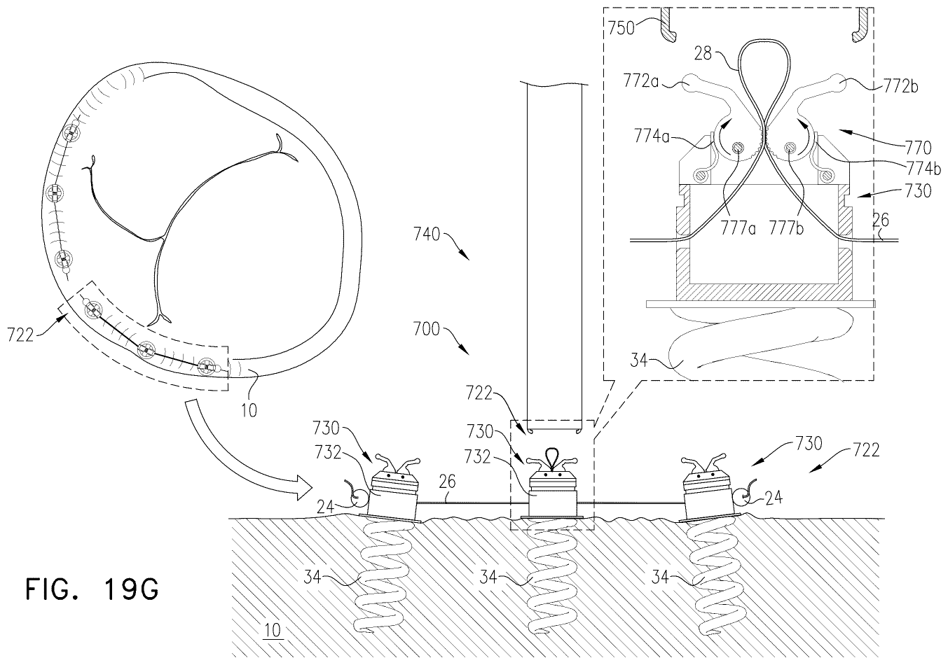

Reference is made to FIGS. 19A-G, which are schematic illustrations of a system 700 and techniques for tensioning an implant 722 of the system, in accordance with some implementations. Similarly to system 20, system 700 is a tissue-adjustment system and can be used for adjusting a dimension of a tissue 10, such as a tissue of the heart of a subject. In some implementations, system 700 is adapted to allow for tensioning of a tether of the implant once the implant has been fully implanted within the heart, e.g., once all the anchors of the implant have been anchored to the tissue and/or once the tether has been trimmed and locked to the implant (FIG. 19A).

In some implementations, system 700 can be an annuloplasty system, and implant 22 can be an annuloplasty structure (e.g., an annuloplasty ring or band) for implanting at (e.g., along) an annulus of a valve of the heart. Implant 722 can comprise multiple anchors 730 and tether 26 to which the anchors are slidably coupled, e.g., by being threaded on the tether.

Each anchor 730 comprises a head 732, and a tissue-engaging element 34 extending distally away from the head. Tissue-engaging element 34 defines an anchor axis of the anchor, i.e., an axis along which the anchor is advanced during anchoring.

In some implementations in which tissue-engaging element 34 is substantially linear (e.g., a dart, etc.), it may lie on the anchor axis. In some implementations in which tissue-engaging element 34 is helical, the anchor axis may be the helix axis of the tissue-engaging element, e.g., as shown.

In some implementations, tether 26 extends through head 732, e.g., through a horizontal channel 748 defined by the head that is transverse to the anchor axis. In some implementations, tether 26 is slidable through channel 748.

Similarly to systems 200 and 300, in some implementations, each head 732 comprises a lock 770 that is configured, upon locking thereof, to lock the head to tether 26.

In some implementations, lock 770 can comprise a first locking element 772a and a second locking element 772b, adapted to lock head 732 to tether 26 by trapping the tether between the locking elements. The first locking element and the second locking element can be configured in a variety of ways. In some implementations, the locking elements can be the same as or similar to one or more of levers, ratchets, teeth, barbs, feet, pincers, beads, crimpers, etc.

In some implementations, lock 770 is biased to assume a locked state in which the lock is locked to the tether if the tether is disposed between the locking elements. This biasing can be achieved by locking elements 772a and 772b being biased to pivot in a manner that reduce the size of a gap between them.

In some implementations, each locking element 772a and 772b has an associated spring 774a and 774b, that is adapted to bias the locking element towards the locked state, e.g., in the absence of an unlocking force being applied to the lock.

In some implementations, each of locking elements 772a and 772b defines a trapping surface 776a and 776b, respectively. In some implementations, the locking elements are biased to pivot in a manner that reduces the size of the gap between the trapping surfaces. The trapping surfaces may be knurled and/or may define a plurality of teeth, to facilitate engagement with tether 26. In some implementations, the locking elements are configured such that while lock 770 is locked to tether 26, the lock has a ratchet-like nature, e.g., facilitating movement of the tether between the locking elements in only one direction. In some implementations, and as shown, the lock may even increase its grip on tether 26 in response to pulling of the tether in the other direction (e.g., in the direction that would otherwise release the tether from the lock). In the example shown, this is achieved by the geometry of the locking elements and their respective hinge points 777a and 777b via which they are mounted, e.g., eccentric positioning of the hinge point with respect to the position and/or the curvature of the trapping surface.

System 700 can comprise, in addition to implant 722, a tensioning tool 740 for percutaneous (e.g., transluminal, such as transfemoral) tensioning of the implant. In some implementations, tool 740 is adapted to be advanced and engaged with any anchor 730 of implant 722 subsequently to implantation of the implant within the heart (e.g., once all, or at least some of, the anchors of the implant have been implanted at the tissue). Thus, system 700 may advantageously allow adjustment (e.g., increasing) of tension on tether 26 should it be determined that this would be desirable subsequently to the original locking of tension in the tether.

In some implementations, tool 740 comprises a catheter 750 (e.g., a transluminal catheter) adapted to engage with anchor head 732 of anchor 730 (e.g., as shown in FIG. 19B). Tool 740 can comprise an unlocker 752, which can comprise a tube. Unlocker 752 can be disposed within and/or advanceable through catheter 750. Unlocker 752 can be adapted to transition lock 770 towards an unlocked state, e.g., by applying an unlocking force to the lock.

Tool 740 comprises a grasper 754 (e.g., a hook, jaws, or a clamp), adapted to grasp tether 26, and to increase tension on the tether by pulling the tether through the gap between locking elements 722a and 722b (FIG. 19D). As shown, grasper 754 can be adapted to be advanced through catheter 750 and/or through unlocker 752.

FIGS. 19B-G represent a series of steps that may be performed by the operator, to apply tension to implant 722. It is to be noted that FIGS. 19A-G are primarily intended to illustrate the capability of system 700, rather than to strictly define a sequence of steps of a procedure.

FIG. 19A shows implant 722 having been implanted in the heart, with anchors 730 anchored to tissue 10 (e.g., to the annulus of a valve of the heart). FIG. 19A schematically illustrates tension on tether 26 being insufficient to optimally contract tissue 10. This insufficiency may have occurred during implantation or may have developed since implantation.

Subsequently to implantation of implant 722 (e.g., during the same medical procedure, or during a subsequent medical procedure), catheter 750 is advanced to the heart, and engaged with an anchor head 732 of an anchor 730 of the implant (FIG. 19B). Unlocker 752 is then used to transition lock 770 towards the unlocked state, e.g., by pressing against locking elements 772a and 772b (e.g., against levers defined by the locking elements) (FIG. 19C). In some implementations, unlocker 752 is advanced to lock 770 via an opening defined by anchor head 732 that provides the unlocker with access to the lock, e.g., as shown. In some implementations, unlocker 752 transitions the lock towards the unlocked state by causing locking elements 772a and 772b to pivot away from each other, thus increasing the size of a gap therebetween, as shown. While the lock remains in the unlocked state (e.g., while unlocker 752 remains against lock 770), grasper 754 is advanced through the gap between the locking elements, to engage tether 26 within channel 748, e.g., the grasper grasps (e.g., hooks onto) the tether (FIG. 19D).

Grasper 754 can then be pulled proximally through lock 770 (e.g., through the gap between the locking elements), thus forming a loop 28 in the tether (FIG. 19E). As shown in FIG. 19E, this tensions implant 722, reducing the distance between anchors 730 (e.g., by the tether pulling the anchors toward each other), thus contracting tissue 10 of the heart.

In order to lock the tension in the implant, unlocker 752 can then be retracted (FIG. 19F), thereby allowing lock 770 to transition towards its locked state, e.g., by locking elements 772a and 772b pivoting inwardly towards each other and clamping the tether therebetween. In this state, tether 26 (e.g., loop 28 thereof) is arranged such that the tether extends away from channel 748 and through lock 770, and loops back through the lock to the channel.

Catheter 750 can then be retracted (FIG. 19G), leaving implant 722 implanted within tissue 10, in the tensioned state.

FIGS. 19A-G show implant 722 having three anchors, but it is to be understood that implant 722 can have more or fewer anchors. In some implementations, the abovementioned technique for applying tension to an anchor 730 of implant 722 is performed on an as-needed basis, e.g., according to intra-procedural imaging. Thus, only one or a selected subset of anchors 730 might be tensioned in this manner. In some implementations, all the anchors of the implant can be tensioned sequentially in this manner.

It is to be further noted that anchor 730 and/or catheter 750 can be used in combination with other systems and/or implants described herein and/or in International Patent Application PCT/IB2022/051099 to Shafigh et al., filed Feb. 8, 2022, which is incorporated herein by reference. For example, one or more anchors of such an implant can be replaced with an anchor 730, in order to provide the option of post-implantation adjustment described herein.

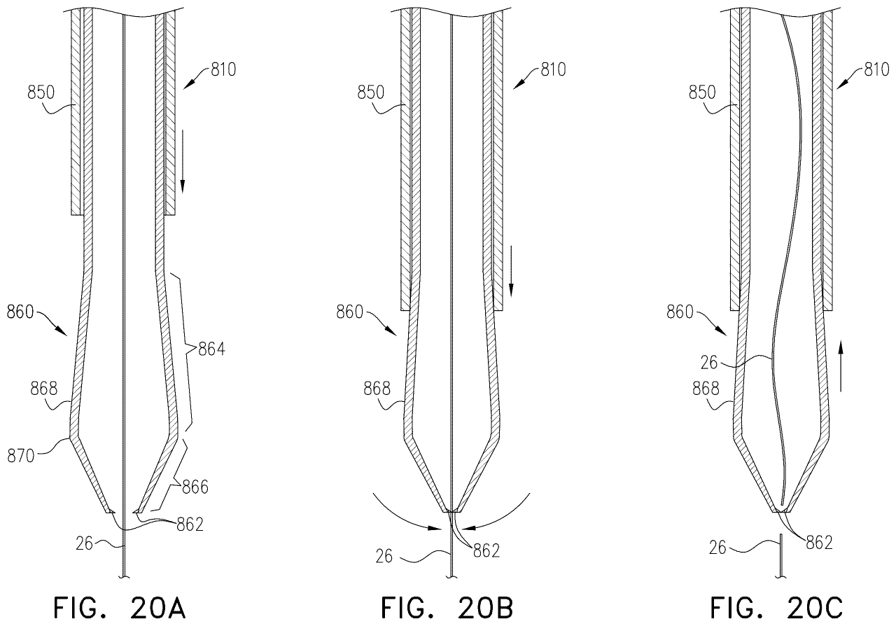

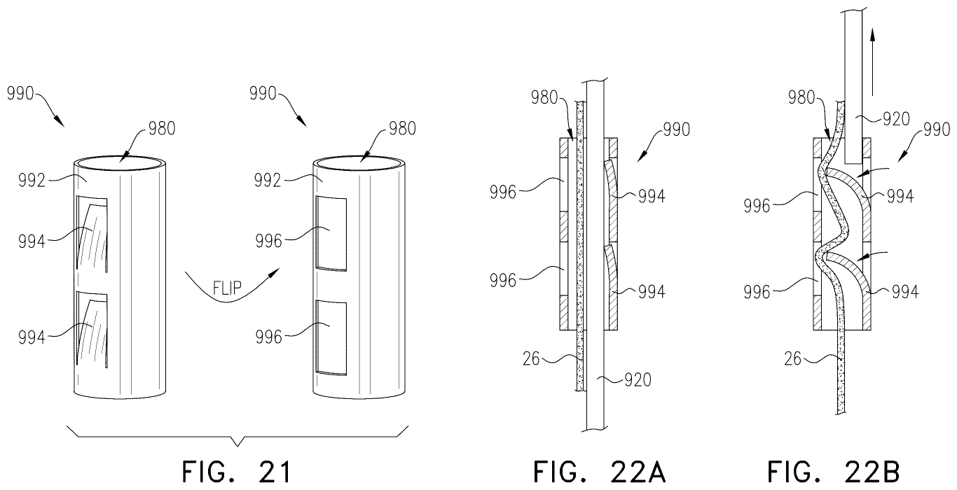

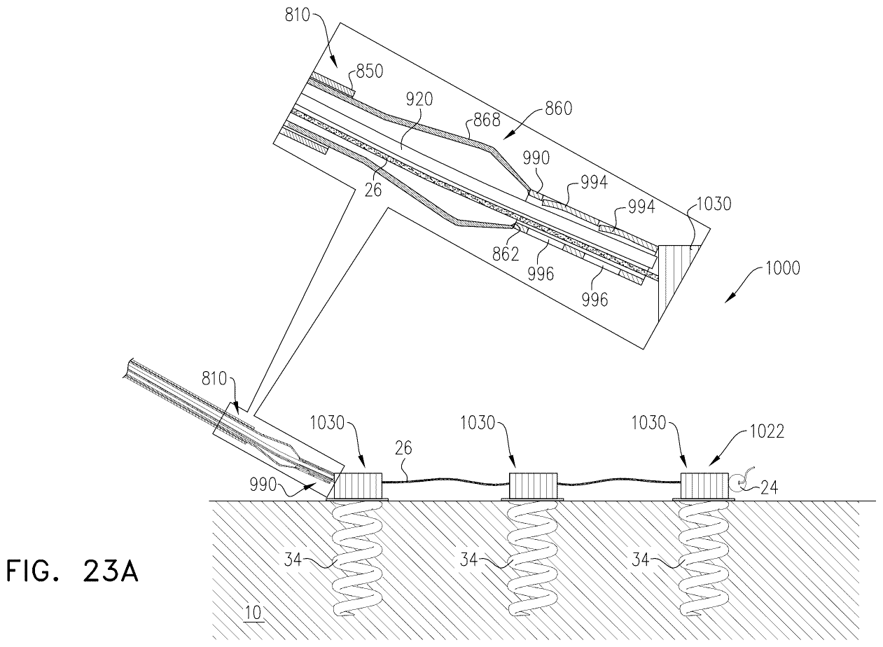

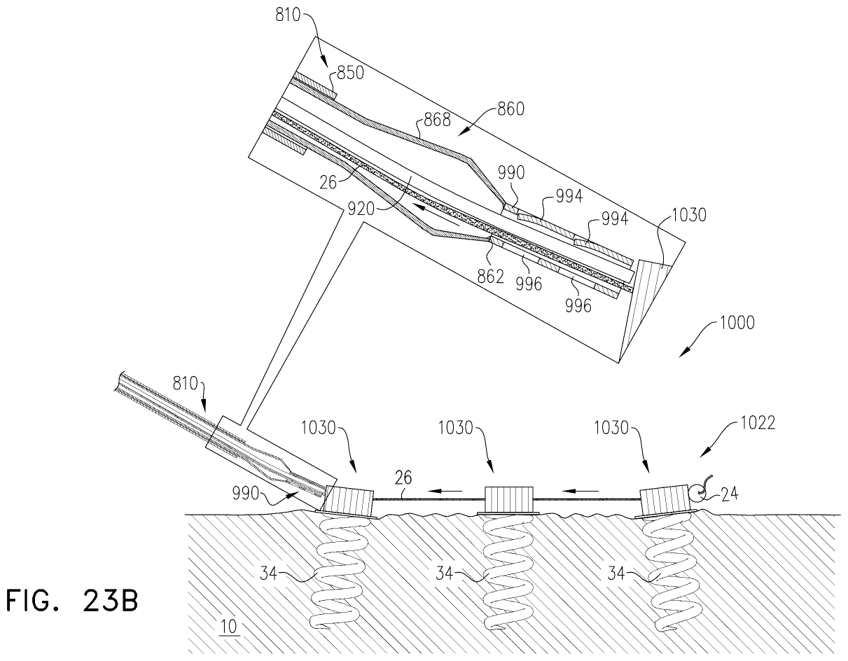

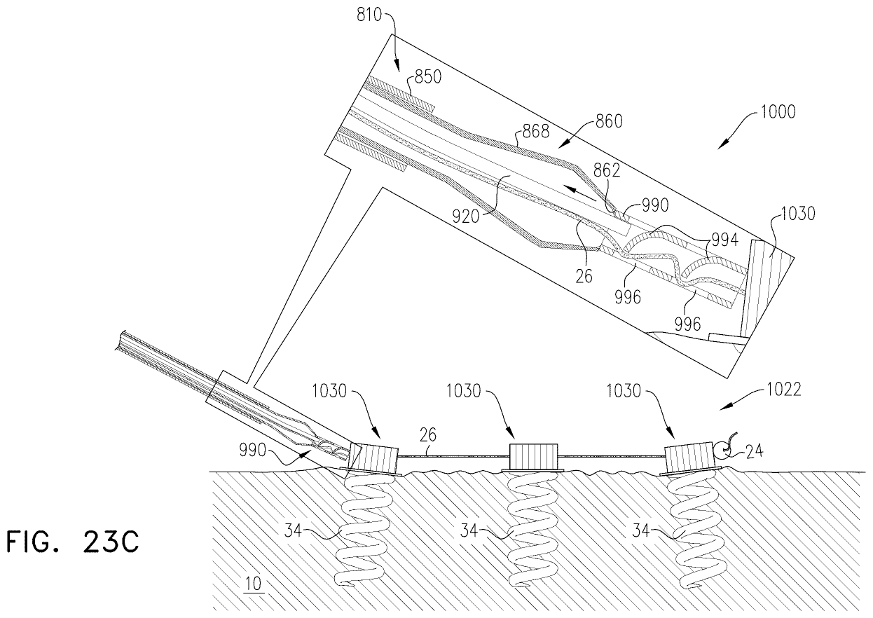

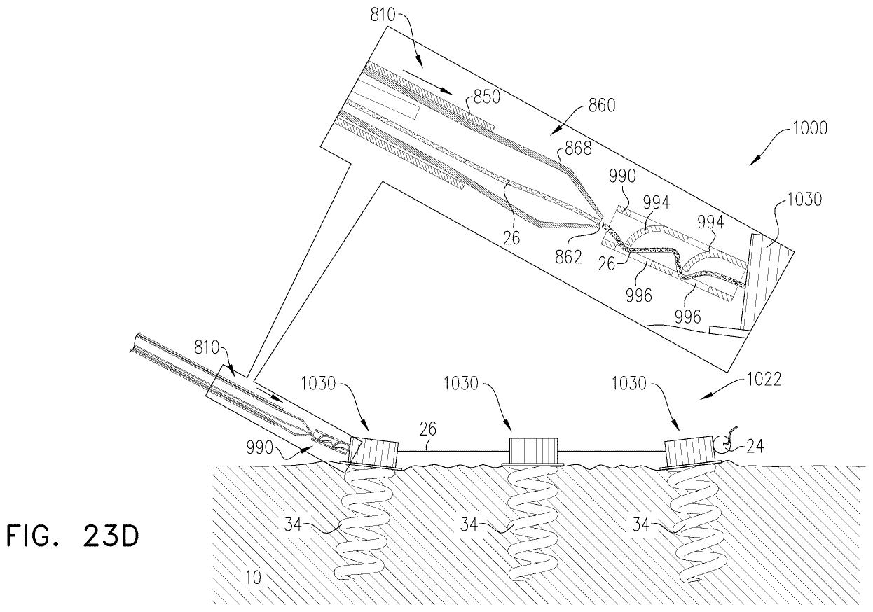

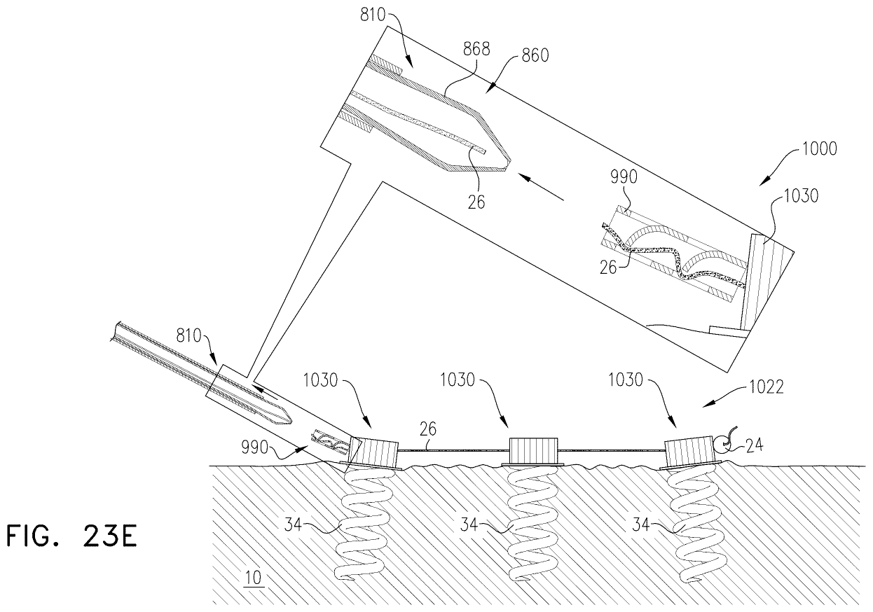

Reference is now made to FIGS. 20A-C, 21, 22A-B, and 23A-E, which are schematic illustrations of apparatus for use with a tether, in accordance with some implementations. FIG. 20A-C show a cutter 810 for use with a tether, in accordance with some implementations. FIG. 21 and FIGS. 22A-B show a lock 990 for use with a tether, in accordance with some implementations. FIGS. 23A-E show a system 1000, comprising cutter 810 and lock 990, being used with tether 26, in accordance with some implementations.

Lock 990 can be a variant of, or used instead of, lock 24 mentioned hereinabove. As mentioned with reference to lock 24, lock 990 can be locked onto a tether of an implant (e.g., tether 26 of any of the implants mentioned hereinabove), in order to maintain tension in the implant.

Lock 990 comprises a tubular wall 992, that circumscribes a lumen 980 dimensioned to receive a tether (e.g., tether 26) therethrough. A window 994 (e.g., a hole, aperture or opening) is cut out of the wall and positioned on an opposite side of the wall to the window, is a tab 996 (e.g., having only a single edge attached to wall 992) also cut from the wall. In some implementations, tab 996 is biased (e.g., shape-set) to deflect across lumen 980 and protrude into window 994 in a manner that locks the lock to the tether.

In some implementations, and as shown, wall 992 defines multiple windows 994 cut out of the wall, and multiple corresponding tabs 996 cut from the wall, each tab facing a respective window and adapted to deflect across the lumen and into the respective window, in order to trap the tether within the lock.

In some implementations, lock 990 is advantageously manufactured simply by cutting it from a single piece of stock tubing (e.g., nitinol tubing), and shape-setting tab(s) 996.

In some implementations, an obstructing rod 920 is adapted to maintain the lock in an unlocked state in which the tab is prevented from deflecting across the lumen and into the window (FIG. 22A). In some implementations, in the unlocked state of the lock, the rod extends through the lumen alongside the tether, thus shielding the tether from the tab(s). In some implementations, withdrawing the rod from the lock transitions the lock towards a locked state by allowing the tab(s) to deflect across the lumen and pushing the tether into the window (FIG. 22B).

It is to be noted that, although rod 920 can have a circular cross-section, it can alternatively have an elongate (e.g., rectangular) cross section, similar to a ribbon, or a curved cross-section (e.g., an arc), e.g., in order to facilitate shielding of tether 26.

FIGS. 20A-20C show cutting of tether 26 of an implant, in accordance with some implementations. In some implementations, and as will be explained hereinbelow with reference to FIGS. 23A-D, cutter 810 can be configured to be used along with lock 990, e.g., in order to lock and cut the tether. Cutter 810 comprises a cutting element 860 adapted to cut tether 26. Cutter 810 can comprise one or more of a variety of cutting elements, e.g., blade, knife, scissors, pincer, razor, scalpel, edge, sharp edge, cutting edge, etc.

In some implementations, cutter 810 (e.g., cutting element 860 thereof) can define a lumen dimensioned to receive the tether therethrough.

In some implementations, at a distal end of cutting element 860, the cutting element defines a pair of cutting edges 862 facing each other, such that tether 26 can extend therebetween (FIG. 20A). Cutter 810 further comprises an overtube 850 that is slidable over and along cutting element 860 in a manner that urges the cutting edges towards each other, thereby cutting the tether (FIGS. 20B-C).

In some implementations, cutting element 860 comprises a pair of arms 868, each arm having a proximal part 864 that extends distally and away from the proximal part of the other arm of the pair and, a distal part 866 that extends distally from the proximal part and towards the distal part of the other arm of the pair. In the example shown, each arm 868 has a discrete elbow 870 between proximal part 864 and distal part 866. However, it is to be understood that in some implementations the arm may not have a discrete elbow (e.g., may define a continuous curve from the proximal part to the distal part).

Cutting edge 862 can be coupled to or defined by distal part 866 of the arm, e.g., as shown in FIG. 20A-C. In some implementations, at rest, proximal parts 864 extend outwardly such that arms 868 collectively define a wider diameter d3 than an inner diameter of overtube 850, such that sliding the overtube distally over and along proximal parts 864 squeezes together the proximal parts, causing the arms to pivot inwardly towards each other, and cutting edges 862 to cut tether 26 (FIGS. 20B-C). In some implementations, and as shown in FIG. 20A, at rest, an angle at which proximal part 864 is disposed with respect to a central axis of the cutter is shallower than an angle at which distal part 866 is disposed with respect to the central axis. In some implementations, and as shown, proximal part 864 is longer than distal part 866.

In some implementations, cutting element 860 is advantageously manufactured simply by cutting it from a single piece of stock tubing (e.g., nitinol tubing), and shape-setting the arms and the cutting edges. Similarly, overtube 850 can be a simple tube. That is, in some implementations, cutter 810 can consist essentially of two concentric tubes, the inner of which has been cut and shape set to define arms and/or cutting edges. In some implementations, this advantageously allows cutter 810 to be sufficiently small in size for transluminal delivery to the heart (e.g., having a diameter that is sufficiently small to fit through other components of a delivery system). For example, whereas adjustment tool 60 is shown/described as being advanced towards an implant after tube 50 is withdrawn from the subject, cutter 810 can be advanceable through such a tube as tube 50. In some implementations, this may allow for subsequent introduction of more anchors. For example, this may facilitate the implantation of multiple implants (e.g., the techniques described with reference to FIGS. 3 and 4) without necessitating the removal of tube 50. In another example, cutter 810 can be used to advance a lock 990 after a subset of anchors have been anchored, but prior to the anchoring of a further subset of anchors e.g., using a technique similar to the technique shown in FIGS. 7A-F. That is, in some implementations and/or for certain steps, cutter 810 can be used to advance an anchor but not to cut the tether. Such advantages can be similarly conferred, mutatis mutandis, by lock 990 being cut from a single piece of stock tubing.

Cutting element 860 can be advanced to the heart over and along tether 26 (i.e., while tether 26 extends through the cutting element). In some implementations, cutter 810 is advanced to the heart by advancing cutting element 860 together with overtube 850 over and along the tether, e.g., without advancing the overtube with respect to the cutting element until it is desired to cut the tether.

Reference is now made to FIGS. 23A-E, which are schematic illustrations of a system 1000 for locking and cutting a tether 26 of an implant 1022, in accordance with some implementations. In some implementations, implant 1022 is a variant of, or substantially identical to, any of the implants described hereinabove, comprising multiple anchors 1030, which can be a variant of any of the anchors described hereinabove. System 1000 can comprise cutter 810 and lock 990 in order to lock and cut the tether.

In some implementations, cutter 810 is adapted to transluminally advance (e.g., push) lock 990 to implant 1022 implanted at the heart. For example, and as shown, cutter 810 can be advanced to the heart with a distal part of cutter 810 (e.g., a distal part of cutting element 860) abutting a proximal end of the lock, such that advancing cutter 810 distally towards implant 1022 implanted at the heart pushes lock 990 distally towards the implant.

In some implementations, lock 990 is advanced to the implant with obstructing rod 920 extending through the lock, in order to maintain the lock in the unlocked state (FIG. 23A). In some implementations, obstructing rod 920 can extend from lock 990 proximally through cutter 810 (e.g., through cutting element 860), to outside of the subject. In some implementations, tether 26 can be tensioned while the tether extends through lock 990 (FIG. 23B), e.g., by pulling the tether proximally from outside of the subject. A reference force can be provided by via cutter 810 and lock 990 (e.g., by maintaining the lock abutting a proximal-most anchor 1030).

Subsequently (e.g., once tether 26 has been adequately tensioned), obstructing rod 920 is withdrawn from lock 990 (e.g., by pulling the rod proximally from outside the subject), such that tab(s) 994 deflect across lumen 980 and into window(s) 996, thereby locking the lock to tether 26 (FIG. 23C).

With lock 990 locked to tether 26, the tether can then be cut, e.g., by sliding overtube 850 distally over cutting element 860, thereby causing cutting edges 862 to move towards each other and sever the tether (FIG. 23D).

Cutter 810 can then be withdrawn out of the subject, leaving implant 1022 implanted at the heart, with lock 990 maintaining tension in tether 26 (FIG. 23E).

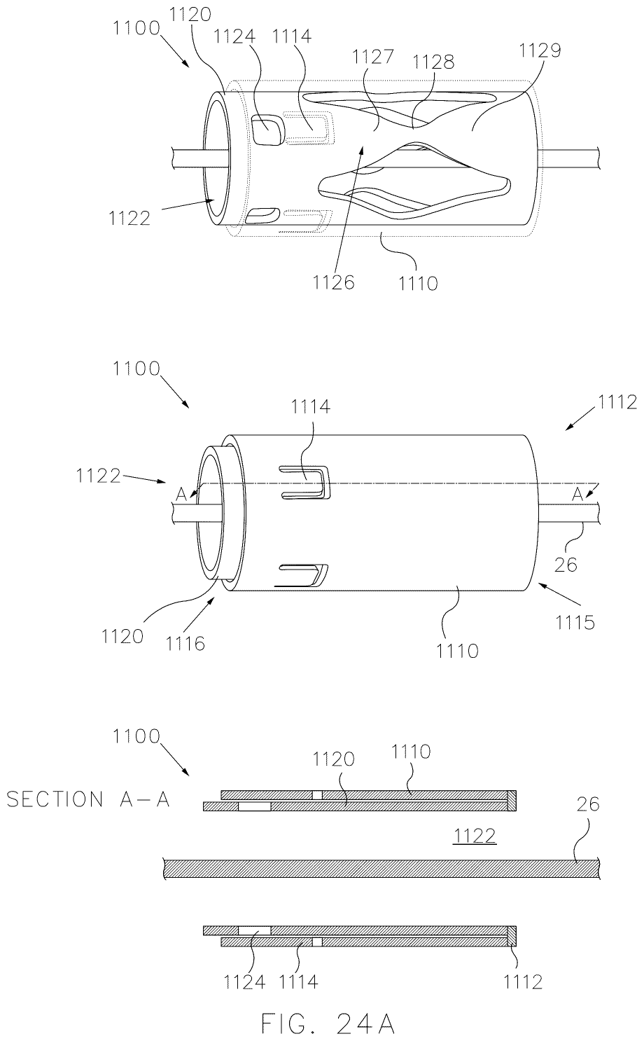

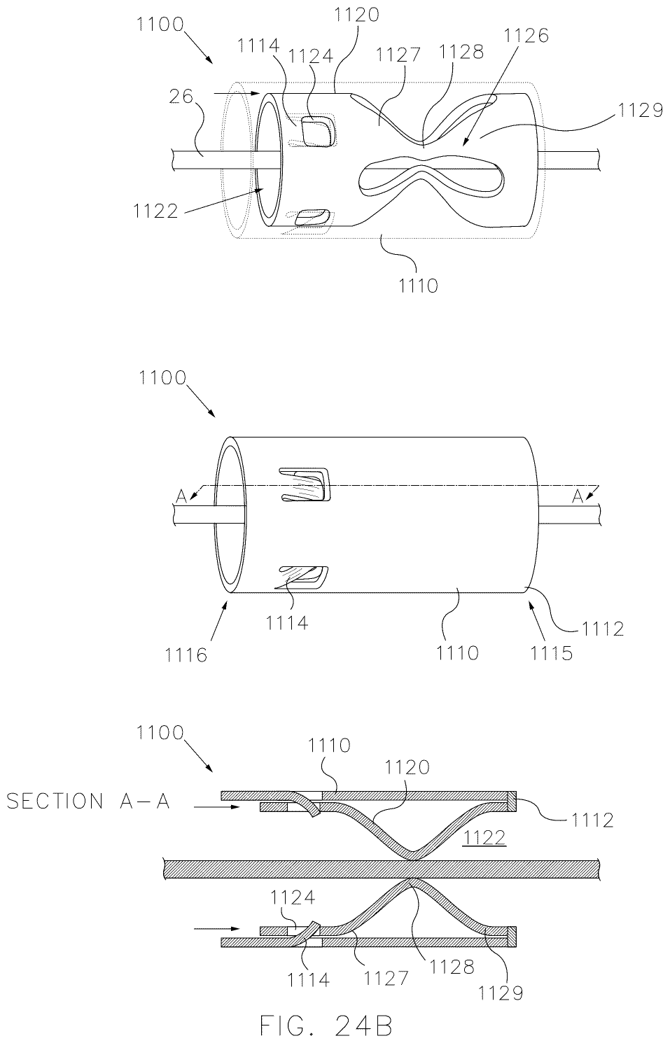

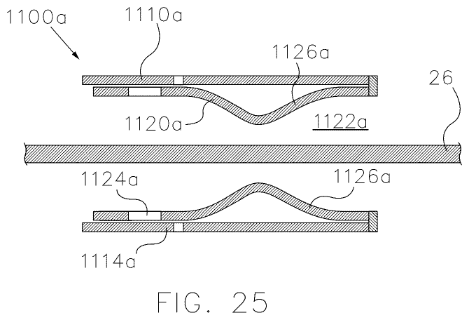

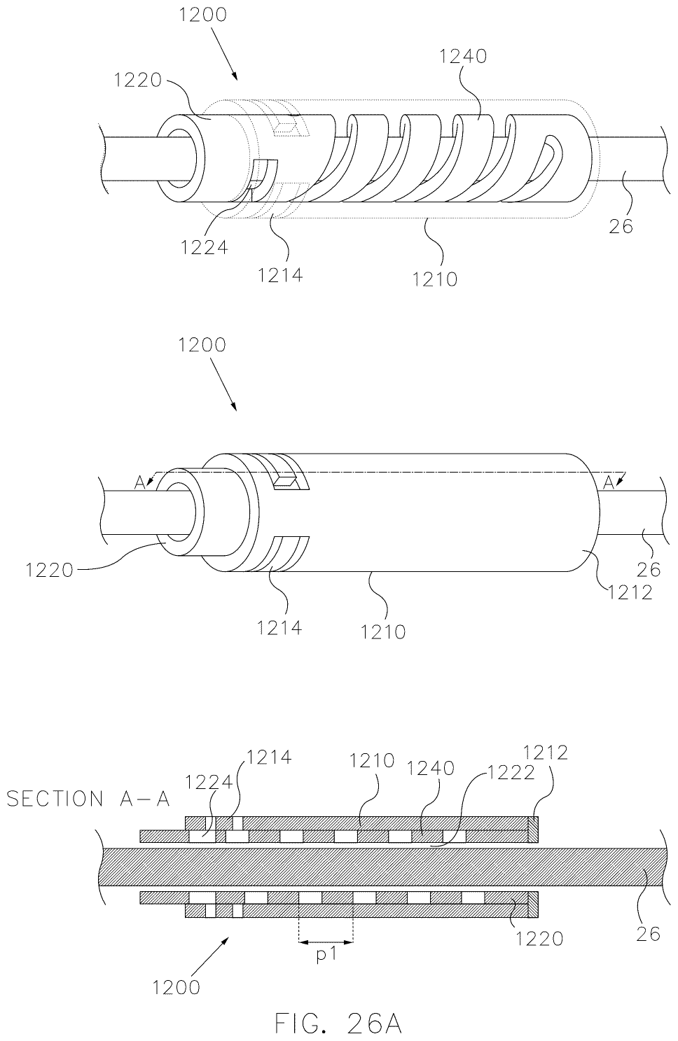

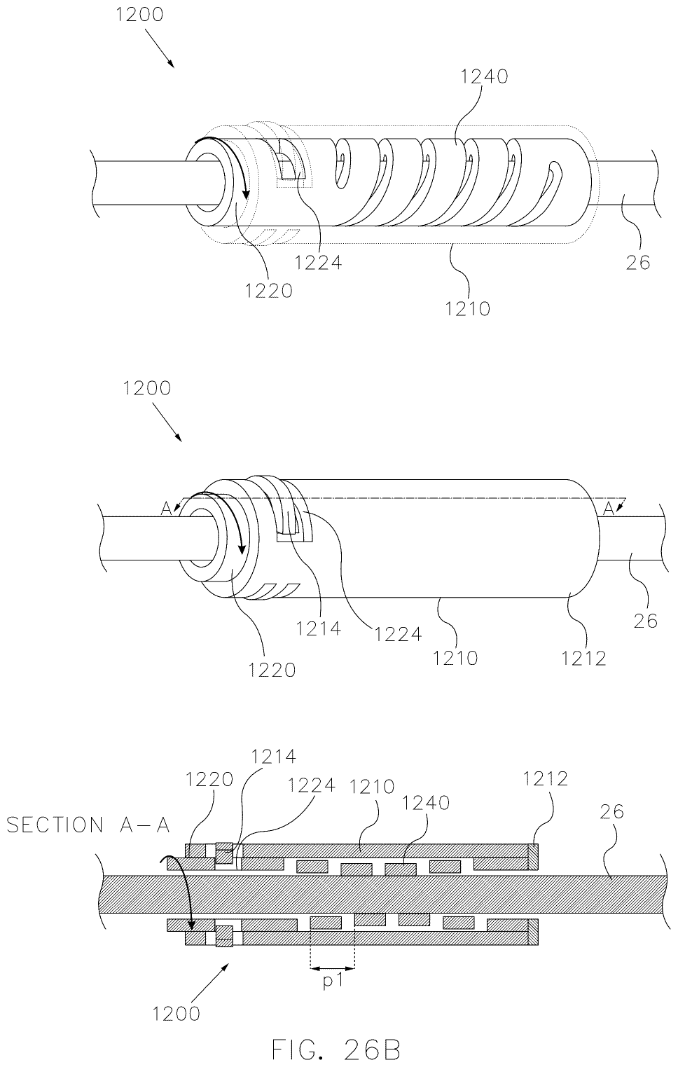

Reference is now made to FIGS. 24A-B, 25, and 26A-B, which show a variety of locks 1100, 1100a, and 1200 that are adapted to clamp onto a tether of an implant, in accordance with some implementations. Locks 1100, 1100a, and/or 1120 may be a variant of, or used instead of, any of the locks described herein, e.g., in the place of locks 24 and/or 990 mentioned hereinabove. For example, and as mentioned with reference to lock 24, locks 1100, 1100a, and/or 1120 may be locked onto a tether of an implant (e.g., tether 26 of any of the implants mentioned hereinabove), in order to maintain tension in the implant. Alternatively or additionally, locks 1100, 1100a, and/or 1120 may be used in a surgical procedure, e.g., as suture lock used in place of a knot.

Each of locks 1100, 1100a, and 1200 comprises an outer tube and an inner tube that is disposed coaxially within the outer tube.

In each of FIGS. 24A, 24B, 26A, and 26B, the central image is a perspective view of the respective lock on tether 26; the upper image shows the same view but with the outer tube of the lock transparent in order to show the inner tube of the lock; and the lower image is a longitudinal cross-section indicated by A-A. For simplicity, FIG. 25 shows just a cross-section of lock 1100a.

FIGS. 24A-B schematically illustrate lock 1100, in accordance with some implementations. Lock 1100 comprises an outer tube 1110, and an inner tube 1120 positioned coaxially within the outer tube. Tether 26 extends through lock 1100 via a lumen 1122 defined by inner tube 1120.

FIG. 24A shows lock 1100 in an unlocked state, in which tether 26 is slidable through the lock (i.e., through lumen 1122 thereof), and FIG. 24B shows the lock in a locked state in which the lock is locked to the tether.

Inner tube 1120 is fixed to outer tube 1110 at an attachment point 1112, e.g., via welding. In some implementations, and as shown, attachment point 1112 is disposed at a first end 1115 of lock 1100, e.g., a base of the inner tube is fixed to a base of the outer tube.

In some implementations, and as shown by the transition between FIGS. 24A and 24B, pushing the inner tube further into the outer tube and towards attachment point 1112 causes part of the inner tube (e.g., a mid-section of the inner tube) to bow (e.g., buckle) medially, thereby clamping onto tether 26 within lumen 1122, and thereby locking the lock to the tether. That is, inner tube 1120 is axially compressible towards attachment point 1112 in a manner that causes the inner tube to clamp to tether 26, e.g., such that a diameter of the inner tube narrows and thus presses against the tether. This may be achieved by pushing against an end of inner tube 1120, e.g., an end of the inner tube that is situated at a second end 1116 of lock 1100. For example, and as shown, in the unlocked state of lock 1100, at second end 1116, inner tube 1120 may jut out of outer tube 1110 (FIG. 24A), and transitioning the lock to its locked state is achieved by pushing the end of the inner tube towards the attachment point, thereby reducing (e.g., eliminating) the amount by which the inner tube juts out of the outer tube (FIG. 24B). In some implementations, once inner tube clamps to tether 26, tether 26 is no longer slidable within lock 1100, and the lock is thus locked to the tether (e.g., thereby maintaining tension in the tether).

In some implementations, in order to maintain lock 1100 in its locked state (e.g., to prevent axial decompression of the inner tube), the lock may define a snap-fit mechanism between outer tube 1110 and inner tube 1120, such that the inner tube snap-fits to the outer tube once the inner tube has been pushed a predetermined amount into the outer tube.

The snap-fit mechanism may be provided by inner tube 1120 defining a first snap-fit component, and outer tube 1110 defining a second snap-fit component that is complementary to the first snap-fit component. In the example shown, the first snap-fit component is a window 1124, and the second snap-fit component is a tongue 1114. When inner tube 1120 is axially compressed by the predetermined amount tongue 1114 becomes aligned (e.g., axially aligned) with window 1124 and, responsively this alignment, automatically protrudes (e.g., snaps) into the window (FIG. 24B). Due to the geometry and/or angular disposition of tongue 1114 and window 1124, axial decompression of the inner tube is inhibited.

In some implementations, and as shown, the bowing (e.g., buckling) of inner tube 1120 may result in the inner tube acquiring an overall hourglass shape having a waist that clamps to the tether (FIG. 24B). In some implementations, inner tube 1120 defines a plurality of struts 1126 extending longitudinally, e.g., between the ends of the inner tube. Each of struts 1126 may itself be hour-glass shaped, e.g., having a first bulb 1127, a second bulb 1129, and a waist 1128 therebetween. In some implementations, the bowing (e.g., buckling) of inner tube 1120 moves bulb 1127 towards bulb 1129 and causes the struts to bow medially, such that waists 1128 collectively clamp to tether 26, thereby locking the inner tube to the tether. At each end of inner tube 1120 (e.g., axially beyond struts 1126) the inner tube may be annular, e.g., may define a complete ring.

FIG. 25 illustrates a lock 1100a, in accordance with some implementations. Lock 1100a can be considered to be a variant of lock 1100 that is as described for lock 1100 except that its struts 1126a are preset to bow medially, to some degree, even in the unlocked state of the lock. Thus, the inner tube 1120a of the lock may have an hourglass shape in both the unlocked state of the lock and in the locked state of the lock. In some implementations, axially compressing inner tube 1120a causes the waists of struts 1126a to bow further medially inwards, causing them to contact against and clamp to tether 26. This preset hourglass shape of inner tube 1120 may advantageously require less force and/or movement in order for the inner tube to clamp onto the tether, compared to lock 1100. Alternatively or additionally, this preset hourglass shape may advantageously maintain tether 26 centrally within lumen 1122a of the lock, and thereby in an appropriate position to be clamped by struts 1126a.

FIGS. 26A-B schematically illustrate lock 1200, in accordance with some implementations. Lock 1200 may be similar to lock 1100 except that, rather than pushing the inner tube towards the attachment point in order to clamp the inner tube onto the tether, the inner tube is twistable inwardly towards the attachment point such that the inner tube “wrings” the tether within the lumen, thereby clamping to the tether.

Lock 1200 comprises an outer tube 1210 fixed (e.g., welded) to an inner tube 1220 at an attachment point 1212. Inner tube 1220 has a lumen 1222, and is shaped to define a helix 1240 about the lumen. Helix 1240 has a series of turns and a pitch p1. Helix 1240 may be a single-helix or a double-helix. Lock 1200 is locked to tether 26 by applying torque to inner tube 1220 (e.g., while providing a reference force to outer tuber 1210), such that helix 1240 twists inwardly to clamp onto the tether within lumen 1222.

The torque may be applied to an interface end 1228 of inner tube 1220, e.g., the end opposite to attachment point 1212. Interface end 1228 may jut out of outer tube 1210, e.g., as shown. Interface end 1228 (e.g., an outer surface thereof) may be keyed and/or may have notches cut therein in order for a tool to apply the torque thereto. Outer tube 1210 may similarly be keyed and/or have notches cut therein in order for the tool to apply the reference force thereto.

It is to be noted that the conformational change in helix 1240 resulting from the application of torque includes a reduction of pitch p1, and thereby a shortening (i.e., axial compression) of tube 1220.

In some implementations, and similarly to as described hereinabove with reference to lock 1100, lock 1200 comprises a snap-fit mechanism that is used to maintain lock 1200 locked to tether 26 (e.g., to prevent axial decompression of inner tube 1220 and/or untwisting of helix 1240). In some implementations, rotation of inner tube 1220 relative to outer tuber 1210 causes rotation of the snap-fit component of the inner tube (e.g., of a window 1224) relative to the snap-fit component of the outer tube (e.g., a tongue 1214), the snap-fit components are brought into alignment and thus automatically snap-fit to each other. This alignment may include rotational alignment and/or axial alignment, e.g., as shown. Thus, upon a predetermined amount of axial compression (and/or twisting) of the inner tube, tongue 1214 moves into rotational and/or axial alignment with window 1224 and thus automatically protrudes into the window.

Reference is again made to FIGS. 24A-26B. In some implementations, any of locks 1100, 1100a, and/or 1200 can be transluminally advanced to the heart by sliding the lock over and along tether 26 while the lock is in its unlocked state. A rod of a delivery assembly may be used to advance the lock towards the heart by pushing the lock distally (e.g., within a catheter of the delivery assembly) over and along the tether. Once the lock is positioned at the heart and/or once the tether is tensioned appropriately (e.g., as described with reference to other figures hereinabove), the lock is locked to the tether by axially compressing the inner tube towards the attachment point (e.g., by axially pushing and/or twisting the inner tube), such that the inner tube clamps to the tether within the lumen. The locking force and/or a reference force may be provided by the delivery assembly (e.g., by the rod thereof) that was used to push the lock to the heart, e.g., by pushing and/or twisting distally against the inner tube. As described hereinabove, the inner tube may be compressed towards the attachment point until the inner tube snap-fits to the outer tube, thereby preventing axial decompression of the inner tube.

In some implementations, any of locks 1100, 1100a, and/or 1200 can have multiple stable (e.g., locked) states, e.g., allowing various degrees of clamping of tether 26. In some implementations, this is provided by multiple snap-fit mechanisms arranged in a rachet-like arrangement, e.g., by the inner tube defining multiple windows along its length, such that axially compressing the inner tube further into the outer tube once a tongue on the outer tube protrudes into a first window of the mechanism causes the tongue to protrude into a second window that is further towards the attachment point than the first window.

It may be particularly advantageous, e.g., with respect to manufacturing and/or reliability—that locks 1100, 1100a, and/or 1200 can be formed (e.g., cut) from merely two pieces of stock tubing that are fixed to each other at an attachment point (e.g., at one end). In some implementations, the stock tubing may have a wall thickness of at least 75 microns (e.g., at least 100 microns) and/or no more than 600 microns (e.g., no more than 400 microns), such as 75-600 microns (e.g., 75-400 microns, such as 100-400 microns).

In some implementations, the outer tube of the lock (e.g., the stock tubing from which it is cut) has an outer diameter of at least 1500 microns (e.g., at least 2000 microns) and/or no more than 6000 microns (e.g., no more than 4000 microns). For example, the outer diameter of the outer tube may be 1500-6000 microns (e.g., 1500-4000 microns, such as 2000-4000 microns).

In some implementations, the outer tube of the lock has an inner diameter of at least 1350 microns (e.g., at least 1800 microns) and/or no more than 4800 microns (e.g., no more than 3200 microns). For example, the inner diameter of the outer tube may be 1350-4800 microns (e.g., 1350-3200 microns, such as 1800-3200 microns).

In some implementations, the inner tube of the lock (e.g., the stock tubing from which it is cut) has an outer diameter of at least 1350 microns (e.g., at least 1800 microns) and/or no more than 4800 microns (e.g., no more than 3200 microns). For example, the outer diameter of the inner tube may be 1350-4800 microns (e.g., 1350-3200 microns, such as 1800-3200 microns).

In some implementations, the inner tube has an inner diameter of at least 1000 microns (e.g., at least 1600 microns) and/or no more than 3800 microns (e.g., no more than 2500 microns). For example, the inner diameter of the inner tube may be 1000-3800 microns (e.g., 1000-2500 microns, such as 1600-2500 microns).

In some implementations, the ratio between the length of the lock (L) and the outer diameter (OD) of the lock (i.e., the L:OD ratio) may be between 1.6:1 and 2.2:1. In some implementations, the ratio between OD and the thickness (T) of the stock tubing from which the outer tube is cut (i.e., the OD:T ratio) may be between 10:1 and 20:1.

In some implementations, the outer tube of the lock has a length of at least 1000 microns (e.g., at least 3000 microns) and/or no more than 10000 microns (e.g., no more than 7500 microns). For example, the length of the outer tube may be 1000-10000 microns (e.g., 1000-7500 microns, such as 3000-7500 microns). In some implementations, the inner tube of the lock has a length of at least 1000 microns (e.g., at least 3000 microns) and/or no more than 10000 microns (e.g., no more than 7500 microns). For example, the length of the inner tube may be 1000-10000 microns (e.g., 1000-7500 microns, such as 3000-7500 microns).

In some implementations, the outer tube of the lock (e.g., the stock tubing from which it is cut) comprises (e.g., is formed from, etc.) a metal such as stainless steel, cobalt chrome, titanium, and/or nitinol. In some implementations, the inner tube of the lock (e.g., the stock tubing from which it is cut) comprises (e.g., is formed from, etc.) a metal such as stainless steel, cobalt chrome, titanium, and/or nitinol. In some implementations, the outer tube of the lock comprises (e.g., is formed from, etc.) a polymer. In some implementations, the inner tube of the lock comprises (e.g., is formed from, etc.) a polymer. In some implementations, the material of the inner tube is the same as that of the outer tube. In some implementations, the material of the inner tube is different from that of the outer tube.

Example Implementations (some non-limiting examples of the concepts herein are recited below):

Example 1. A system and/or an apparatus for use at a heart of a subject, the system/apparatus comprising an implant that comprises:
a tether; and/or
a series of anchors, each of the anchors comprising:
   a tissue-engaging element, and/or
   a head, coupled to the tissue-engaging element, slidably coupled to the tether, and comprising a lock configured, upon locking thereof, to lock the head to the tether.

Example 2. The system/apparatus according to example 1, further comprising a stopper, lockable to the tether, the stopper defining: (i) a tubular wall circumscribing a lumen dimensioned to receive the tether therethrough; (ii) a window, cut out of the wall; and/or (iii) a tab, cut from the wall, and positioned opposite the window, and biased to deflect across the lumen and protrude into the window in a manner that locks the lock to the tether received through the lumen.

Example 3. The system/apparatus according to example 1, further comprising a stopper, lockable to the tether, the stopper comprising: (A) an outer tube; and/or (B) an inner tube, the inner tube (i) positioned coaxially within the outer tube, (ii) fixed to the outer tube at an attachment point, (iii) shaped to define a lumen along a longitudinal axis of the lock, the lumen dimensioned to receive the tether therethrough, and/or (iv) axially compressible towards the attachment point in a manner that causes the inner tube to clamp to the tether within the lumen.

Example 4. The system/apparatus according to example 1, further comprising a tensioning tool that comprises a grasper, and that is configured to adjust tension on the tether by, for any anchor of the series, advancing to the anchor, grasping the tether at the anchor, and forming a loop from the tether by pulling the tether through the lock, the lock configured, upon locking thereof, to lock the head to the loop.

Example 5. The system/apparatus according to example 4, wherein the grasper is configured to be advanced through the lock to grasp the tether.

Example 6. The system/apparatus according to example 4, wherein: (i) the tensioning tool comprises a tube, the grasper configured to extend through the tube, (ii) the grasper is configured to pull the loop into the tube, and/or (iii) the tensioning tool is configured to release the loop once the lock has locked the head to the loop.

Example 7. The system/apparatus according to example 6, wherein, for each of the anchors: (i) the tube is configured to unlock the lock by applying an unlocking force to the lock, and/or (ii) the lock is biased to lock in an absence of the unlocking force.

Example 8. The system/apparatus according to example 7, wherein: (i) for each of the anchors, the lock comprises a first locking arm and a second locking arm, each of the locking arms being hingedly coupled to the head, and/or (ii) the tube is configured to unlock the lock by pushing against the locking arms in a manner that causes each of the locking arms to pivot away from each other.

Example 9. The system/apparatus according to example 7, wherein the lock is configured to lock onto the loop upon withdrawal of the tube from the head such that the locking arms pivot towards each other.

Example 10. The system/apparatus according to example 1, wherein the implant further comprises a leading anchor, fixed to a distal end of the tether.

Example 11. The system/apparatus according to any one of examples 1-10, wherein, for each of the anchors, the tissue-engaging element is helical, defines a central longitudinal axis of the anchor, and is configured to be screwed into tissue of the heart by rotation of the tissue-engaging element around the central longitudinal axis.

Example 12. The system/apparatus according to example 11, wherein, for each of the anchors, the head is configured to facilitate screwing of the tissue-engaging element into the tissue while remaining slidably coupled to the tether.

Example 13. The system/apparatus according to example 11, wherein, for each of the anchors: (i) the head comprises a circumferential wall that circumscribes the central longitudinal axis, and/or (ii) the head is slidably coupled to the tether by the tether extending transversely through the head via apertures defined in the circumferential wall.

Example 14. The system/apparatus according to example 13, wherein, for each of the anchors, the tissue-engaging element is rotatable independently of the circumferential wall.

Example 15. The system/apparatus according to example 13, wherein, for each of the anchors, the tissue-engaging element is rotatably locked with respect to the circumferential wall.

Example 16. The system/apparatus according to example 13, wherein, for each of the anchors, the lock is disposed medially from the circumferential wall.

Example 17. The system/apparatus according to any one of examples 1-16, wherein, for each of the anchors, the lock defines a press surface, and is configured to lock the head to the tether by pressing the press surface against the tether.

Example 18. The system/apparatus according to example 17, wherein, for each of the anchors, the lock comprises a press plate that defines the press surface.

Example 19. The system/apparatus according to example 17, wherein, for each of the anchors, the lock comprises a spring configured to lock the head to the tether by pressing the press surface against the tether.

Example 20. The system/apparatus according to example 19, wherein, for each of the anchors, the spring is configured to press the press surface against the tether by moving the press surface axially with respect to the tissue-engaging element.

Example 21. The system/apparatus according to example 20, wherein, for each of the anchors: (A) the head comprises a casing and a push-button, and/or (B) the lock is configured such that: (i) the spring moving the press surface axially with respect to the tissue-engaging element projects the push-button laterally from the casing, and/or (ii) pressing the push-button medially unlocks the lock by moving the press surface axially with respect to the casing.

Example 22. The system/apparatus according to example 21, wherein, for each of the anchors, the press surface is defined by a piston disposed within the casing, the piston further defines a first bearing surface, and the push-button defines a second bearing surface, oblique with respect to the first bearing surface, such that pressing the push-button medially moves the press surface axially with respect to the casing by sliding the second bearing surface over the first bearing surface.

Example 23. The system/apparatus according to example 22, wherein, for each of the anchors, the first bearing surface is oblique with respect to a central longitudinal axis of the anchor.

Example 24. The system/apparatus according to example 22, wherein, for each of the anchors, the second bearing surface is oblique with respect to a central longitudinal axis of the anchor.

Example 25. The system/apparatus according to example 22, wherein, for each of the anchors, the push-button is attached to a wedge that defines the second bearing surface.

Example 26. The system/apparatus according to example 20, wherein, for each of the anchors, the spring is configured to press the press surface against the tether by moving the press surface axially away from the tissue-engaging element.

Example 27. The system/apparatus according to example 20, wherein, for each of the anchors: (i) the head defines a circumferential wall that circumscribes a central longitudinal axis of the anchor, (ii) the head is slidably coupled to the tether by the tether extending transversely through the head via an aperture defined in the circumferential wall, (iii) at least the press surface of the lock is disposed medially from the circumferential wall, and/or (iv) the spring is configured to press the press surface against the tether by moving the press surface across the aperture.

Example 28. The system/apparatus according to example 27, wherein, for each of the anchors, the tissue-engaging element is rotatable independently of the circumferential wall.

Example 29. The system/apparatus according to example 27, wherein, for each of the anchors, the tissue-engaging element is rotatably locked with respect to the circumferential wall.

Example 30. The system/apparatus according to example 27, wherein, for each of the anchors: (A) the head comprises a casing comprising a proximal part and a base part, axially separated at a mid-section at which the circumferential wall is disposed, (B) the head is coupled to the tissue-engaging element via the base part, and/or (C) the proximal part, the base part, and the tissue-engaging element are: (i) rotationally and axially locked with respect to each other, and/or (ii) rotatably coupled to the circumferential wall.

Example 31. The system/apparatus according to example 30, wherein the lock and the circumferential wall axially traverse the mid-section.

Example 32. The system/apparatus according to any one of examples 1-31, further comprising a delivery tool, configured to transluminally advance the anchors to the heart, and comprising:
a flexible tube, transluminally advanceable to the heart, and/or
a driver, dimensioned to extend through the flexible tube,
   wherein the delivery tool is configured to, for each of the anchors, sequentially:
anchor the anchor to tissue of the heart, such that a section of the tether extends to the anchor from a preceding one of the anchors,
subsequently adjust tension on the section of the tether, and/or
subsequently lock the lock.

Example 33. The system/apparatus according to example 32, wherein, for each of the anchors: (i) the lock is biased to lock, (ii) the head comprises a casing, and a push-button operatively coupled to the lock such that while the lock is unlocked the push-button projects laterally from the casing, and/or (iii) the delivery tool is configured to maintain the lock unlocked by constraining the push-button medially.

Example 34. The system/apparatus according to example 33, wherein, for each of the anchors, the delivery tool is configured: (i) to maintain the lock unlocked by the flexible tube constraining the push-button medially, and/or (ii) to lock the lock by deploying the head out of the flexible tube.

Example 35. The system/apparatus according to example 33, wherein the casing of each anchor is keyed with an anchor key, and the flexible tube is complementarily keyed with a drive key such that the delivery tool can apply torque to the anchor via rotation of the flexible tube.

Example 36. The system/apparatus according to example 35, wherein, for each anchor, the push-button is disposed at the anchor key.

Example 37. The system/apparatus according to example 36, wherein, for each anchor, the anchor key includes a sunken key, the push-button is disposed at the sunken key, and the delivery tool is configured to maintain the lock unlocked by the drive key constraining the push-button medially.

Example 38. The system/apparatus according to example 32, wherein, for each of the anchors, the lock is biased to lock, the delivery tool is configured to constrain the anchor unlocked while adjusting the tension on the section of the tether.

Example 39. The system/apparatus according to example 38, wherein, for each of the anchors, the delivery tool is configured to constrain the anchor unlocked while transluminally advancing the anchor to the heart.

Example 40. The system/apparatus according to example 38, wherein, for each of the anchors, the delivery tool is configured to constrain the anchor unlocked while anchoring the anchor to the tissue.

Example 41. The system/apparatus according to example 32, wherein, for each of the anchors, the delivery tool is configured to transluminally advance the anchor to the heart subsequently to anchoring the preceding one of the anchors to the tissue.

Example 42. The system/apparatus according to example 41, wherein, for each of the anchors, the delivery tool is configured to transluminally advance the anchor to the heart subsequently to locking the lock of the preceding one of the anchors.

Example 43. A system for use at a tissue of a heart of a subject, the system comprising:
an implant that comprises:
   an elongate member,
   multiple anchors, and/or
   multiple locks; and/or
a delivery tool, configured to:
   implant the implant at the tissue, such that the elongate member defines a path along the tissue, by:
      using one or more of the anchors to anchor a first section of the elongate member along a first portion of the path, and/or
      subsequently, using one or more of the anchors to anchor a second section of the elongate member along a second portion of the path, and contract the tissue by:
      subsequently to anchoring the first section and prior to anchoring the second section, contracting the first portion of the path by reducing a length of the first section,
      while the first portion of the path remains contracted, using at least a first lock of the multiple locks to fix the length of the first section,
      subsequently to anchoring the second section, and while the length of the first section remains fixed, contracting the second portion of the path by reducing a length of the second section, and/or
      while the second portion of the path remains contracted, using at least a second lock of the multiple locks to fix the length of the second section.

Example 44. A system and/or an apparatus for use at a heart of a subject, the system/apparatus comprising:
an implant, comprising:
   a tether;
   a series of anchors, each of the anchors comprising:
      a tissue-engaging element, and/or
      a head, coupled to the tissue-engaging element, and slidably coupled to the tether;
multiple tension-modifiers; and/or
a delivery tool, comprising:
   a tube, transluminally advanceable to the heart,
   an anchor driver, configured to advance the anchors through the tube and, for each of the anchors sequentially, intracardially anchor the anchor to tissue of the heart by driving the tissue-engaging element into the tissue, and/or
   an applicator, configured to intracardially connect the multiple tension-modifiers to the implant.

Example 45. The system/apparatus according to example 44, wherein the applicator is configured to intracardially connect the tension-modifiers to adjacent anchors of the series.

Example 46. The system/apparatus according to any one of examples 44-45, wherein the applicator is configured to intracardially connect the tension-modifiers to the tether.

Example 47. The system/apparatus according to example 46, wherein the applicator is configured to intracardially connect the tension-modifiers to the tether between adjacent anchors of the series.

Example 48. The system/apparatus according to any one of examples 44-47, wherein each of the tension-modifiers is a connector, configured to connect a respective pair of adjacent anchors of the series.

Example 49. The system/apparatus according to example 48, wherein each of the tension-modifiers is configured to draw the respective pair of anchors toward each other.

Example 50. The system/apparatus according to example 48, wherein each of the tension-modifiers is configured to inhibit the respective pair of anchors from moving away from each other.

Example 51. The system/apparatus according to any one of examples 44-50, wherein each of the tension-modifiers is a spacer, configured to be connected to the tether between a respective pair of anchors of the series.

Example 52. The system/apparatus according to example 51, wherein each of the spacers is tubular.

Example 53. The system/apparatus according to example 51, wherein, for each of the spacers, the applicator is configured to intracardially connect the spacer to the tether while the spacer is constrained in an open state in which the spacer has a substantially C-shaped cross section.

Example 54. The system/apparatus according to example 51, wherein the tube defines a primary lumen, the anchor driver is configured to advance each of the anchors out of the primary lumen, and the applicator is configured to advance each of the spacers out of a secondary lumen disposed laterally from the primary lumen.

Example 55. A method for use at a tissue of a heart of a subject, the method comprising:
percutaneously implanting an elongate implant along the tissue such that the implant defines a path along the tissue by:
   anchoring a first section of the implant along a first portion of the path, and/or
   subsequently anchoring a second section of the implant along a second portion of the path;
subsequently to anchoring the first section and prior to anchoring the second section:
   contracting the first portion of the path by reducing a length of the first section, and/or
   while the first portion of the path remains contracted, fixing the length of the first section; and/or
subsequently to anchoring the second section, and while the length of the first section remains fixed:
   contracting the second portion of the path by reducing a length of the second section, and/or
   while the second portion of the path remains contracted, fixing the length of the second section.

Example 56. The method according to example 55, wherein: (i) the implant includes a first segment, a second segment, and/or a third segment, telescopically coupled to each other in series, (ii) the first section of the implant includes part of the first segment and part of the second segment, (iii) the second section of the implant includes part of the second segment and part of the third segment, (iv) contracting the first portion of the path comprises contracting the first portion of the path by reducing the length of the first section by telescopically contracting the first segment into the second segment, and/or (v) contracting the second portion of the path comprises contracting the second portion of the path by reducing the length of the second section by telescopically contracting the second segment into the third segment.

Example 57. The method according to example 56, wherein: (i) fixing the length of the first section comprises locking the first segment to the second segment, and/or (ii) fixing the length of the second section comprises locking the second segment to the third segment.

Example 58. The method according to example 56, wherein: (i) the first segment includes a first anchor, the second segment includes a second anchor, and/or the third segment includes a third anchor, (ii) anchoring the first section of the implant comprises anchoring the first anchor and the second anchor to the tissue, and/or (iii) anchoring the second section of the implant comprises anchoring the second anchor and the third anchor to the tissue.

Example 59. The method according to any one of examples 55-58, wherein: (i) the implant includes a tether, (ii) contracting the first portion of the path comprises contracting the first portion of the path by reducing a length of the first section by applying tension to the tether, and/or (iii) contracting the second portion of the path comprises contracting the second portion of the path by reducing a length of the second section by applying tension to the tether.

Example 60. The method according to example 59, wherein percutaneously implanting the implant along the tissue comprises implanting the implant along the tissue such that the tether defines the path along the tissue.

Example 61. The method according to example 59, wherein: (i) fixing the length of the first section comprises locking a first lock to the tether, and/or (ii) fixing the length of the second section comprises locking a second lock to the tether.

Example 62. The method according to example 61, further comprising percutaneously advancing the first lock to the heart subsequently to anchoring the first section of the implant along the first portion of the path.

Example 63. The method according to example 62, wherein advancing the first lock to the heart comprises advancing the first lock to the heart prior to anchoring the second section of the implant along the second portion of the path.

Example 64. The method according to example 62, wherein percutaneously advancing the first lock to the heart comprises percutaneously sliding the lock over and along the tether.

Example 65. The method according to example 61, wherein:
the implant includes a first locking anchor and a second locking anchor, each including:
   a tissue-engaging element, and/or
   a head that is attached to the tissue-engaging element, is slidably coupled to the tether, and/or includes a lock,
percutaneously implanting the implant along the tissue comprises anchoring the first and second locking anchors to the tissue,
the first lock is the lock of the first locking anchor, fixing the length of the first section comprising locking the lock of the first locking anchor to the tether, and/or
the second lock is the lock of the second locking anchor, fixing the length of the second section comprising locking the lock of the second locking anchor to the tether.

Example 66. The method according to example 65, further comprising percutaneously sliding each of the first and second locking anchors over and along the tether to the heart.

Example 67. The method according to example 66, wherein: (i) percutaneously implanting the implant along the tissue comprises securing a distal part of the tether to the tissue by anchoring to the tissue a leading anchor, coupled to the tether, and/or (ii) percutaneously sliding each of the first and second locking anchors over and along the tether to the heart comprises, subsequently to securing the distal part of the tether to the tissue by anchoring the leading anchor to the tissue, percutaneously sliding each of the first and second locking anchors distally over and along the tether toward the leading anchor.

Example 68. The method according to example 65, wherein anchoring the first and second locking anchors to the tissue comprises anchoring the first and second locking anchors to the tissue using an anchor driver.

Example 69. The method according to example 68, wherein, for each of the first and second locking anchors, the lock is biased toward locking, and anchoring the locking anchor to the tissue comprises anchoring the locking anchor to the tissue while the anchor driver constrains the lock unlocked.

Example 70. The method according to example 69, wherein, for each of the first and second locking anchors, locking the lock to the tether comprises releasing the lock such that the lock responsively locks.

Example 71. The method according to example 69, wherein, for each of the first and second locking anchors, anchoring the locking anchor to the tissue while the anchor driver constrains the lock unlocked comprises anchoring the locking anchor to the tissue while the anchor driver constrains the lock unlocked by inhibiting a push-button of the head from moving laterally outward from a casing of the head.

Example 72. The method according to example 68, wherein, for each of the first and second locking anchors, locking the lock to the tether comprises, using the anchor driver, applying a locking force to the lock.

Example 73. The method according to example 72, wherein, for each of the first and second locking anchors, the locking force is a rotational locking force, and/or applying the locking force to the lock comprises applying the rotational locking force to the lock.

Example 74. A method for use at a tissue of a heart of a subject, the method comprising:
percutaneously anchoring a first anchor of an implant to a first site of the tissue;
subsequently, percutaneously anchoring a second anchor of the implant to a second site of the tissue, a first portion of the tissue being disposed between the first site and the second site;
subsequently, contracting the first portion of the tissue by drawing together the first anchor and the second anchor;
while the first portion of the tissue remains contracted, fixing a distance between the first anchor and the second anchor;
subsequently, percutaneously changing a distance between a third anchor of the implant and the second anchor by sliding the third anchor with respect to the second anchor;
subsequently, anchoring the third anchor to a third site of the tissue, a second portion of the tissue being disposed between the second site and the third site;
subsequently, while the distance between the first anchor and the second anchor remains fixed, contracting the second portion of the tissue by drawing together the second anchor and the third anchor; and/or
while the second portion of the tissue remains contracted, fixing a distance between the second anchor and the third anchor.

Example 75. The method according to example 74, wherein:
the implant includes a tether,
percutaneously anchoring the second anchor comprises percutaneously anchoring the second anchor while the tether is coupled to the first anchor and is slidably coupled to the second anchor,
drawing together the first anchor and the second anchor comprises sliding the tether with respect to the second anchor,
fixing the distance between the first anchor and the second anchor comprises inhibiting sliding of the tether with respect to the second anchor by locking a first lock to the tether,
drawing together the second anchor and the third anchor comprises sliding the tether with respect to the third anchor, and/or
fixing the distance between the second anchor and the third anchor comprises locking a second lock to the tether.

Example 76. The method according to example 75, wherein: (i) sliding the tether with respect to the second anchor comprises sliding the tether in a first direction with respect to the second anchor, (ii) locking the first lock to the tether comprises inhibiting sliding of the tether in a second direction with respect to the second anchor by locking the first lock to the tether, the second direction being opposite to the first direction, and/or (iii) the method further comprises inhibiting further sliding of the tether in the first direction with respect to the second anchor by locking a third lock to the tether.

Example 77. The method according to example 76, wherein locking the third lock to the tether comprises locking the third lock to the tether prior to drawing together the second anchor and the third anchor.

Example 78. A method for use at a tissue of a heart of a subject, the method comprising:
percutaneously implanting a first section of an implant along a first portion of the tissue by anchoring a first anchor of the implant to a first site of the tissue, and anchoring a second anchor of the implant to a second site of the tissue, the first portion of the tissue being disposed between the first site and the second site;
subsequently, contracting the first portion of the tissue by reducing a length of the first section;
while the first portion of the tissue remains contracted, fixing the length of the first section;
subsequently, percutaneously implanting a second section of the implant along a second portion of the tissue by anchoring a third anchor of the implant to a third site of the tissue;
subsequently, while the length of the first section remains fixed, contracting the second portion of the tissue by reducing a length of the second section; and/or
while the second portion of the tissue remains contracted, fixing the length of the second section.

Example 79. The method according to example 78, wherein: (i) the second portion of the tissue is disposed between the second site and the third site, and/or (ii) implanting the second section of the implant along the second portion of the tissue comprises implanting the second section of the implant along the second portion of the tissue that is disposed between the second site and the third site.

Example 80. The method according to example 78, wherein: (i) the second portion of the tissue is disposed between the third site and a fourth site, and/or (ii) implanting the second section of the implant along the second portion of the tissue comprises implanting the second section of the implant along the second portion of the tissue by anchoring the third anchor of the implant to the third site of the tissue, and/or anchoring a fourth anchor of the implant to the fourth site of the tissue.

Example 81. A system and/or an apparatus for use at a heart of a subject, the system/apparatus comprising:
a segmented implant that comprises multiple segments, telescopically coupled to each other in series, each of the segments comprising an anchor; and/or
a delivery tool, configured to:
   percutaneously deliver the implant to the heart,
   anchor each of the anchors to tissue of the heart, and/or
   contract the tissue by telescopically contracting the implant while each of the anchors remain anchored to the tissue.

Example 82. The system/apparatus according to example 81, wherein the delivery tool is configured to telescopically extend the implant within the heart.

Example 83. The system/apparatus according to any one of examples 81-82, wherein each of the segments is articulatable with respect to an adjacent one of the segments.

Example 84. The system/apparatus according to any one of examples 81-83, wherein each of the segments further comprises a lock, configured such that, for each of the segments, locking of the lock fixes an amount of telescoping between the segment and an adjacent one of the segments.

Example 85. The system/apparatus according to any one of examples 81-84, wherein: (A) each of the anchors is biased toward assuming an anchoring position, and/or (B) the delivery tool is configured: (i) to percutaneously deliver the implant to the heart while each of the anchors is constrained in a delivery position, and/or (ii) within the heart, release each of the anchors to allow each of the anchors to responsively move toward the anchoring position.

Example 86. The system/apparatus according to example 85, wherein the implant has a delivery state in which, for each of the segments, the anchor is constrained in the delivery position by an adjacent one of the segments.

Example 87. The system/apparatus according to example 86, wherein, in the delivery state, for each of the segments, the anchor is constrained in the delivery position by being disposed within an adjacent one of the segments.

Example 88. The system/apparatus according to example 87, wherein, for each of the segments, the delivery tool is configured to release the anchor by telescopically extending the segment out of the adjacent one of the segments.

Example 89. The system/apparatus according to any one of examples 81-88, wherein the delivery tool is configured to: (A) secure a first section of the implant along a first portion of a path along the tissue by: (i) anchoring the anchor of a first segment of the multiple segments to a first site on the tissue, and/or (ii) anchoring the anchor of a second segment of the multiple segments to a second site on the tissue, (B) secure a second section of the implant along a second portion of a path along the tissue, by anchoring the anchor of a third segment of the multiple segments to a third site on the tissue, the first section including part of the first segment and part of the second segment, and the second section including part of the second segment and part of the third segment, (C) contract the tissue at the first portion of the path by telescopically contracting the first segment into the second segment, and/or (D) contract the tissue at the second portion of the path by contracting the second segment into the third segment independently of contracting the first segment into the second segment.

Example 90. The system/apparatus according to example 89, wherein the delivery tool is configured to lock the first segment to the second segment subsequently to telescopically contracting the first segment into the second segment, and to lock the second segment to the third segment subsequently to telescopically contracting the second segment into the third segment.

Example 91. The system/apparatus according to example 90, wherein the delivery tool is configured to contract the tissue at the second portion of the path by contracting the second segment into the third segment subsequently to locking the first segment to the second segment.

Example 92. The system/apparatus according to example 91, wherein the delivery tool is configured to telescopically extend the implant within the heart by: (i) telescopically extending the first segment from the second segment prior to anchoring the anchor of the second segment, and/or (ii) telescopically extending the second segment from the third segment subsequently to anchoring the anchor of the second segment and prior to anchoring the anchor of the third segment.

Example 93. A method, comprising: (i) implanting an implant along a tissue of a heart of a subject by anchoring to the tissue a series of anchors of the implant, the anchors being slidably coupled to a tether of the implant; (ii) subsequently, applying tension to the tether; and/or (iii) subsequently, adjusting the tension by applying a tension-modifier between a pair of adjacent anchors of the series.

Example 94. The method according to example 93, wherein the tension-modifier includes a spacer, and wherein applying the tension-modifier between the pair of adjacent anchors comprises urging the pair of adjacent anchors away from each other by connecting the spacer to the tether between the pair of adjacent anchors.

Example 95. The method according to example 93, wherein the tension-modifier includes a connector, and wherein applying the tension-modifier between the pair of adjacent anchors comprises drawing the pair of adjacent anchors toward each other by connecting the connector to both anchors of the pair of adjacent anchors.

Example 96. A method, comprising: (i) identifying a subject in whom an implant has been implanted along a tissue of a heart of the subject, the implant including a series of anchors slidably coupled to a tether, and the implant having been implanted by anchoring the series of anchors to the tissue; and/or (ii) in response to the identifying, adjusting tension on the tether by applying a tension-modifier between a pair of adjacent anchors of the series.

Example 97. The method according to example 96, wherein the tension-modifier includes a spacer, and wherein applying the tension-modifier between the pair of adjacent anchors comprises urging the pair of adjacent anchors away from each other by connecting the spacer to the tether between the pair of adjacent anchors.

Example 98. The method according to example 96, wherein the tension-modifier includes a connector, and wherein applying the tension-modifier between the pair of adjacent anchors comprises drawing the pair of adjacent anchors toward each other by connecting the connector to both anchors of the pair of adjacent anchors.

Example 99. A system and/or an apparatus for use with a tether, the system/apparatus comprising a lock that comprises: (i) a tubular wall circumscribing a lumen dimensioned to receive the tether therethrough; (ii) a window, cut out of the wall; and/or (iii) a tab, cut from the wall, and positioned opposite the window, and biased to deflect across the lumen and protrude into the window in a manner that locks the lock to the tether received through the lumen.

Example 100. The system/apparatus according to example 99, wherein: (i) the window is a first window of a plurality of windows cut out of the wall, (ii) the tab is a first tab of a plurality of tabs cut from the wall, and/or (iii) each tab of the plurality of tabs is biased to deflect across the lumen and protrude into a respective window that faces that tab, in a manner that locks the lock to the tether.

Example 101. The system/apparatus according to any one of examples 99-100, further comprising a cutter, the cutter comprising: (i) a cutting element dimensioned to receive the tether therethrough, a distal part of the cutting element defining a pair of cutting edges facing each other; and/or (ii) an overtube, slidable over and along the cutting element in a manner in which the cutting edges move towards each other, thereby cutting the tether.

Example 102. The system/apparatus according to example 101, wherein the cutting element is manufactured from a single piece of stock tubing.

Example 103. The system/apparatus according to example 101, wherein the cutter is advanceable to a heart of a subject over and along the tether while maintaining the overtube stationary with respect to the cutting element.

Example 104. The system/apparatus according to example 101, wherein the cutter is adapted to transluminally advance the lock over and along the tether to a heart of a subject.

Example 105. The system/apparatus according to example 104, wherein the cutter is adapted to transluminally advance the lock over and along the tether to the heart by pushing a distal end of the cutter against a proximal end of the lock.

Example 106. The system/apparatus according to example 101, wherein the cutting element comprises a pair of arms, each arm having: (i) a proximal part that extends distally and away from the proximal part of the other arm of the pair, and/or (ii) a distal part that extends distally and towards the distal part of the other arm of the pair.

Example 107. The system/apparatus according to example 106, wherein each arm defines a discrete elbow between the proximal part and the distal part.

Example 108. The system/apparatus according to example 106, wherein each arm defines a continuous curve from the proximal part to the distal part.

Example 109. The system/apparatus according to example 106, wherein, for each arm of the pair, a cutting edge of the pair of cutting edges is defined by the distal part of the arm.

Example 110. The system/apparatus according to example 109, wherein the proximal parts extend outwardly such that the pair of arms collectively define a wider diameter than an inner diameter of the overtube, such that sliding the overtube distally over and along the proximal parts squeezes together the proximal parts, causing the arms to pivot inwardly towards each other.

Example 111. The system/apparatus according to example 110, wherein, for each arm of the pair, an angle at which the proximal part is disposed with respect to a central axis of the cutter is shallower than an angle at which the distal part is disposed with respect to the central axis.

Example 112. The system/apparatus according to example 106, wherein, for each arm of the pair, the proximal part is longer than the distal part.

Example 113. The system/apparatus according to any one of examples 99-112, further comprising an obstructing rod, adapted to maintain the lock in an unlocked state in which the tab is prevented from deflecting across the lumen and into the window.

Example 114. The system/apparatus according to example 113, wherein the rod is adapted to extend through the lumen, alongside the tether, and/or wherein withdrawing the rod from the lock transitions the lock towards a locked state in which the tab deflects across the lumen and into the window.

Example 115. A method for use with a heart of a subject, the method comprising: (A) using a cutter, transluminally pushing a lock distally over and along a tether toward the heart, the lock including: (i) a tubular wall circumscribing a lumen through which the tether extends, (ii) a window, cut out of the wall, and/or (iii) a tab, cut from the wall, and positioned opposite the window, (B) subsequently, withdrawing an obstructing rod from the lock such that the tab responsively deflects across the lumen and into the window, thereby locking the lock to the tether, (C) subsequently, cutting the tether by advancing an overtube over and along the cutter, such that the overtube squeezes a pair of cutting edges defined by the cutter towards each other, and/or (D) subsequently, withdrawing the cutter from the heart.

Example 116. The method according to example 115, wherein pushing the lock distally over and along the tether comprises pushing the lock over and along the tether while the rod extends through the lock.

Example 117. The method according to example 116, wherein pushing the lock distally over and along the tether comprises pushing the lock over and along the tether while the rod extends distally out of the cutter and through the lock.

Example 118. The method according to example 117, wherein withdrawing the rod from the lock comprises withdrawing the rod proximally into the cutter.

Example 119. A method, comprising: (i) implanting an implant along a tissue of a heart of a subject by anchoring to the tissue a series of anchors of the implant, the anchors being slidably coupled to a tether of the implant; (ii) subsequently, pulling the tether through a lock of a head of an anchor of the series to form a loop of the tether; and/or (iii) subsequently, locking the lock to the loop.

Example 120. The method according to example 119, wherein pulling the loop of the tether through the lock comprises grasping the tether and pulling the tether into the lock.

Example 121. The method according to any one of examples 119-120, wherein locking the lock to the loop comprises withdrawing an unlocking tool from the lock, such that the lock responsively transitions towards a locked state.

Example 122. The method according to any one of examples 119-121, further comprising unlocking the lock prior to pulling the tether through the lock.

Example 123. A system, for use with a tether, the system comprising:
an anchor, having an anchor head and a tissue-engaging element extending distally away from the head so as to define an anchor axis of the anchor, the anchor head:
   defining a channel that extends through the anchor head transverse to the anchor axis, and that is dimensioned to be threaded onto the tether so as to be slidable over and along the tether,
   comprising a lock, in communication with the channel, and/or
   defining a proximal opening that provides access to the channel and the lock;
and/or
a tool, comprising a grasper configured to advance through the opening and the lock to the channel.

Example 124. The system according to example 123, wherein the system comprises the tether, threaded through the channel, and wherein the grasper is configured to grasp the tether within the channel, and to form the tether into a loop by pulling the tether proximally through the lock.

Example 125. The system according to example 124, wherein the lock is configured to lock onto the loop, responsively to withdrawing the tool from the lock.

Example 126. The system according to example 125, wherein the tool comprises a tube, the grasper is configured to extend through the tube, the grasper is configured to pull the loop into the tube, and/or the grasper is configured to release the loop once the lock has locked onto the loop.

Example 127. The system according to example 126, wherein: (i) the tube is configured to unlock the lock by applying an unlocking force to the lock, and/or (ii) the lock is biased to lock in the absence of the unlocking force.

Example 128. The system according to example 127, wherein the tube is configured to access the lock via the opening.

Example 129. The system according to example 127, wherein: (i) the lock comprises a first locking arm and a second locking arm, each of the locking arms being hingedly coupled to the anchor head, and/or (ii) the tube is configured to unlock the lock by pushing against the locking arms in a manner that causes each of the locking arms to pivot away from each other.

Example 130. The system according to example 129, wherein the lock is configured to, responsively to withdrawal of the tube from the anchor head, lock onto the loop by the locking arms pivoting towards each other.

Example 131. A system and/or an apparatus for use with a tether, the system/apparatus comprising a lock that comprises:
an outer tube; and/or
an inner tube:
   positioned coaxially within the outer tube;
   fixed to the outer tube at an attachment point;
   shaped to define a lumen along a longitudinal axis of the lock, the lumen dimensioned to receive the tether therethrough; and/or
   axially compressible towards the attachment point in a manner that causes the inner tube to clamp to the tether within the lumen.

Example 132. The system/apparatus according to example 131, wherein the inner tube is deformable in a manner in which axially compressing the inner tube towards the attachment point causes the inner tube to bow inwardly against the tether.

Example 133. The system/apparatus according to any one of examples 131-132, wherein the inner tube has a first end and a second end, and/or wherein the inner tube is axially compressible towards the attachment point by moving the second end towards the first end.

Example 134. The system/apparatus according to example 133, wherein the inner tube, at each of the first end and the second end thereof, is annular.

Example 135. The system/apparatus according to example 133, wherein the attachment point is at the first end.

Example 136. The system/apparatus according to example 133, wherein the inner tube is axially compressible towards the attachment point by applying torque to the second end.

Example 137. The system/apparatus according to example 136, wherein the inner tube defines a helix having a series of turns, and/or application of the torque reduces a pitch of the helix.

Example 138. The system/apparatus according to example 137, wherein the helix is a double-helix.

Example 139. The system/apparatus according to example 133, wherein the inner tube is axially compressible towards the attachment point by pushing the second end towards the first end.

Example 140. The system/apparatus according to example 139, wherein the lumen is disposed along the longitudinal axis of the lock, and/or wherein the inner tube is axially compressible towards the attachment point by pushing one end of the inner tube along the longitudinal axis towards another end of the inner tube.

Example 141. The system/apparatus according to example 133, wherein: (i) the lock has an unlocked state in which the lock is slidable along the tether, and/or (ii) the lock is transitionable towards a locked state by axially compressing the inner tube.

Example 142. The system/apparatus according to example 141, wherein the lock is transitionable towards the locked state by moving the second end into the outer tube.

Example 143. The system/apparatus according to example 141, wherein: (i) in the unlocked state of the lock, the second end is disposed outside of the outer tube, and/or (ii) in the locked state of the lock, the second end is disposed inside the outer tube.

Example 144. The system/apparatus according to any one of examples 131-143, wherein the lock comprises a snap-fit mechanism between the inner tube and the outer tube, and/or wherein the inner tube is axially compressible towards the attachment point until the inner tube snap-fits to the outer tube, the snap-fit preventing axial decompression of the inner tube.

Example 145. The system/apparatus according to example 144, wherein the inner tube defines a window, and/or the outer tube defines a tongue adapted to extend into the window, and/or wherein the inner tube is axially compressible towards the attachment point until the tongue extends into the window.

Example 146. The system/apparatus according to example 144, wherein the outer tube defines a window, and/or the inner tube defines a tongue adapted to extend into the window, and/or wherein the inner tube is axially compressible towards the attachment point until the tongue extends into the window.

Example 147. The system/apparatus according to any one of examples 131-146, wherein the inner tube is shaped to define an hour-glass form having a first bulb, a second bulb, and/or a waist therebetween, and/or wherein the inner tube is axially compressible towards the attachment point in a manner that causes the waist to clamp to the tether.

Example 148. The system/apparatus according to example 147, wherein the inner tube is axially compressible towards the attachment point by pushing the first bulb towards the second bulb.

Example 149. The system/apparatus according to example 147, wherein each of the first bulb and the second bulb is annular.

Example 150. A method for use with a tether, the method comprising: (i) transluminally advancing a lock to a heart of a subject over and along the tether, the lock comprising an inner tube, positioned coaxially within an outer tube and fixed to the outer tube at an attachment point, the tether extending through a lumen defined by the inner tube; and/or (ii) subsequently, locking the lock to the tether by axially compressing the inner tube towards the attachment point such that the inner tube clamps to the tether within the lumen.

Example 151. The method according to example 150, wherein compressing the inner tube towards the attachment point such that the inner tube clamps to the tether comprises compressing the inner tube towards the attachment point such that a diameter of the inner tube narrows.

Example 152. The method according to any one of examples 150-151, wherein axially compressing the inner tube towards the attachment point comprises pushing the inner tube further into the outer tube.

Example 153. The method according to any one of examples 150-152, wherein the inner tube has a first end and a second end, and/or wherein axially compressing the inner tube towards the attachment point comprises axially compressing the inner tube by pushing the second end towards the first end.

Example 154. The method according to example 153, wherein the attachment point is disposed at the first end.

Example 155. The method according to example 153, wherein: (i) transluminally advancing the lock comprises transluminally advancing the lock while the second end is disposed outside of the outer tube, and/or (ii) pushing the second end towards the first end comprises pushing the second end into the outer tube.

Example 156. The method according to example 153, wherein the lumen is disposed along a longitudinal axis of the lock, and/or wherein axially compressing the inner tube towards the attachment point comprises axially compressing the inner tube by axially pushing one end of the inner tube along the longitudinal axis towards another end of the inner tube.

Example 157. The method according to any one of examples 150-156, wherein axially compressing the inner tube toward the attachment point comprises axially compressing the inner tube toward the attachment point until the inner tube snap-fits to the outer tube, the snap-fit preventing axial decompression of the inner tube.

Example 158. The method according to example 157, wherein the lock defines a window and a tongue, and/or wherein axially compressing the inner tube toward the attachment point until the inner tube snap-fits to the outer tube comprises axially compressing the inner tube toward the attachment point until the tongue protrudes into the window.

Example 159. The method according to example 158, wherein axially compressing the inner tube toward the attachment point until the tongue protrudes into the window comprises axially compressing the inner tube toward the attachment point until the tongue becomes aligned with the window and, responsively to becoming aligned with the window, automatically protrudes into the window.

Example 160. The method according to example 159, wherein axially compressing the inner tube toward the attachment point until the tongue becomes aligned with the window comprises axially compressing the inner tube toward the attachment point such that the tongue moves into axial alignment with the window.

Example 161. The method according to example 159, wherein axially compressing the inner tube toward the attachment point until the tongue becomes aligned with the window comprises axially compressing the inner tube toward the attachment point by twisting the inner tube towards the attachment point until the tongue moves into rotational alignment with the window.

Example 162. The method according to example 158, wherein the outer tube defines the tongue, and/or the inner tube defines the window, and/or wherein axially compressing the inner tube toward the attachment point until the tongue extends into the window comprises axially compressing the inner tube toward the attachment point until the tongue of the outer tube extends into the window of the inner tube.

Example 163. The method according to any one of examples 150-162, wherein axially compressing the inner tube towards the attachment point such that the inner tube clamps to the tether comprises axially compressing the inner tube towards the attachment point such that part of the inner tube bows medially against the tether.

Example 164. The method according to example 163, wherein axially compressing the inner tube towards the attachment point such that the part of the inner tube bows medially against the tether comprises axially compressing the inner tube towards the attachment point such that the inner tube acquires an hourglass shape having a waist that clamps to the tether.

Example 165. The method according to any one of examples 150-164, wherein the inner tube has a first end-portion and a second-end portion, and is shaped to define multiple struts extending between the first end-portion and the second-end portion, and/or wherein axially compressing the inner tube towards the attachment point such that the inner tube clamps to the tether comprises axially compressing the inner tube towards the attachment point such that the struts bow medially against the tether.

Example 166. The method according to example 165, wherein each of the struts has an hour-glass shape having a first bulb, a second bulb, and/or a waist therebetween, and/or wherein axially compressing the inner tube towards the attachment point such that the struts bow medially against the tether comprises axially compressing the inner tube towards the attachment point such that the waist of each of the struts clamps to the tether.

Example 167. The method according to example 166, wherein axially compressing the inner tube towards the attachment point such that the inner tube clamps to the tether comprises axially compressing the inner tube towards the attachment point in a manner in which, for each of the struts, the first bulb moves towards the second bulb.

Example 168. The method according to any one of examples 150-167, wherein axially compressing the inner tube towards the attachment point comprises axially compressing the inner tube towards the attachment point such that the inner tube twists inwardly against the tether.

Example 169. The method according to example 168, wherein the inner tube is shaped to define a helix that has a series of turns and a pitch, and/or wherein axially compressing the inner tube towards the attachment point such that the inner tube twists inwardly against the tether comprises reducing the pitch by twisting the inner tube towards the attachment point.

Example 170. The method according to example 169, wherein the helix is a double-helix, and/or wherein reducing the pitch comprises reducing the pitch of the double-helix.

Example 171. The system according to any one of the above examples of a system, wherein the implant is sterilized.

Example 172. The system/apparatus according to any one of the above examples of system/apparatus, wherein the implant is sterilized.

Example 173. The system/apparatus according to any one of the above examples of system/apparatus, wherein the delivery tool is sterilized.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein (including in the examples above) or in the references incorporated herein can be performed on a living subject (e.g., human, other animal, etc.) or on a simulation, such as a cadaver, cadaver heart, simulator, imaginary person, etc. When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc.

Any of the various systems, assemblies, devices, components, apparatuses, etc. in this disclosure (including in the examples listed above) can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

1. An apparatus for use at a heart of a subject, the apparatus comprising:
an implant that comprises:
a tether; and
a series of anchors, each of the anchors comprising:
a tissue-engaging element,
a head, coupled to the tissue-engaging element, slidably coupled to the tether, and comprising a lock that is biased to lock in a manner that locks the head to the tether; and
a push-button operatively coupled to the lock; and
a delivery tool, comprising:
a flexible tube, transluminally advanceable to the heart; and
a driver, configured to, for each of the anchors sequentially:
advance the anchor through the flexible tube to the heart while the lock is maintained unlocked by the flexible tube constraining the push-button,
anchor the anchor to tissue of the heart, and
release the push-button by deploying the head out of the flexible tube, the lock responsively locking.

2. The apparatus according to claim 1, wherein the implant further comprises a leading anchor, fixed to a distal end of the tether.

3. The apparatus according to claim 1, wherein, for each of the anchors, the tissue-engaging element is helical, defines a central longitudinal axis of the anchor, and is configured to be screwed into tissue of the heart by rotation of the tissue-engaging element around the central longitudinal axis.

4. The apparatus according to claim 3, wherein, for each of the anchors:
the head comprises a circumferential wall that circumscribes the central longitudinal axis, and
the head is slidably coupled to the tether by the tether extending transversely through the head via apertures defined in the circumferential wall.

5. The apparatus according to claim 4, wherein, for each of the anchors, the tissue-engaging element is rotatable independently of the circumferential wall.

6. The apparatus according to claim 4, wherein, for each of the anchors, the lock is disposed medially within the circumferential wall.

7. The apparatus according to claim 1, wherein, for each of the anchors,
the head comprises a casing comprising a proximal part and a base part, axially separated at a mid-section at which the circumferential wall is disposed,
the head is coupled to the tissue-engaging element via the base part, and
the proximal part, the base part, and the tissue-engaging element are:
rotationally and axially locked with respect to each other, and
rotatably coupled to the circumferential wall.

8. The apparatus according to claim 7, wherein the lock and the circumferential wall axially traverse the mid-section.

9. The apparatus according to any one claim 1, wherein the delivery tool is configured to, for each of the anchors, sequentially:
anchor the anchor to the tissue such that a section of the tether extends to the anchor from a preceding one of the anchors, and
adjust tension on the section of the tether subsequently to anchoring the anchor to the tissue but prior to releasing the push-button.

10. The apparatus according to claim 1, wherein, for each of the anchors:
the head comprises a casing,
the push-button is operatively coupled to the lock such that while the lock is unlocked the push-button projects laterally from the casing, and
the delivery tool is configured to maintain the lock unlocked by the flexible tube constraining the push-button medially.

11. The apparatus according to claim 10, wherein the casing of each anchor is keyed with an anchor key, and the flexible tube is complementarily keyed with a drive key such that the delivery tool can apply torque to the anchor via rotation of the flexible tube.

12. The apparatus according to claim 11, wherein, for each anchor, the push-button is disposed at the anchor key.

13. The apparatus according to claim 12, wherein, for each anchor, the anchor key includes a sunken key, the push-button is disposed at the sunken key, and the delivery tool is configured to maintain the lock unlocked by the drive key constraining the push-button medially.

14. The apparatus according to claim 9, wherein, for each of the anchors, the driver is configured to advance the anchor through the flexible tube to the heart subsequently to anchoring the preceding one of the anchors to the tissue.

15. The apparatus according to claim 14, wherein, for each of the anchors, the driver is configured to advance the anchor through the flexible tube to the heart subsequently to locking of the lock of the preceding one of the anchors.

16. The apparatus according to claim 10, wherein, for each of the anchors, the lock comprises (i) a press surface, and (ii) a spring configured to lock the head to the tether by pressing the press surface against the tether by moving the press surface axially with respect to the tissue-engaging element.

17. The apparatus according to claim 16, wherein, for each of the anchors:
the press surface is defined by a piston disposed within the casing, the piston further defines a first bearing surface that is oblique with respect to a central longitudinal axis of the anchor, and
pressing the push-button medially moves the press surface axially with respect to the casing by sliding the first bearing surface over a second bearing surface defined by the piston.

18. The apparatus according to claim 1, further comprising a stopper, lockable to the tether, the stopper defining:
a tubular wall circumscribing a lumen dimensioned to receive the tether therethrough;
a window, cut out of the tubular wall; and
a tab, cut from the tubular wall, and positioned opposite the window, and biased to deflect across the lumen and protrude into the window in a manner that locks the lock to the tether received through the lumen.

19. The apparatus according to claim 1, further comprising a stopper, lockable to the tether, the stopper comprising:
an outer tube; and
an inner tube:
positioned coaxially within the outer tube,
fixed to the outer tube at an attachment point,
shaped to define a lumen along a longitudinal axis of the lock, the lumen dimensioned to receive the tether therethrough, and
axially compressible towards the attachment point in a manner that causes the inner tube to clamp to the tether within the lumen.
